(19) United States
(12) Patent Application Publication
(10) Pub. No.: US 2012/0039935 A1
(43) Pub. Date: Feb. 16, 2012

(54) METHODS OF TREATING MEASLES INFECTIOUS DISEASE IN MAMMALS

(75) Inventors: Adrian Vilalta, San Diego, CA (US); Gretchen Jimenez, San Diego, CA (US); Diane E. Griffin, Baltimore, MD (US)

(73) Assignee: Vical Incorporated, San Diego, CA (US)

(21) Appl. No.: 12/817,125

(22) Filed: Jun. 16, 2010

Related U.S. Application Data
(63) Continuation of application No. 12/129,606, filed on May 29, 2008, now abandoned.
(60) Provisional application No. 60/940,673, filed on May 29, 2007.

(51) Int. Cl. A61K 39/165; A61P 31/14

(52) U.S. Cl. 424/212.1

(57) ABSTRACT

The invention provides for a measles vaccine utilizing a human codon-optimized polynucleotide encoding a measles virus polypeptide, such as HA or F protein. Optionally, the vaccine is administered with an adjuvant and is especially useful for immunizing an infant mammal.

### CROSS REFERENCE TO OTHER APPLICATIONS

This application is a continuation of U.S. patent application Ser. No. 12/129,606, entitled “METHODS OF TREATING MEASLES INFECTION DISEASE IN MAMMALS”, filed May 29, 2008, which application claims priority benefit under 35 U.S.C. §119(e) to U.S. Provisional Patent Application No. 60/940,673, titled: “METHOD OF TREATING MEASLES INFECTIOUS DISEASE IN MAMMALS”, filed May 29, 2007, the disclosures of which are hereby incorporated by reference in their entirety for all purposes.

### BACKGROUND OF THE INVENTION

Measles remains a major cause of infant mortality despite the availability of a safe and effective live attenuated virus vaccine. Recent efforts to reduce mortality through improved routine vaccination combined with mass vaccination campaigns have moved measles control toward the World Health Assembly goal of 90% reduction in mortality by 2010 (Center for Disease Control. Progress in global measles control and mortality reduction, 2000-2006. MMWR 56, 1237-1242 (2007)). One impediment to measles control remains the inability to immunize young infants due to immaturity of the immune system and interference of maternal antibody that impair immune responses to the current vaccine (Albrecht, P., et al., J. Pediatr., 91:715-178 (1977); and Gans, H. A., et al., JAMA, 280:527-532 (1998)).

Because waning of maternal antibody varies from one infant to another, many children in areas with high measles virus (MV) transmission are at risk of acquiring measles prior to vaccination (Babaniyi, O. A., et al., J. Trop. Pediatr., 41:115-117 (1995); and Black, F. L., Prog. Med. Virol., 36:1-33 (1989)). This is particularly true of children born to HIV-positive mothers who have a lower level of maternal antibodies at birth (Scott, S., et al., Clin Infect Dis., 45:1417-1424 (2007); and Moss, W. J., et al., Clin Infect Dis., 35:189-196 (2002)). Independent of maternal antibody, immaturity affects the quality and quantity of antibody produced in response to the current live attenuated vaccine with lower levels of neutralizing antibody and deficient avidity and isotype maturation compared to older infants (Gans, H. A., et al., JAMA, 280:527-532 (1998); Siegrist, C. A., Vaccine, 19:3331-3346 (2001); and Nair, N., et al., J Infect Dis., 196:1339-1345 (2007)). As a result, the recommended age for vaccination is generally 9 months in developing countries and 12 months in developed countries to balance the risk of infection with the likelihood of response to the vaccine (Halsey, N. A., et al., N. Engl. J. Med., 313:544-549 (1985)).

A vaccine that could be given under the age of 6 months would improve measles control by allowing delivery with other infant vaccines and by closing the window of susceptibility prior to delivery of the current vaccine. Increasing the dose of vaccine improved the antibody responses in young infants, but resulted in an unexpected increase in mortality for girls, so is not a viable approach to lowering the age of vaccination (Garenne, M., et al., Lancet, 338:903-907 (1991); and Holt, E. A., et al., J. Infect. Dis., 168:1087-1096 (1993)). Therefore, other strategies are necessary for development of a vaccine for young infants.

MV encodes six structural proteins of which two, hemagglutinin (HA) and fusion (F), are surface glycoproteins involved in attachment and entry. Antibodies that inhibit MV infection in neutralization assays are directed primarily against the HA protein, which also contains important CD8+ T cell epitopes (Ota, M. O., et al., J. Infect. Dis., 195:1799-1807 (2007)), with some contribution from F. See, (Polack, F., et al., Nat Med., 6:776-781 (2000)). Because protection from measles correlates best with the quality and quantity of neutralizing antibodies at the time of exposure (Polack, F., et al., Nat Med., 6:776-781 (2000); and Chen, R. T. et al., J. Infect. Dis., 162:1036-1042 (1990)) most experimental vaccines have used HA alone or HA and F for induction of MV protective immunity (Polack, F., et al., Nat Med., 6:776-781 (2000); Van Binnendijk, R. S., et al., J. Infect. Dis., 175:524-532 (1997); Pan, C. H., et al., Proc Natl. Acad Sci U.S.A., 102:11581-11588 (2005); and Zhu, Y., et al., Virology, 276:202-213 (2000)).

Several small animal models are available for testing measles vaccines, but only nonhuman primates develop disease after infection with wild type strains of MV so that protective immunity and vaccine safety can be assessed. Experimental vaccines that have been tested in nonhuman primates include immunostimulatory complexes (de Vries, P., et al., J. Gen. Virol., 69:549-559 (1988); and Stittelaar, K. J., et al., Vaccine, 21:155-157 (2002)), recombinant viral vectors (Van Binnendijk, R. S., et al., J. Infect. Dis., 175:524-532 (1997); Pan, C. H., et al., Proc Natl. Acad Sci U.S.A., 102:11581-11588 (2005); Zhu, Y., et al., Virology, 276:202-213 (2000); and Stittelaar, K. J., et al., J. Virol., 74:4236-4243 (2000)), recombinant bacterial vectors (Zhu, Y., et al., J. Infect. Dis., 176:1445-1453 (1997)) and DNA (Polack, F., et al., Nat Med., 6:776-781 (2000); Stittelaar, K. J., et al., Vaccine, 20:2022-2026 (2002); and Pasetti, M. F., et al., Clin. Pharmacol. Ther., 82:672-685 (2007)). DNA vaccines are attractive candidates for development because they do not elicit antivector immunity, are safe, relatively inexpensive to produce, may not require a cold-chain and induce strong cellular immune responses (Schalk, J. A., et al., Hum. Vaccin., 2:45-53 (2006)). However, DNA vaccines have often been disappointing when tested in humans and nonhuman primates because of the relatively poor induction of antibody (Donnelly, J. J., et al., J Immunol., 175:633-639 (2005)). Unformulated DNA vaccines encoding MV HA, F or HA+F induce sustained antibody responses of variable titer and provide partial protection from challenge in juvenile rhesus monkeys (Polack, F., et al., Nat Med., 6:776-781 (2000); and Premenko-Lanier, M., et al., Virology, 307:67-75 (2003)), but infant monkeys have poor responses suggesting that the vaccine needs improvement. Approaches to improving responses to DNA vaccines have included increasing the amount of DNA given, microparticle formulation, plasmid improvement, altered delivery and adding adjuvants (Denis-Mize, K. S., et al., Cell Immunol., 225:12-20 (2003); Kim, T. W., et al., J Clin Invest., 112:109-117 (2003); Leitner, W. W. et al., Nat Med., 9:33-39 (2003); and Kutzler, M. A., et al., J Clin Invest., 114:1241-1244 (2004)).

One class of adjuvants that has been explored is cationic lipids. Cationic lipids can be easily manufactured and are safe and well tolerated in humans and other animals (Nabel, G. J., et al., Proc Natl. Acad Sci U.S.A., 90:11307-11311 (1993); and Parker, S. E., et al., Hum. Gene Ther., 6:575-590 (1995)). Vaxfectin® is a recently introduced adjuvant for DNA vaccines that consists of an equimolar mixture of the cationic lipid GAP-DMORIE [(+)-N-(3-aminopropyl)-N,N-dimethyl-2,3-bis(cis-9-tetradecenyloxy)-1-propanaminium bromide)] and a neutral colipid DPyPE (1,2-diphytanoyl-sn-glydero-3-phosphoethanolamine) (Hartikka, J., et al., Vaccine, 19:1911-1923 (2001)). Vaxfectin® is dose-sparing, enhances production of antigen-specific antibody in small animals, including virus-neutralizing antibody, and can induce immunity to a variety of infections (Hartikka, J., et al., Vaccine, 19:1911-1923 (2001); Nukuzuma, C., et al., Viral Immunol., 16:183-189 (2003); Hermanson, G., et al., Proc Natl. Acad Sci U.S.A., 101:13601-13606 (2004); Sedegah, M., et al., Vaccine, 24:1921-1927 (2006); Hahn, U. K., et al., Vaccine, 24:4595-4597 (2006); Margalith, M., et al., Genet. Vaccines. Ther., 4:2 (2006); and Jimenez, G. S., et al., Hum. Vaccin., 3:157-164 (2007)).

However, efficacy of Vaxfectin®-formulated DNA vaccines has not been reported in humans and there is only a single study in nonhuman primates (Locher, C. P., et al., Vaccine, 22:2261-2272 (2004)). No studies have examined efficacy in very young animals.

### BRIEF SUMMARY OF THE INVENTION

The present invention is directed to enhancing the immune response of a vertebrate or mammal in need of protection against measles virus infection by administering in vivo, into a tissue of the vertebrate, at least one polynucleotide, wherein the polynucleotide comprises one or more nucleic acid fragments, where the one or more nucleic acid fragments are optionally fragments of codon-optimized coding regions operably encoding one or more measles virus polypeptides, or fragments, variants, or derivatives thereof. The present invention is further directed to enhancing the immune response of a vertebrate in need of protection against measles virus infection by administering, in vivo, into a tissue of the vertebrate, a polynucleotide described above plus at least one isolated measles virus polypeptide or a fragment, a variant, or derivative thereof. The isolated measles virus polypeptide can be, for example, a purified subunit, a recombinant protein, a viral vector expressing an isolated measles virus polypeptide, or can be an inactivated or attenuated measles virus, such as those present in conventional measles virus vaccines. According to either method, the polynucleotide is incorporated into the cells of the vertebrate in vivo, and an immunologically effective amount of an immunogenic epitope of the encoded measles virus polypeptide, or a fragment, variant, or derivative thereof, is produced in vivo. When utilized, an isolated measles virus polypeptide or a fragment, variant, or derivative thereof is also administered in an immunologically effective amount.

According to the present invention, the polynucleotide can be administered either prior to, at the same time (simultaneously), or subsequent to the administration of the isolated measles virus polypeptide. The measles virus polypeptide or fragment, variant, or derivative thereof encoded by the polynucleotide comprises at least one immunogenic epitope capable of eliciting an immune response to measles virus in a vertebrate. In addition, an isolated measles virus polypeptide or fragment, variant, or derivative thereof, when used, comprises at least one immunogenic epitope capable of eliciting an immune response in a vertebrate. The measles virus polypeptide or fragment, variant, or derivative thereof encoded by the polynucleotide can, but need not, be the same protein or fragment, variant, or derivative thereof as the isolated measles virus polypeptide which can be administered according to the method.

The polynucleotide of the invention can comprise a nucleic acid fragment, where the nucleic acid fragment is a fragment of a codon-optimized coding region operably encoding any measles virus polypeptide or fragment, variant, or derivative thereof, including, but not limited to, HA, or F proteins or fragments, variants or derivatives thereof. A polynucleotide of the invention can also encode a derivative fusion protein, wherein two or more nucleic acid fragments, at least one of which encodes a measles virus polypeptide or fragment, variant, or derivative thereof, are joined in frame to encode a single polypeptide, such as, but not limited to, HA or F. Additionally, a polynucleotide of the invention can further comprise a heterologous nucleic acid or nucleic acid fragment. Such heterologous nucleic acid or nucleic acid fragment may encode a heterologous polypeptide fused in frame with the polynucleotide encoding the measles virus polypeptide, e.g., a hepatitis B core protein or a secretory signal peptide. Preferably, the polynucleotide encodes a measles virus polypeptide or fragment, variant, or derivative thereof comprising at least one immunogenic epitope of measles virus, wherein the epitope elicits a B-cell (antibody) response, a T-cell (e.g., CTL) response, or both.

Similarly, the isolated measles virus polypeptide or fragment, variant, or derivative thereof to be delivered (either a recombinant protein, a purified subunit, or viral vector expressing an isolated measles virus polypeptide, or in the form of an inactivated measles virus vaccine) can be any isolated measles virus polypeptide or fragment, variant, or derivative thereof, including but not limited to the HA, or F proteins or fragments, variants or derivatives thereof. In certain embodiments, a derivative protein can be a fusion protein. In other embodiments, the isolated measles virus polypeptide or fragment, variant, or derivative thereof can be fused to a heterologous protein, e.g., a secretory signal peptide or the hepatitis B virus core protein. Preferably, the isolated measles virus polypeptide or fragment, variant, or derivative thereof comprises at least one immunogenic epitope of measles virus, wherein the antigen elicits a B-cell antibody response, a T-cell antibody response, or both.

Nucleic acids and fragments thereof of the present invention can be altered from their native state in one or more of the following ways. First, a nucleic acid or fragment thereof which encodes a measles virus polypeptide or fragment, variant, or derivative thereof can be part or all of a codon-optimized coding region, optimized according to codon usage in the animal in which the vaccine is to be delivered. In addition, a nucleic acid or fragment thereof which encodes a measles virus polypeptide can be a fragment which encodes only a portion of a full-length polypeptide, and/or can be mutated so as to, for example, remove from the encoded polypeptide non-desired protein motifs present in the encoded polypeptide or virulence factors associated with the encoded polypeptide. For example, the nucleic acid sequence could be mutated so as not to encode a membrane anchoring region that would prevent release of the polypeptide from the cell. Upon delivery, the polynucleotide of the invention is incorporated into the cells of the vertebrate in vivo, and a prophylactically or therapeutically effective amount of an immunologic epitope of a measles virus is produced in vivo.

The invention further provides immunogenic compositions comprising at least one polynucleotide, wherein the polynucleotide comprises one or more nucleic acid fragments, where each nucleic acid fragment is a fragment of a codon-optimized coding region encoding a measles virus polypeptide or a fragment, a variant, or a derivative thereof and immunogenic compositions comprising a polynucleotide as described above and at least one isolated measles virus polypeptide or a fragment, a variant, or derivative thereof. Such compositions can further comprise, for example, carriers, excipients, transfection facilitating agents, and/or adjuvants as described herein.

The immunogenic compositions comprising a polynucleotide and an isolated measles virus polypeptide or fragment, variant, or derivative thereof as described above can be provided so that the polynucleotide and protein formulation are administered separately, for example, when the polynucleotide portion of the composition is administered prior (or subsequent) to the isolated measles virus polypeptide portion of the composition. Alternatively, immunogenic compositions comprising the polynucleotide and the isolated measles virus polypeptide or fragment, variant, or derivative thereof can be provided as a single formulation, comprising both the polynucleotide and the protein, for example, when the polynucleotide and the protein are administered simultaneously. In another alternative, the polynucleotide portion of the composition and the isolated measles virus polypeptide portion of the composition can be provided simultaneously, but in separate formulations.

Compositions comprising at least one polynucleotide comprising one or more nucleic acid fragments, where each nucleic acid fragment is optionally a fragment of a codon-optimized coding region operably encoding a measles virus polypeptide or fragment, variant, or derivative thereof together with one or more isolated measles virus polypeptides or fragments, variants or derivatives thereof (as either a recombinant protein, a purified subunit, a viral vector expressing the protein, or in the form of an inactivated or attenuated measles virus vaccine) will be referred to herein as “combinatorial polynucleotide (e.g., DNA) vaccine compositions” or “single formulation heterologous prime-boost vaccine compositions.”

The compositions of the invention can be univalent, bivalent, trivalent or multivalent. A univalent composition will comprise only one polynucleotide comprising a nucleic acid fragment, where the nucleic acid fragment is optionally a fragment of a codon-optimized coding region encoding a measles virus polypeptide or a fragment, variant, or derivative thereof, and optionally the same measles virus polypeptide or a fragment, variant, or derivative thereof in isolated form. In a single formulation heterologous prime-boost vaccine composition, a univalent composition can include a polynucleotide comprising a nucleic acid fragment, where the nucleic acid fragment is optionally a fragment of a codon-optimized coding region encoding a measles virus polypeptide or a fragment, variant, or derivative thereof and an isolated polypeptide having the same antigenic region as the polynucleotide. A bivalent composition will comprise, either in polynucleotide or protein form, two different measles virus polypeptides or fragments, variants, or derivatives thereof, each capable of eliciting an immune response. The polynucleotide(s) of the composition can encode two measles virus polypeptides or alternatively, the polynucleotide can encode only one measles virus polypeptide and the second measles virus polypeptide would be provided by an isolated measles virus polypeptide of the invention as in, for example, a single formulation heterologous prime-boost vaccine composition. In the case where both measles virus polypeptides of a bivalent composition are delivered in polynucleotide form, the nucleic acid fragments operably encoding those measles virus polypeptides need not be on the same polynucleotide, but can be on two different polynucleotides. A trivalent or further multivalent composition will comprise three or more measles virus polypeptides or fragments, variants or derivatives thereof, either in isolated form or encoded by one or more polynucleotides of the invention.

The present invention further provides plasmids and other polynucleotide constructs for delivery of nucleic acid fragments of the invention to a vertebrate, e.g., a human, which provide expression of measles virus polypeptides, or fragments, variants, or derivatives thereof. The present invention further provides carriers, excipients, transfection-facilitating agents, immunogenicity-enhancing agents, e.g., adjuvants, or other agent or agents to enhance the transfection, expression or efficacy of the administered gene and its gene product.

In one embodiment, a multivalent composition comprises a single polynucleotide, e.g., plasmid, comprising one or more nucleic acid regions operably encoding measles virus polypeptides or fragments, variants, or derivatives thereof. Reducing the number of polynucleotides, e.g., plasmids in the compositions of the invention can have significant impacts on the manufacture and release of product, thereby reducing the costs associated with manufacturing the compositions. There are a number of approaches to include more than one expressed antigen coding sequence on a single plasmid. These include, for example, the use of Internal Ribosome Entry Site (IRES) sequences, dual promoters/expression cassettes, and fusion proteins.

The invention also provides methods for enhancing the immune response of a vertebrate to measles virus infection by administering to the tissues of a vertebrate one or more polynucleotides each comprising one or more nucleic acid fragments, where each nucleic acid fragment is optionally a fragment of a codon-optimized coding region encoding a measles virus polypeptide or fragment, variant, or derivative thereof; and optionally administering to the tissues of the vertebrate one or more isolated measles virus polypeptides, or fragments, variants, or derivatives thereof. The isolated measles virus polypeptide can be administered prior to, at the same time (simultaneously), or subsequent to administration of the polynucleotides encoding measles virus polypeptides.

In addition, the invention provides consensus amino acid sequences for measles virus polypeptides, or fragments, variants or derivatives thereof, including, but not limited to the HA, or F proteins or fragments, variants or derivatives thereof. Polynucleotides which encode the consensus polypeptides or fragments, variants or derivatives thereof, are also embodied in this invention. Such polynucleotides can be obtained by known methods, for example by backtranslation of the amino acid sequence and PCR synthesis of the corresponding polynucleotide as described below.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 Effect of Vaxfectin® formulation and codon optimization on immune response of mice to DNA expressing MV HA and F. Groups of 6 BALB/c mice were immunized with 1, 3, 10, 15, 30 or 100 μg DNA encoding MV HA and F with Vaxfectin® (VR-HA, VR-F, VR-HA+F), 100 μg DNA without Vaxfectin® (pGAT-HA+F) or 100 μg empty vector with Vaxfectin® (VR) and boosted 4 weeks later (arrow). (A) Time course of the development of MV-specific EIA antibody through 26 weeks after immunization for 30 μg and 100 μg VR-HA+F expressing codon-optimized MV sequences (VR-30, VR-100) compared to 30 μg VR-HA+F (VR/non-30) and 100 μg pGAT-HA+F (pGAT/non-100) expressing non-optimized (non) MV sequences. (B) Peak IgG titers for each of the groups. Mean and SD of the EIA unit (EU) values are shown. (C) Peak neutralizing antibody titers of pooled sera for each of the groups. (D) HA (filled) and F (open or striped)-specific IFN-γ responses of spleen cells measured 4 weeks after vaccination by ELISPOT. Mean spot-forming cells (SFC) per million spleen cells +/−SD are shown.

FIG. 2. Immune responses of rhesus macaques to Vaxfectin®-formulated DNAs expressing HA and F. Groups of five juvenile monkeys or four infant monkeys were immunized with 1 mg of VR-HA+F intramuscularly (IM) or 500 μg of VR-HA+F intradermally (ID) and boosted 4 weeks later (arrow). One infant monkey died of unrelated causes 10 weeks after immunization. (A) MV-specific neutralizing antibodies were measured by plaque reduction. The protective level of neutralizing antibodies is shown with a solid line. Data are presented as the geometric mean of mIU/mL +/−SEM. (B) MV-specific IgG was measured by EIA. Data are expressed as OD values +/−SEM for plasma diluted 1:400. (C) HA-specific and (D) F-specific T cell responses to pooled peptides were measured by IFN-γ ELISPOT assays. (E) Peak HA-specific IFN-γ and IL-4 T cell responses. Data are presented as mean spot-forming cells (SFCs) per million PBMCs +/−SEM.

FIG. 3. Protection from wild-type MV challenge. Thirteen vaccinated juvenile and infant and two unvaccinated control monkeys were challenged 12-15 months after vaccination. (A) Viremia was measured by coculture of serially diluted PBMCs with B95-8 cells. Mean syncytia-forming cells per million PBMCs +/−SEM are shown. (B) MV-specific IgM was measured by EIA and reported as mean optical density +/−SEM for plasma diluted 1:100-200.

FIG. 4. Antibody responses after challenge. MV-specific neutralizing antibody measured by plaque reduction on Vero cells (A) and MV-specific IgG measured by EIA (1:400) (B) are shown. The avidity of MV-specific IgG was assayed by NH₄SCN treatment (C). The avidity index is the concentration of NH₄SCN required to remove 50% of the bound IgG.

FIG. 5. T-cell responses after challenge. MV HA (A) and F (B) specific IFN-γ responses were assayed by ELISPOT. The mean numbers of spot forming cells (SFC) per million PBMC minus the medium control +/−SEM are shown.

FIG. 6. is a schematic representation of VR-HA, that is, a pDNA encoding measles HA antigen.

FIG. 7 is a schematic representation of VR-F, that is, a pDNA encoding measles F antigen.

### DETAILED DESCRIPTION OF THE INVENTION

The present invention relates to methods and compositions which may be used to immunize infant mammals against a measles target antigen, wherein an immunogenically effective amount of a formulated nucleic acid encoding a relevant epitope of a desired target antigen is administered in conjunction with an adjuvant to the infant. It is based, at least in part, on the discovery that such genetic immunization of infant mammals could give rise to effective cellular (including the induction of cytotoxic T lymphocytes) and humoral immune responses against target antigen. Moreover, the present invention may reduce the need for subsequent boost administrations (as are generally required for protein and killed pathogen vaccines), and may prevent side-effects associated with live attenuated vaccines. For instance, using traditional live attenuated virus vaccines, the World Health Organization recommends waiting nine months after birth before immunizing against measles in order to generate an effective immune response. In addition to concern over the immune response, there is a need to avoid undesirable side effects associated with vaccination against these diseases prior to the recommended ages.

The present invention provides for a method for immunizing an infant mammal against measles, comprising inoculating the mammal with an effective amount of a nucleic acid encoding a relevant epitope of the measles virus formulated with an adjuvant. One class of adjuvant that may be used in the present invention is a cationic lipid. In particular the cationic lipid such as but not limited to Vaxfectin® may be used. Vaxfectin® is a recently introduced adjuvant for DNA vaccines that consists of an equimolar mixture of the cationic lipid GAP-DMORIE [(+)-N-(3-aminopropyl)-N,N-dimethyl-2,3-bis(cis-9-tetradecenyloxy)-1-propanaminium bromide)] and a neutral colipid DPyPE (1,2-diphytanoyl-sn-glydero-3-phosphoethanolamine) In particular the present invention discloses the use of Vaxfectin®-formulated plasmid DNAs expressing codon-optimized HA and F as a potential measles vaccine. Vaxfectin® improved antibody and T cell responses to MV in mice. Surprisingly, the Vaxfectin®-formulated DNA vaccine induced sustained production of neutralizing antibodies in both juvenile and infant monkeys after two intramuscular or intradermal injections. More than a year after vaccination, all monkeys were completely protected against rash and viremia when challenged with wild type MV.

The term “infant”, as used herein, refers to a human or non-human mammal during the period of life following birth wherein the immune system has not yet fully matured. In humans, this period extends from birth to the age of about nine months. In mice, this period extends from birth to about four weeks of age. The terms “newborn” and “neonate” refer to a subset of infant mammals, which have essentially just been born. Other characteristics associated with “infants” according to the invention include, an immune response which has: (i) susceptibility to high-zone tolerance (deletion/anergy of T cell precursors, increased tendency to apoptosis); (ii) a Th2 biased helper response (phenotypical particularities of neonatal T cells; decreased CD40L expression on neonatal T cells); (iii) reduced magnitude of the cellular response (reduced number of functional T cells; reduced antigen-presenting cell function); and (iv) reduced magnitude and restricted isotope of humoral response (predominance of IgM^{high}IgD^{low} B cells, reduced cooperation between Th and B cells).

In specific nonlimiting embodiments of the invention, nucleic acid immunization may be administered to an infant animal wherein maternal antibodies remain present in detectable amounts. In a related embodiment, the pregnant mother may be immunized with a nucleic acid-based vaccine prior to delivery so as to increase the level of maternal antibodies passively transferred to the fetus.

The present invention is directed to compositions and methods for enhancing the immune response of a vertebrate in need of protection against measles virus infection by administering in vivo, into a tissue of a vertebrate, at least one polynucleotide comprising one or more nucleic acid fragments, where each nucleic acid fragment is optionally a fragment of a codon-optimized coding region operably encoding a measles virus polypeptide, or a fragment, variant, or derivative thereof in cells of the vertebrate in need of protection. The present invention is also directed to administering in vivo, into a tissue of the vertebrate the above described polynucleotide and at least one isolated measles virus polypeptide, or a fragment, variant, or derivative thereof. The isolated measles virus polypeptide or fragment, variant, or derivative thereof can be, for example, a recombinant protein, a purified subunit protein, a protein expressed and carried by a heterologous live or inactivated or attenuated viral vector expressing the protein, or can be attenuated measles virus, such as those present in conventional, commercially available, live measles virus vaccines. According to either method, the polynucleotide is incorporated into the cells of the vertebrate in vivo, and an immunologically effective amount of the measles protein, or fragment or variant encoded by the polynucleotide is produced in vivo. The isolated protein or fragment, variant, or derivative thereof is also administered in an immunologically effective amount. The polynucleotide can be administered to the vertebrate in need thereof either prior to, at the same time (simultaneously), or subsequent to the administration of the isolated measles virus polypeptide or fragment, variant, or derivative thereof.

Non-limiting examples of measles virus polypeptides within the scope of the invention include, but are not limited to, HA, or F polypeptides, and fragments, derivatives, and variants thereof. Nucleotide and amino acid sequences of measles virus polypeptides from a wide variety of measles virus types and subtypes are known in the art. The nucleotide sequences set out below are the wild-type sequences. For example, the nucleotide sequence of the F protein is available as GenBank Accession Number AF266287, referred to herein as SEQ ID NO:1.

The nucleotide sequence of the wild type HA protein is available as GenBank Accession Number AF266287, referred to herein as SEQ ID NO:2.

The present invention also provides vaccine compositions and methods for delivery of measles virus coding sequences to a vertebrate with optimal expression and safety conferred through codon optimization and/or other manipulations. These vaccine compositions are prepared and administered in such a manner that the encoded gene products are optimally expressed in the vertebrate of interest. As a result, these compositions and methods are useful in stimulating an immune response against measles virus infection. Also included in the invention are expression systems, delivery systems, and codon-optimized measles virus coding regions.

In a specific embodiment, the invention provides combinatorial polynucleotide (e.g., DNA) vaccines which combine both a polynucleotide vaccine and polypeptide (e.g., either a recombinant protein, a purified subunit protein, a viral vector expressing an isolated measles virus polypeptide, or in the form of an inactivated or attenuated measles virus vaccine) vaccine in a single formulation. The single formulation comprises a measles virus polypeptide-encoding polynucleotide vaccine as described herein, and optionally, an effective amount of a desired isolated measles virus polypeptide or fragment, variant, or derivative thereof. The polypeptide may exist in any form, for example, a recombinant protein, a purified subunit protein, a viral vector expressing an isolated measles virus polypeptide, or in the form of an inactivated or attenuated measles virus vaccine. The measles virus polypeptide or fragment, variant, or derivative thereof encoded by the polynucleotide vaccine may be identical to the isolated measles virus polypeptide or fragment, variant, or derivative thereof. Alternatively, the measles virus polypeptide or fragment, variant, or derivative thereof encoded by the polynucleotide may be different from the isolated measles virus polypeptide or fragment, variant, or derivative thereof.

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

The term “polynucleotide” is intended to encompass a singular nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A polynucleotide may comprise a conventional phosphodiester bond or a non-conventional bond (e.g., an amide bond, such as found in peptide nucleic acids (PNA)).

The terms “nucleic acid” or “nucleic acid fragment” refer to any one or more nucleic acid segments, e.g., DNA or RNA fragments, present in a polynucleotide or construct. A nucleic acid or fragment thereof may be provided in linear (e.g., mRNA) or circular (e.g., plasmid) form as well as double-stranded or single-stranded forms. By “isolated” nucleic acid or polynucleotide is intended a nucleic acid molecule, DNA or RNA, which has been removed from its native environment. For example, a recombinant polynucleotide contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of the polynucleotides of the present invention. Isolated polynucleotides or nucleic acids according to the present invention further include such molecules produced synthetically.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, and the like, are not part of a coding region. Two or more nucleic acids or nucleic acid fragments of the present invention can be present in a single polynucleotide construct, e.g., on a single plasmid, or in separate polynucleotide constructs, e.g., on separate (different) plasmids. Furthermore, any nucleic acid or nucleic acid fragment may encode a single measles virus polypeptide or fragment, derivative, or variant thereof, e.g., or may encode more than one polypeptide, e.g., a nucleic acid may encode two or more polypeptides. In addition, a nucleic acid may include a regulatory element such as a promoter, ribosome binding site, or a transcription terminator, or may encode heterologous coding regions fused to the measles virus coding region, e.g., specialized elements or motifs, such as a secretory signal peptide or a heterologous functional domain.

The terms “fragment,” “variant,” “derivative” and “analog” when referring to measles virus polypeptides of the present invention include any polypeptides which retain at least some of the immunogenicity or antigenicity of the corresponding native polypeptide. Fragments of measles virus polypeptides of the present invention include proteolytic fragments, deletion fragments and in particular, fragments of measles virus polypeptides which exhibit increased secretion from the cell or higher immunogenicity or reduced pathogenicity when delivered to an animal. Polypeptide fragments further include any portion of the polypeptide which comprises an antigenic or immunogenic epitope of the native polypeptide, including linear as well as three-dimensional epitopes. Variants of measles virus polypeptides of the present invention include fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome or genome of an organism or virus. Genes II, (Lewin, B., ed., John Wiley & Sons, New York (1985)). For example, as used herein, variations in a given gene product. When referring to measles virus F or HA proteins, each such protein is a “variant,” in that native measles virus strains are distinguished by the type of F and HA proteins encoded by the virus. However, within a single HA or F variant type, further naturally or non-naturally occurring variations such as amino acid deletions, insertions or substitutions may occur. Non-naturally occurring variants may be produced using art-known mutagenesis techniques. Variant polypeptides may comprise conservative or non-conservative amino acid substitutions, deletions or additions. Derivatives of measles virus polypeptides of the present invention, are polypeptides which have been altered so as to exhibit additional features not found on the native polypeptide. Examples include fusion proteins. An analog is another form of a measles virus polypeptide of the present invention. An example is a proprotein which can be activated by cleavage of the proprotein to produce an active mature polypeptide.

The terms “infectious polynucleotide” or “infectious nucleic acid” are intended to encompass isolated viral polynucleotides and/or nucleic acids which are solely sufficient to mediate the synthesis of complete infectious virus particles upon uptake by permissive cells. Thus, “infectious nucleic acids” do not require pre-synthesized copies of any of the polypeptides it encodes, e.g., viral replicases, in order to initiate its replication cycle in a permissive host cell.

The terms “non-infectious polynucleotide” or “non-infectious nucleic acid” as defined herein are polynucleotides or nucleic acids which cannot, without additional added materials, e.g., polypeptides, mediate the synthesis of complete infectious virus particles upon uptake by permissive cells. An infectious polynucleotide or nucleic acid is not made “non-infectious” simply because it is taken up by a non-permissive cell. For example, an infectious viral polynucleotide from a virus with limited host range is infectious if it is capable of mediating the synthesis of complete infectious virus particles when taken up by cells derived from a permissive host (i.e., a host permissive for the virus itself). The fact that uptake by cells derived from a non-permissive host does not result in the synthesis of complete infectious virus particles does not make the nucleic acid “non-infectious.” In other words, the term is not qualified by the nature of the host cell, the tissue type, or the species taking up the polynucleotide or nucleic acid fragment.

In some cases, an isolated infectious polynucleotide or nucleic acid may produce fully-infectious virus particles in a host cell population which lacks receptors for the virus particles, i.e., is non-permissive for virus entry. Thus viruses produced will not infect surrounding cells. However, if the supernatant containing the virus particles is transferred to cells which are permissive for the virus, infection will take place.

The terms “replicating polynucleotide” or “replicating nucleic acid” are meant to encompass those polynucleotides and/or nucleic acids which, upon being taken up by a permissive host cell, are capable of producing multiple, e.g., one or more copies of the same polynucleotide or nucleic acid. Infectious polynucleotides and nucleic acids are a subset of replicating polynucleotides and nucleic acids; the terms are not synonymous. For example, a defective virus genome lacking the genes for virus coat proteins may replicate, e.g., produce multiple copies of itself, but is not infectious because it is incapable of mediating the synthesis of complete infectious virus particles unless the coat proteins, or another nucleic acid encoding the coat proteins, are exogenously provided.

In certain embodiments, the polynucleotide, nucleic acid, or nucleic acid fragment is DNA. In the case of DNA, a polynucleotide comprising a nucleic acid which encodes a polypeptide normally also comprises a promoter and/or other transcription or translation control elements operably associated with the polypeptide-encoding nucleic acid fragment. An operable association is when a nucleic acid fragment encoding a gene product, e.g., a polypeptide, is associated with one or more regulatory sequences in such a way as to place expression of the gene product under the influence or control of the regulatory sequence(s). Two DNA fragments (such as a polypeptide-encoding nucleic acid fragment and a promoter associated with the 5′ end of the nucleic acid fragment) are “operably associated” if induction of promoter function results in the transcription of mRNA encoding the desired gene product and if the nature of the linkage between the two DNA fragments does not (1) result in the introduction of a frame-shift mutation, (2) interfere with the ability of the expression regulatory sequences to direct the expression of the gene product, or (3) interfere with the ability of the DNA template to be transcribed. Thus, a promoter region would be operably associated with a nucleic acid fragment encoding a polypeptide if the promoter was capable of effecting transcription of that nucleic acid fragment. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, elements from picornaviruses (particularly an internal ribosome entry site, or IRES, also referred to as a CITE sequence).

A DNA polynucleotide of the present invention may be a circular or linearized plasmid or vector, or other linear DNA which may also be non-infectious and nonintegrating (i.e., does not integrate into the genome of vertebrate cells). A linearized plasmid is a plasmid that was previously circular but has been linearized, for example, by digestion with a restriction endonuclease. Linear DNA may be advantageous in certain situations as discussed, e.g., in Cherng, J. Y., et al., J. Control. Release 60:343-53 (1999), and Chen, Z. Y., et al. Mol. Ther. 3:403-10 (2001). As used herein, the terms plasmid and vector can be used interchangeably.

Alternatively, DNA virus genomes may be used to administer DNA polynucleotides into vertebrate cells. In certain embodiments, a DNA virus genome of the present invention is nonreplicative, noninfectious, and/or nonintegrating. Suitable DNA virus genomes include without limitation, herpesvirus genomes, adenovirus genomes, adeno-associated virus genomes, and poxvirus genomes. References citing methods for the in vivo introduction of non-infectious virus genomes to vertebrate tissues are well known to those of ordinary skill in the art.

In other embodiments, a polynucleotide of the present invention is RNA, for example, in the form of messenger RNA (mRNA). Methods for introducing RNA sequences into vertebrate cells are described in U.S. Pat. No. 5,580,859.

Polynucleotides, nucleic acids, and nucleic acid fragments of the present invention may be associated with additional nucleic acids which encode secretory or signal peptides, which direct the secretion of a polypeptide encoded by a nucleic acid fragment or polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native leader sequence is used, or a functional derivative of that sequence that retains the ability to direct the secretion of the polypeptide that is operably associated with it. Alternatively, a heterologous mammalian leader sequence, or a functional derivative thereof, may be used. For example, the wild-type leader sequence may be substituted with the leader sequence of human tissue plasminogen activator (TPA) or mouse β-glucuronidase.

In accordance with one aspect of the present invention, there is provided a polynucleotide construct, for example, a plasmid, comprising a nucleic acid fragment, where the nucleic acid fragment is a fragment of a codon-optimized coding region operably encoding a measles virus-derived polypeptide, where the coding region is optimized for expression in vertebrate cells, of a desired vertebrate species, e.g., humans, to be delivered to a vertebrate to be treated or immunized. Suitable measles virus polypeptides, or fragments, variants, or derivatives thereof may be derived from, but are not limited to, the measles virus HA, or F proteins. Additional measles virus-derived coding sequences, may also be included on the plasmid, or on a separate plasmid, and expressed, either using native measles virus codons or codons optimized for expression in the vertebrate to be treated or immunized. When such a plasmid encoding one or more optimized measles sequences is delivered, in vivo to a tissue of the vertebrate to be treated or immunized, one or more of the encoded gene products will be expressed, i.e., transcribed and translated. The level of expression of the gene product(s) will depend to a significant extent on the strength of the associated promoter and the presence and activation of an associated enhancer element, as well as the degree of optimization of the coding region.

As used herein, the term “plasmid” refers to a construct made up of genetic material (i.e., nucleic acids). Typically a plasmid contains an origin of replication which is functional in bacterial host cells, e.g., Escherichia coli, and selectable markers for detecting bacterial host cells comprising the plasmid. Plasmids of the present invention may include genetic elements as described herein arranged such that an inserted coding sequence can be transcribed and translated in eukaryotic cells. Also, the plasmid may include a sequence from a viral nucleic acid. However, such viral sequences normally are not sufficient to direct or allow the incorporation of the plasmid into a viral particle, and the plasmid is therefore a non-viral vector. In certain embodiments described herein, a plasmid is a closed circular DNA molecule.

The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” can be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

Also included as polypeptides of the present invention are fragments, derivatives, analogs, or variants of the foregoing polypeptides, and any combination thereof. Polypeptides, and fragments, derivatives, analogs, or variants thereof of the present invention can be antigenic and immunogenic polypeptides related to measles virus polypeptides, which are used to prevent or treat, i.e., cure, ameliorate, lessen the severity of, or prevent or reduce contagion of infectious disease caused by the measles virus.

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic. It is quite likely that an immunogenic polypeptide will also be antigenic, but an antigenic polypeptide, because of its size or conformation, may not necessarily be immunogenic. Examples of antigenic and immunogenic polypeptides of the present invention include, but are not limited to, e.g., HA, or F or fragments or variants thereof, or any of the foregoing polypeptides or fragments fused to a heterologous polypeptide, for example, a hepatitis B core antigen. Isolated antigenic and immunogenic polypeptides of the present invention in addition to those encoded by polynucleotides of the invention, may be provided as a recombinant protein, a purified subunit, a viral vector expressing the protein, or may be provided in the form of whole measles virus vaccine, e.g., a live-attenuated virus vaccine, a heat-killed virus vaccine, etc.

Immunospecific binding excludes non-specific binding but does not exclude cross-reactivity with other antigens. Where all immunogenic epitopes are antigenic, antigenic epitopes need not be immunogenic.

By an “isolated” measles virus polypeptide or a fragment, variant, or derivative thereof is intended a measles virus polypeptide or protein that is not in its natural form. No particular level of purification is required. For example, an isolated measles virus polypeptide can be removed from its native or natural environment. Recombinantly produced measles virus polypeptides and proteins expressed in host cells are considered isolated for purposes of the invention, as are native or recombinant measles virus polypeptides which have been separated, fractionated, or partially or substantially purified by any suitable technique, including the separation of measles virus virions from eggs or culture cells in which they have been propagated. In addition, an isolated measles virus polypeptide or protein can be provided as a live or inactivated viral vector expressing an isolated measles virus polypeptide and can include those found in measles virus vaccine compositions. Thus, isolated measles virus polypeptides and proteins can be provided as, for example, recombinant measles virus polypeptides, a purified subunit of measles virus, a viral vector expressing an isolated measles virus polypeptide, or in the form of an inactivated or attenuated measles virus vaccine.

The term “epitopes,” as used herein, refers to portions of a polypeptide having antigenic or immunogenic activity in a vertebrate, for example a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind as determined by any method well known in the art.

The term “immunogenic carrier” as used herein refers to a first polypeptide or fragment, variant, or derivative thereof which enhances the immunogenicity of a second polypeptide or fragment, variant, or derivative thereof. Typically, an “immunogenic carrier” is fused to or conjugated to the desired polypeptide or fragment thereof. An example of an “immunogenic carrier” is a recombinant hepatitis B core antigen expressing, as a surface epitope, an immunogenic epitope of interest. See, e.g., European Patent No. EP 0385610 B 1.

In the present invention, antigenic epitopes preferably contain a sequence of at least 4, at least 5, at least 6, at least 7, at least 8, at least 9, at least 10, at least 15, at least 20, at least 25, or between about 8 to about 30 amino acids contained within the amino acid sequence of a measles virus polypeptide of the invention, e.g., an HA polypeptide, or an F polypeptide. Certain polypeptides comprising immunogenic or antigenic epitopes are at least 5, 10, 15, 20, 25, 30, 35, 40, 45, 50, 55, 60, 65, 70, 75, 80, 85, 90, 95, or 100 amino acid residues in length. Antigenic as well as immunogenic epitopes may be linear, i.e., be comprised of contiguous amino acids in a polypeptide, or may be three dimensional, i.e., where an epitope is comprised of non-contiguous amino acids which come together due to the secondary or tertiary structure of the polypeptide, thereby forming an epitope.

As to the selection of peptides or polypeptides bearing an antigenic epitope (e.g., that contain a region of a protein molecule to which an antibody or T cell receptor can bind), it is well known in that art that relatively short synthetic peptides that mimic part of a protein sequence are routinely capable of eliciting an antiserum that reacts with the partially mimicked protein. See, e.g., Sutcliffe, J. G., et al., Science 219:660-666 (1983).

Peptides capable of eliciting an immunogenic response are frequently represented in the primary sequence of a protein, can be characterized by a set of simple chemical rules, and are confined neither to immunodominant regions of intact proteins nor to the amino or carboxyl terminals. Peptides that are extremely hydrophobic and those of six or fewer residues generally are ineffective at inducing antibodies that bind to the mimicked protein; longer peptides, especially those containing proline residues, usually are effective. Sutcliffe et al., supra, at 661.

### Codon Optimization

“Codon optimization” is defined as modifying a nucleic acid sequence for enhanced expression in the cells of the vertebrate of interest, e.g. human, by replacing at least one, more than one, or a significant number, of codons of the native sequence with codons that are more frequently or most frequently used in the genes of that vertebrate. Various species exhibit particular bias for certain codons of a particular amino acid.

In one aspect, the present invention relates to polynucleotides comprising nucleic acid fragments of codon-optimized coding regions which encode measles virus polypeptides, or fragments, variants, or derivatives thereof, with the codon usage adapted for optimized expression in the cells of a given vertebrate, e.g., humans. These polynucleotides are prepared by incorporating codons preferred for use in the genes of the vertebrate of interest into the DNA sequence. Also provided are polynucleotide expression constructs, vectors, and host cells comprising nucleic acid fragments of codon-optimized coding regions which encode measles virus polypeptides, and fragments, variants, or derivatives thereof, and various methods of using the polynucleotide expression constructs, vectors, host cells to treat or prevent measles disease in a vertebrate.

As used herein the term “codon-optimized coding region” means a nucleic acid coding region that has been adapted for expression in the cells of a given vertebrate by replacing at least one, or more than one, or a significant number, of codons with one or more codons that are more frequently used in the genes of that vertebrate.

Deviations in the nucleotide sequence that comprise the codons encoding the amino acids of any polypeptide chain allow for variations in the sequence coding for the gene. Because each codon consists of three nucleotides, and the nucleotides comprising DNA are restricted to four specific bases, there are 64 possible combinations of nucleotides, 61 of which encode amino acids (the remaining three codons encode signals ending translation (stop or termination)). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 1
The Standard Genetic Code

| | T(U) | C | A | G |
|---|---|---|---|---|
| T(U) | TTT Phe (F) | TCT Ser (S) | TAT Tyr (Y) | TGT Cys (C) |
| | TTC Phe | TCC Ser | TAC Tyr | TGC Cys |
| | TTA Leu (L) | TCA Ser | TAA Ter | TGA Ter |
| | TTG Leu | TCG Ser | TAG Ter | TGG Trp (W) |
| C | CTT Leu (L) | CCT Pro (P) | CAT His (H) | CGT Arg (R) |
| | CTC Leu | CCC Pro | CAC His | CGC Arg |
| | CTA Leu | CCA Pro | CAA Gln (Q) | CGA Arg |
| | CTG Leu | CCG Pro | CAG Gln | CGG Arg |
| A | ATT Ile (I) | ACT Thr (T) | AAT Asn (N) | AGT Ser (S) |
| | ATC Ile | ACC Thr | AAC Asn | AGC Ser |
| | ATA Ile | ACA Thr | AAA Lys (K) | AGA Arg (R) |
| | ATG Met (M) | ACG Thr | AAG Lys | AGG Arg |
| G | GTT Val (V) | GCT Ala (A) | GAT Asp (D) | GGT Gly (G) |
| | GTC Val | GCC Ala | GAC Asp | GGC Gly |
| | GTA Val | GCA Ala | GAA Glu (E) | GGA Gly |
| | GTG Val | GCG Ala | GAG Glu | GGG Gly |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (Jul. 9, 2002), and these tables can be adapted in a number of ways. See Nakamura, Y., et al., “Codon usage tabulated from the international DNA sequence databases: status for the year 2000” Nucl. Acids Res. 28:292 (2000). As examples, the codon usage tables for human, mouse, domestic cat, and cow, calculated from GenBank Release 128.0 (15 Feb. 2002), are reproduced below as Tables 2-5. These Tables use mRNA nomenclature, and so instead of thymine (T) which is found in DNA, the Tables use uracil (U) which is found in RNA. The Tables have been adapted so that frequencies are calculated for each amino acid, rather than for all 64 codons.

TABLE 2
Codon Usage Table for Human Genes
(Homo sapiens)

| Amino | | | |
|---|---|---|---|
| Acid | Codon | Number | Frequency |
| Phe | UUU | 326146 | 0.4525 |
| Phe | UUC | 394680 | 0.5475 |
| Total | | 720826 | |
| Leu | UUA | 139249 | 0.0728 |
| Leu | UUG | 242151 | 0.1266 |
| Leu | CUU | 246206 | 0.1287 |
| Leu | CUC | 374262 | 0.1956 |
| Leu | CUA | 133980 | 0.0700 |
| Leu | CUG | 777077 | 0.4062 |
| Total | | 1912925 | |
| Ile | AUU | 303721 | 0.3554 |
| Ile | AUC | 414483 | 0.4850 |
| Ile | AUA | 136399 | 0.1596 |
| Total | | 854603 | |
| Met | AUG | 430946 | 1.0000 |
| Total | | 430946 | |
| Val | GUU | 210423 | 0.1773 |
| Val | GUC | 282445 | 0.2380 |
| Val | GUA | 134991 | 0.1137 |
| Val | GUG | 559044 | 0.4710 |
| Total | | 1186903 | |
| Ser | UCU | 282407 | 0.1840 |
| Ser | UCC | 336349 | 0.2191 |
| Ser | UCA | 225963 | 0.1472 |
| Ser | UCG | 86761 | 0.0565 |
| Ser | AGU | 230047 | 0.1499 |
| Ser | AGC | 373362 | 0.2433 |
| Total | | 1534889 | |
| Pro | CCU | 333705 | 0.2834 |
| Pro | CCC | 386462 | 0.3281 |
| Pro | CCA | 322220 | 0.2736 |
| Pro | CCG | 135317 | 0.1149 |
| Total | | 1177704 | |
| Thr | ACU | 247913 | 0.2419 |
| Thr | ACC | 371420 | 0.3624 |
| Thr | ACA | 285655 | 0.2787 |
| Thr | ACG | 120022 | 0.1171 |
| Total | | 1025010 | |
| Ala | GCU | 360146 | 0.2637 |
| Ala | GCC | 551452 | 0.4037 |
| Ala | GCA | 308034 | 0.2255 |
| Ala | GCG | 146233 | 0.1071 |
| Total | | 1365865 | |
| Tyr | UAU | 232240 | 0.4347 |
| Tyr | UAC | 301978 | 0.5653 |
| Total | | 534218 | |
| His | CAU | 201389 | 0.4113 |
| His | CAC | 288200 | 0.5887 |
| Total | | 489589 | |
| Gln | CAA | 227742 | 0.2541 |
| Gln | CAG | 668391 | 0.7459 |
| Total | | 896133 | |
| Asn | AAU | 322271 | 0.4614 |
| Asn | AAC | 376210 | 0.5386 |
| Total | | 698481 | |
| Lys | AAA | 462660 | 0.4212 |
| Lys | AAG | 635755 | 0.5788 |
| Total | | 1098415 | |
| Asp | GAU | 430744 | 0.4613 |
| Asp | GAC | 502940 | 0.5387 |
| Total | | 933684 | |
| Glu | GAA | 561277 | 0.4161 |
| Glu | GAG | 787712 | 0.5839 |
| Total | | 1348989 | |
| Cys | UGU | 190962 | 0.4468 |
| Cys | UGC | 236400 | 0.5532 |
| Total | | 427362 | |
| Trp | UGG | 248083 | 1.0000 |
| Total | | 248083 | |
| Arg | CGU | 90899 | 0.0830 |
| Arg | CGC | 210931 | 0.1927 |
| Arg | CGA | 122555 | 0.1120 |
| Arg | CGG | 228970 | 0.2092 |
| Arg | AGA | 221221 | 0.2021 |
| Arg | AGG | 220119 | 0.2011 |
| Total | | 1094695 | |
| Gly | GGU | 209450 | 0.1632 |
| Gly | GGC | 441320 | 0.3438 |
| Gly | GGA | 315726 | 0.2459 |
| Gly | GGG | 317263 | 0.2471 |
| Total | | 1283759 | |
| Stop | UAA | 13963 | |
| Stop | UAG | 10631 | |
| Stop | UGA | 24607 | |

TABLE 3
Codon Usage Table for Mouse Genes
(Mus musculus)

| Amino | | | |
|---|---|---|---|
| Acid | Codon | Number | Frequency |
| Phe | UUU | 150467 | 0.4321 |
| Phe | UUC | 197795 | 0.5679 |
| Total | | 348262 | |
| Leu | UUA | 55635 | 0.0625 |
| Leu | UUG | 116210 | 0.1306 |
| Leu | CUU | 114699 | 0.1289 |
| Leu | CUC | 179248 | 0.2015 |
| Leu | CUA | 69237 | 0.0778 |
| Leu | CUG | 354743 | 0.3987 |
| Total | | 889772 | |
| Ile | AUU | 137513 | 0.3367 |
| Ile | AUC | 208533 | 0.5106 |
| Ile | AUA | 62349 | 0.1527 |
| Total | | 408395 | |
| Met | AUG | 204546 | 1.0000 |
| Total | | 204546 | |
| Val | GUU | 93754 | 0.1673 |
| Val | GUC | 140762 | 0.2513 |
| Val | GUA | 64417 | 0.1150 |
| Val | GUG | 261308 | 0.4664 |
| Total | | 560241 | |
| Ser | UCU | 139576 | 0.1936 |
| Ser | UCC | 160313 | 0.2224 |
| Ser | UCA | 100524 | 0.1394 |
| Ser | UCG | 38632 | 0.0536 |
| Ser | AGU | 108413 | 0.1504 |
| Ser | AGC | 173518 | 0.2407 |
| Total | | 720976 | |
| Pro | CCU | 162613 | 0.3036 |
| Pro | CCC | 164796 | 0.3077 |
| Pro | CCA | 151091 | 0.2821 |
| Pro | CCG | 57032 | 0.1065 |
| Total | | 535532 | |
| Thr | ACU | 119832 | 0.2472 |
| Thr | ACC | 172415 | 0.3556 |
| Thr | ACA | 140420 | 0.2896 |
| Thr | ACG | 52142 | 0.1076 |
| Total | | 484809 | |
| Ala | GCU | 178593 | 0.2905 |
| Ala | GCC | 236018 | 0.3839 |
| Ala | GCA | 139697 | 0.2272 |
| Ala | GCG | 60444 | 0.0983 |
| Total | | 614752 | |
| Tyr | UAU | 108556 | 0.4219 |
| Tyr | UAC | 148772 | 0.5781 |
| Total | | 257328 | |
| His | CAU | 88786 | 0.3973 |
| His | CAC | 134705 | 0.6027 |
| Total | | 223491 | |
| Gln | CAA | 101783 | 0.2520 |
| Gln | CAG | 302064 | 0.7480 |
| Total | | 403847 | |
| Asn | AAU | 138868 | 0.4254 |
| Asn | AAC | 187541 | 0.5746 |
| Total | | 326409 | |
| Lys | AAA | 188707 | 0.3839 |
| Lys | AAG | 302799 | 0.6161 |
| Total | | 491506 | |
| Asp | GAU | 189372 | 0.4414 |
| Asp | GAC | 239670 | 0.5586 |
| Total | | 429042 | |
| Glu | GAA | 235842 | 0.4015 |
| Glu | GAG | 351582 | 0.5985 |
| Total | | 587424 | |
| Cys | UGU | 97385 | 0.4716 |
| Cys | UGC | 109130 | 0.5284 |
| Total | | 206515 | |
| Trp | UGG | 112588 | 1.0000 |
| Total | | 112588 | |
| Arg | CGU | 41703 | 0.0863 |
| Arg | CGC | 86351 | 0.1787 |
| Arg | CGA | 58928 | 0.1220 |
| Arg | CGG | 92277 | 0.1910 |
| Arg | AGA | 101029 | 0.2091 |
| Arg | AGG | 102859 | 0.2129 |
| Total | | 483147 | |
| Gly | GGU | 103673 | 0.1750 |
| Gly | GGC | 198604 | 0.3352 |
| Gly | GGA | 151497 | 0.2557 |
| Gly | GGG | 138700 | 0.2341 |
| Total | | 592474 | |
| Stop | UAA | 5499 | |
| Stop | UAG | 4661 | |
| Stop | UGA | 10356 | |

TABLE 4
Codon Usage Table for Domestic Cat Genes
(Felis cattus)

| Amino | | | Frequency |
|---|---|---|---|
| Acid | Codon | Number | of usage |
| Phe | UUU | 1204.00 | 0.4039 |
| Phe | UUC | 1777.00 | 0.5961 |
| Total | | 2981 | |
| Leu | UUA | 404.00 | 0.0570 |
| Leu | UUG | 857.00 | 0.1209 |
| Leu | CUU | 791.00 | 0.1116 |
| Leu | CUC | 1513.00 | 0.2135 |
| Leu | CUA | 488.00 | 0.0688 |
| Leu | CUG | 3035.00 | 0.4282 |
| Total | | 7088 | |
| Ile | AUU | 1018.00 | 0.2984 |
| Ile | AUC | 1835.00 | 0.5380 |
| Ile | AUA | 558.00 | 0.1636 |
| Total | | 3411 | |
| Met | AUG | 1553.00 | 0.0036 |
| Total | | 1553 | |
| Val | GUU | 696.00 | 0.1512 |
| Val | GUC | 1279.00 | 0.2779 |
| Val | GUA | 463.00 | 0.1006 |
| Val | GUG | 2164.00 | 0.4702 |
| Total | | 4602 | |
| Ser | UCU | 940.00 | 0.1875 |
| Ser | UCC | 1260.00 | 0.2513 |
| Ser | UCA | 608.00 | 0.1213 |
| Ser | UCG | 332.00 | 0.0662 |
| Ser | AGU | 672.00 | 0.1340 |
| Ser | AGC | 1202.00 | 0.2397 |
| Total | | 5014 | |
| Pro | CCU | 958.00 | 0.2626 |
| Pro | CCC | 1375.00 | 0.3769 |
| Pro | CCA | 850.00 | 0.2330 |
| Pro | CCG | 465.00 | 0.1275 |
| Total | | 3648 | |
| Thr | ACU | 822.00 | 0.2127 |
| Thr | ACC | 1574.00 | 0.4072 |
| Thr | ACA | 903.00 | 0.2336 |
| Thr | ACG | 566.00 | 0.1464 |
| Total | | 3865 | |
| Ala | GCU | 1129.00 | 0.2496 |
| Ala | GCC | 1951.00 | 0.4313 |
| Ala | GCA | 883.00 | 0.1952 |
| Ala | GCG | 561.00 | 0.1240 |
| Total | | 4524 | |
| Tyr | UAU | 837.00 | 0.3779 |
| Tyr | UAC | 1378.00 | 0.6221 |
| Total | | 2215 | |
| His | CAU | 594.00 | 0.3738 |
| His | CAC | 995.00 | 0.6262 |
| Total | | 1589 | |
| Gln | CAA | 747.00 | 0.2783 |
| Gln | CAG | 1937.00 | 0.7217 |
| Total | | 2684 | |
| Asn | AAU | 1109.00 | 0.3949 |
| Asn | AAC | 1699.00 | 0.6051 |
| Total | | 2808 | |
| Lys | AAA | 1445.00 | 0.4088 |
| Lys | AAG | 2090.00 | 0.5912 |
| Total | | 3535 | |
| Asp | GAU | 1255.00 | 0.4055 |
| Asp | GAC | 1840.00 | 0.5945 |
| Total | | 3095 | |
| Glu | GAA | 1637.00 | 0.4164 |
| Glu | GAG | 2294.00 | 0.5836 |
| Total | | 3931 | |
| Cys | UGU | 719.00 | 0.4425 |
| Cys | UGC | 906.00 | 0.5575 |
| Total | | 1625 | |
| Trp | UGG | 1073.00 | 1.0000 |
| Total | | 1073 | |
| Arg | CGU | 236.00 | 0.0700 |
| Arg | CGC | 629.00 | 0.1865 |
| Arg | CGA | 354.00 | 0.1050 |
| Arg | CGG | 662.00 | 0.1963 |
| Arg | AGA | 712.00 | 0.2112 |
| Arg | AGG | 779.00 | 0.2310 |
| Total | | 3372 | |
| Gly | GGU | 648.00 | 0.1498 |
| Gly | GGC | 1536.00 | 0.3551 |
| Gly | GGA | 1065.00 | 0.2462 |
| Gly | GGG | 1077.00 | 0.2490 |
| Total | | 4326 | |
| Stop | UAA | 55 | |
| Stop | UAG | 36 | |
| Stop | UGA | 110 | |

TABLE 5
Codon Usage Table for Cow Genes
(Bos taurus)

| Amino | | | Frequency |
|---|---|---|---|
| Acid | Codon | Number | of usage |
| Phe | UUU | 13002 | 0.4112 |
| Phe | UUC | 18614 | 0.5888 |
| Total | | 31616 | |
| Leu | UUA | 4467 | 0.0590 |
| Leu | UUG | 9024 | 0.1192 |
| Leu | CUU | 9069 | 0.1198 |
| Leu | CUC | 16003 | 0.2114 |
| Leu | CUA | 4608 | 0.0609 |
| Leu | CUG | 32536 | 0.4298 |
| Total | | 75707 | |
| Ile | AUU | 12474 | 0.3313 |
| Ile | AUC | 19800 | 0.5258 |
| Ile | AUA | 5381 | 0.1429 |
| Total | | 37655 | |
| Met | AUG | 17770 | 1.0000 |
| Total | | 17770 | |
| Val | GUU | 8212 | 0.1635 |
| Val | GUC | 12846 | 0.2558 |
| Val | GUA | 4932 | 0.0982 |
| Val | GUG | 24222 | 0.4824 |
| Total | | 50212 | |
| Ser | UCU | 10287 | 0.1804 |
| Ser | UCC | 13258 | 0.2325 |
| Ser | UCA | 7678 | 0.1347 |
| Ser | UCG | 3470 | 0.0609 |
| Ser | AGU | 8040 | 0.1410 |
| Ser | AGC | 14279 | 0.2505 |
| Total | | 57012 | |
| Pro | CCU | 11695 | 0.2684 |
| Pro | CCC | 15221 | 0.3493 |
| Pro | CCA | 11039 | 0.2533 |
| Pro | CCG | 5621 | 0.1290 |
| Total | | 43576 | |
| Thr | ACU | 9372 | 0.2203 |
| Thr | ACC | 16574 | 0.3895 |
| Thr | ACA | 10892 | 0.2560 |
| Thr | ACG | 5712 | 0.1342 |
| Total | | 42550 | |
| Ala | GCU | 13923 | 0.2592 |
| Ala | GCC | 23073 | 0.4295 |
| Ala | GCA | 10704 | 0.1992 |
| Ala | GCG | 6025 | 0.1121 |
| Total | | 53725 | |
| Tyr | UAU | 9441 | 0.3882 |
| Tyr | UAC | 14882 | 0.6118 |
| Total | | 24323 | |
| His | CAU | 6528 | 0.3649 |
| His | CAC | 11363 | 0.6351 |
| Total | | 17891 | |
| Gln | CAA | 8060 | 0.2430 |
| Gln | CAG | 25108 | 0.7570 |
| Total | | 33168 | |
| Asn | AAU | 12491 | 0.4088 |
| Asn | AAC | 18063 | 0.5912 |
| Total | | 30554 | |
| Lys | AAA | 17244 | 0.3897 |
| Lys | AAG | 27000 | 0.6103 |
| Total | | 44244 | |
| Asp | GAU | 16615 | 0.4239 |
| Asp | GAC | 22580 | 0.5761 |
| Total | | 39195 | |
| Glu | GAA | 21102 | 0.4007 |
| Glu | GAG | 31555 | 0.5993 |
| Total | | 52657 | |
| Cys | UGU | 7556 | 0.4200 |
| Cys | UGC | 10436 | 0.5800 |
| Total | | 17992 | |
| Trp | UGG | 10706 | 1.0000 |
| Total | | 10706 | |
| Arg | CGU | 3391 | 0.0824 |
| Arg | CGC | 7998 | 0.1943 |
| Arg | CGA | 4558 | 0.1108 |
| Arg | CGG | 8300 | 0.2017 |
| Arg | AGA | 8237 | 0.2001 |
| Arg | AGG | 8671 | 0.2107 |
| Total | | 41155 | |
| Gly | GGU | 8508 | 0.1616 |
| Gly | GGC | 18517 | 0.3518 |
| Gly | GGA | 12838 | 0.2439 |
| Gly | GGG | 12772 | 0.2427 |
| Total | | 52635 | |
| Stop | UAA | 555 | |
| Stop | UAG | 394 | |
| Stop | UGA | 392 | |

By utilizing these or similar tables, one of ordinary skill in the art can apply the frequencies to any given polypeptide sequence, and produce a nucleic acid fragment of a codon-optimized coding region which encodes the polypeptide, but which uses codons more optimal for a given species. Codon-optimized coding regions can be designed by various different methods.

In another method, termed “full-optimization,” the actual frequencies of the codons are distributed randomly throughout the coding region. Thus, using this method for optimization, if a hypothetical polypeptide sequence had 100 leucine residues, referring to Table 2 for frequency of usage in humans, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this method; however, the sequence always encodes the same polypeptide.

As an example, a nucleotide sequence for HA protein (SEQ ID NO:2) fully optimized for human codon usage, is shown as SEQ ID NO:4.

In using the “full-optimization” method, an entire polypeptide sequence may be codon-optimized as described above. With respect to various desired fragments, variants or derivatives of the complete polypeptide, the fragment variant, or derivative may first be designed, and is then codon-optimized individually. Alternatively, a full-length polypeptide sequence is codon-optimized for a given species resulting in a codon-optimized coding region encoding the entire polypeptide, and then nucleic acid fragments of the codon-optimized coding region, which encode fragments, variants, and derivatives of the polypeptide are made from the original codon-optimized coding region. As would be well understood by those of ordinary skill in the art, if codons have been randomly assigned to the full-length coding region based on their frequency of use in a given species, nucleic acid fragments encoding fragments, variants, and derivatives would not necessarily be fully codon-optimized for the given species. However, such sequences are still much closer to the codon usage of the desired species than the native codon usage. The advantage of this approach is that synthesizing codon-optimized nucleic acid fragments encoding each fragment, variant, and derivative of a given polypeptide, although routine, would be time consuming and would result in significant expense.

When using the “full-optimization” method, the term “about” is used precisely to account for fractional percentages of codon frequencies for a given amino acid. As used herein, “about” is defined as one amino acid more or one amino acid less than the value given. The whole number value of amino acids is rounded up if the fractional frequency of usage is 0.50 or greater, and is rounded down if the fractional frequency of use is 0.49 or less. Using again the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 TUG codons, 12.87 percent of 62 equals 7.98 CUU codons, or “about 8,” i.e., 7, 8, or 9 CTU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

In a third method termed “minimal optimization,” coding regions are only partially optimized. For example, the invention includes a nucleic acid fragment of a codon-optimized coding region encoding a polypeptide in which at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence. Codons that are rarely found in the genes of the vertebrate of interest are changed to codons more commonly utilized in the coding regions of the vertebrate of interest.

This minimal human codon optimization for highly variant codons has several advantages, which include but are not limited to the following examples. Because fewer changes are made to the nucleotide sequence of the gene of interest, fewer manipulations are required, which leads to reduced risk of introducing unwanted mutations and lower cost, as well as allowing the use of commercially available site-directed mutagenesis kits, and reducing the need for expensive oligonucleotide synthesis. Further, decreasing the number of changes in the nucleotide sequence decreases the potential of altering the secondary structure of the sequence, which can have a significant impact on gene expression in certain host cells. The introduction of undesirable restriction sites is also reduced, facilitating the subcloning of the genes of interest into the plasmid expression vector.

The present invention also provides isolated polynucleotides comprising coding regions of measles virus polypeptides, e.g., HA, or F or fragments, variants, or derivatives thereof. The isolated polynucleotides can also be codon-optimized.

A human codon-optimized coding region which encodes SEQ ID NO:1 or 2 can be designed by any of the methods discussed herein. For “uniform” optimization, each amino acid is assigned the most frequent codon used in the human genome for that amino acid.

As described above, the term “about” means that the number of amino acids encoded by a certain codon may be one more or one less than the number given. It would be understood by those of ordinary skill in the art that the total number of any amino acid in the polypeptide sequence must remain constant, therefore, if there is one “more” of one codon encoding a given amino acid, there would have to be one “less” of another codon encoding that same amino acid.

In another form of minimal optimization, a Codon Usage Table (CUT) for the specific measles virus sequence in question is generated and compared to CUT for human genomic DNA. Amino acids are identified for which there is a difference of at least 10 percentage points in codon usage between human and measles virus DNA (either more or less). Then the measles virus codon is modified to conform to predominant human codon for each such amino acid. Furthermore, the remainder of codons for that amino acid are also modified such that they conform to the predominant human codon for each such amino acid.

### Compositions and Methods

In certain embodiments, the present invention is directed to compositions and methods of enhancing the immune response of a vertebrate in need of protection against measles virus infection by administering in vivo, into a tissue of a vertebrate, one or more polynucleotides comprising at least one codon-optimized coding region encoding a measles virus polypeptide, or a fragment, variant, or derivative thereof. In addition, the present invention is directed to compositions and methods of enhancing the immune response of a vertebrate in need of protection against measles virus infection by administering to the vertebrate a composition comprising one or more polynucleotides as described herein, and at least one isolated measles virus polypeptide, or a fragment, variant, or derivative thereof. The polynucleotide may be administered either prior to, at the same time (simultaneously), or subsequent to the administration of the isolated polypeptide.

The coding regions encoding measles virus polypeptides or fragments, variants, or derivatives thereof may be codon-optimized for a particular vertebrate. Codon optimization is carried out by the methods described herein, for example, in certain embodiments codon-optimized coding regions encoding polypeptides of measles virus, or nucleic acid fragments of such coding regions encoding fragments, variants, or derivatives thereof are optimized according to the codon usage of the particular vertebrate. The polynucleotides of the invention are incorporated into the cells of the vertebrate in vivo, and an immunologically effective amount of a measles virus polypeptide or a fragment, variant, or derivative thereof is produced in vivo. The coding regions encoding a measles virus polypeptide or a fragment, variant, or derivative thereof may be codon optimized for mammals, e.g., humans, apes, monkeys (e.g., owl, squirrel, cebus, rhesus, African green, patas, cynomolgus, and cercopithecus), orangutans, baboons, gibbons, and chimpanzees, dogs, wolves, cats, lions, and tigers, horses, donkeys, zebras, cows, pigs, sheep, deer, giraffes, bears, rabbits, mice, ferrets, seals, whales; birds, e.g., ducks, geese, terns, shearwaters, gulls, turkeys, chickens, quail, pheasants, geese, starlings and budgerigars, or other vertebrates.

In one embodiment, the present invention relates to codon-optimized coding regions encoding polypeptides of measles virus, or nucleic acid fragments of such coding regions fragments, variants, or derivatives thereof which have been optimized according to human codon usage. For example, human codon-optimized coding regions encoding polypeptides of measles virus, or fragments, variants, or derivatives thereof are prepared by substituting one or more codons preferred for use in human genes for the codons naturally used in the DNA sequence encoding the measles virus polypeptide or a fragment, variant, or derivative thereof. Also provided are polynucleotides, vectors, and other expression constructs comprising codon-optimized coding regions encoding polypeptides of measles virus, or nucleic acid fragments of such coding regions encoding fragments, variants, or derivatives thereof, pharmaceutical compositions comprising polynucleotides, vectors, and other expression constructs comprising codon-optimized regions encoding polypeptides of measles virus, or nucleic acid fragments of such coding regions encoding fragments, variants, or derivatives thereof, and various methods of using such polynucleotides, vectors and other expression constructs. Coding regions encoding measles virus polypeptides can be uniformly optimized, fully optimized, minimally optimized, codon-optimized by region and/or not codon-optimized, as described herein.

The present invention is further directed towards polynucleotides comprising codon-optimized coding regions encoding polypeptides of measles virus antigens, for example, HA, or F optionally in conjunction with other antigens. The invention is also directed to polynucleotides comprising codon-optimized nucleic acid fragments encoding fragments, variants and derivatives of these polypeptides.

In certain embodiments, the present invention provides an isolated polynucleotide comprising a nucleic acid fragment, where the nucleic acid fragment is a fragment of a codon-optimized coding region encoding a polypeptide at least 60%, 65%, 70%, 75%, 80%, 85%, 86%, 87%, 88%, 89%, 90%, 91%, 92%, 93%, 94%, 95%, 96%, 97%, 98%, or 99% identical to a measles virus polypeptide, e.g., HA, or F, and where the nucleic acid fragment is a variant of a codon-optimized coding region encoding a measles virus polypeptide, e.g., HA, or F. The human codon-optimized coding region can be optimized for any vertebrate species and by any of the methods described herein.

### Isolated Measles Virus Polypeptides

The present invention is further drawn to compositions which include at least one polynucleotide comprising one or more nucleic acid fragments, where each nucleic acid fragment is optionally a fragment of a codon-optimized coding region operably encoding a measles virus polypeptide or fragment, variant, or derivative thereof; together with one or more isolated measles virus component or isolated polypeptide. The measles virus component may be inactivated virus, attenuated virus, a viral vector expressing an isolated measles virus polypeptide, or a measles virus protein, fragment, variant or derivative thereof.

The polypeptides or fragments, variants or derivatives thereof, in combination with the codon-optimized nucleic acid compositions may be referred to as “combinatorial polynucleotide vaccine compositions” or “single formulation heterologous prime-boost vaccine compositions.”

The isolated measles virus polypeptides of the invention may be in any form, and are generated using techniques well known in the art. Examples include isolated measles virus proteins produced recombinantly, isolated measles virus proteins directly purified from their natural milieu, recombinant (non-measles virus) virus vectors expressing an isolated measles virus protein, or proteins delivered in the form of an inactivated measles virus vaccine, such as conventional vaccines.

In the instant invention, the combination of conventional antigen vaccine compositions with the codon-optimized nucleic acid compositions provides for therapeutically beneficial effects at dose sparing concentrations. For example, immunological responses sufficient for a therapeutically beneficial effect in patients predetermined for an approved commercial product, such as for the conventional product described above, can be attained by using less of the approved commercial product when supplemented or enhanced with the appropriate amount of codon-optimized nucleic acid. Thus, dose sparing is contemplated by administration of conventional measles virus vaccines administered in combination with the codon-optimized nucleic acids of the invention.

In particular, the dose of conventional vaccine may be reduced by at least 5%, at least 10%, at least 20%, at least 30%, at least 40%, at least 50%, at least 60% or at least 70% when administered in combination with the codon-optimized nucleic acid compositions of the invention.

Similarly, a desirable level of an immunological response afforded by a DNA-based pharmaceutical alone may be attained with less DNA by including an aliquot of a conventional vaccine. Further, using a combination of conventional and DNA-based pharmaceuticals may allow both materials to be used in lesser amounts while still affording the desired level of immune response arising from administration of either component alone in higher amounts (e.g., one may use less of either immunological product when they are used in combination). This may be manifest not only by using lower amounts of materials being delivered at any time, but also to reducing the number of administrations points in a vaccination regime (e.g., 2 versus 3 or 4 injections), and/or to reducing the kinetics of the immunological response (e.g., desired response levels are attained in 3 weeks instead of 6 after immunization).

In particular, the dose of DNA based pharmaceuticals, may be reduced by at least 5%, at least 10%, at least 20%, at least 30%, at least 40%, at least 50%, at least 60% or at least 70% when administered in combination with conventional measles virus vaccines.

Determining the precise amounts of DNA-based pharmaceutical and conventional antigen is based on a number of factors as described above, and is readily determined by one of ordinary skill in the art.

In addition to dose sparing, the claimed combinatorial compositions provide for a broadening of the immune response and/or enhanced beneficial immune responses. Such broadened or enhanced immune responses are achieved by: adding DNA to enhance cellular responses to a conventional vaccine; adding a conventional vaccine to a DNA pharmaceutical to enhance humoral response; using a combination that induces additional epitopes (both humoral and/or cellular) to be recognized and/or more desirably responded to (epitope broadening); employing a DNA-conventional vaccine combination designed for a particular desired spectrum of immunological responses; obtaining a desirable spectrum by using higher amounts of either component. The broadened immune response is measurable by one of ordinary skill in the art by standard immunological assay specific for the desirable response spectrum.

Both broadening and dose sparing can be obtained simultaneously.

The isolated measles virus polypeptide or fragment, variant, or derivative thereof to be delivered (either a recombinant protein, a purified subunit, or viral vector expressing an isolated measles virus polypeptide, or in the form of an inactivated measles virus vaccine) can be any isolated measles virus polypeptide or fragment, variant, or derivative thereof, including but not limited to the HA, or F proteins or fragments, variants or derivatives thereof. It should be noted that any isolated measles virus polypeptide or fragment, variant, or derivative thereof described herein can be combined in a composition with any polynucleotide comprising a nucleic acid fragment, where the nucleic acid fragment is optionally a fragment of a codon-optimized coding region operably encoding a measles virus polypeptide or fragment, variant, or derivative thereof. The proteins can be different, the same, or can be combined in any combination of one or more isolated measles virus proteins and one or more polynucleotides.

In certain embodiments, the isolated measles virus polypeptides, or fragments, derivatives or variants thereof can be fused to or conjugated to a second isolated measles virus polypeptide, or fragment, derivative or variant thereof, or can be fused to other heterologous proteins, including for example, hepatitis B proteins including, but not limited to the hepatitis B core antigen (HBcAg), or those derived from diphtheria or tetanus. The second isolated measles virus polypeptide or other heterologous protein can act as a “carrier” that potentiates the immunogenicity of the measles virus polypeptide or a fragment, variant, or derivative thereof to which it is attached. Hepatitis B virus proteins and fragments and variants thereof useful as carriers within the scope of the invention are disclosed in U.S. Pat. Nos. 6,231,864 and 5,143,726. Polynucleotides comprising coding regions encoding said fused or conjugated proteins are also within the scope of the invention.

The use of recombinant particles comprising hepatitis B core antigen (“HBcAg”) and heterologous protein sequences as potent immunogenic moieties is well documented. For example, addition of heterologous sequences to the amino terminus of a recombinant HBcAg results in the spontaneous assembly of particulate structures which express the heterologous epitope on their surface, and which are highly immunogenic when inoculated into experimental animals. See Clarke et al., Nature 330:381-384 (1987). Heterologous epitopes can also be inserted into HBcAg particles by replacing approximately 40 amino acids of the carboxy terminus of the protein with the heterologous sequences. These recombinant HBcAg proteins also spontaneously form immunogenic particles. See Stahl and Murray, Proc. Natl. Acad. Sci. USA, 86:6283-6287 (1989). Additionally, chimeric HBcAg particles may be constructed where the heterologous epitope is inserted in or replaces all or part of the sequence of amino acid residues in a more central region of the HBcAg protein, in an immunodominant loop, thereby allowing the heterologous epitope to be displayed on the surface of the resulting particles. See EP Patent No. 0421635 B1 and Galibert, F., et al., Nature 281:646-650 (1979); see also U.S. Pat. Nos. 4,818,527, 4,882,145 and 5,143,726.

Chimeric HBcAg particles comprising isolated measles virus proteins or variants, fragments or derivatives thereof are prepared by recombinant techniques well known to those of ordinary skill in the art. A polynucleotide, e.g., a plasmid, which carries the coding region for the HBcAg operably associated with a promoter is constructed. Convenient restriction sites are engineered into the coding region encoding the N-terminal, central, and/or C-terminal portions of the HBcAg, such that heterologous sequences may be inserted. A construct which expresses an HBcAg/measles virus fusion protein is prepared by inserting a DNA sequence encoding a measles virus protein or variant, fragment or derivative thereof, in frame, into a desired restriction site in the coding region of the HBcAg. The resulting construct is then inserted into a suitable host cell, e.g., E. coli, under conditions where the chimeric HBcAg will be expressed. The chimeric HBcAg self-assembles into particles when expressed, and can then be isolated, e.g., by ultracentrifugation. The particles formed resemble the natural 27 nm HBcAg particles isolated from a hepatitis B virus, except that an isolated measles virus protein or fragment, variant, or derivative thereof is contained in the particle, preferably exposed on the outer particle surface.

The measles virus protein or fragment, variant, or derivative thereof expressed in a chimeric HBcAg particle may be of any size which allows suitable particles of the chimeric HBcAg to self-assemble. As discussed above, even small antigenic epitopes may be immunogenic when expressed in the context of an immunogenic carrier, e.g., a HBcAg. Thus, HBcAg particles of the invention may comprise at least 4, at least 5, at least 6, at least 7, at least 8, at least 9, at least 10, at least 15, at least 20, at least 25, or between about 15 to about 30 amino acids of a measles virus protein fragment of interest inserted therein. HBcAg particles of the invention may further comprise immunogenic or antigenic epitopes of at least 5, 10, 15, 20, 25, 30, 35, 40, 45, 50, 55, 60, 65, 70, 75, 80, 85, 90, 95, or 100 amino acid residues of a measles virus protein fragment of interest inserted therein.

The immunodominant loop region of HBcAg was mapped to about amino acid residues 75 to 83, to about amino acids 75 to 85 or to about amino acids 130 to 140. See Colucci et al., J. Immunol. 141:4376-4380 (1988), and Salfeld et al., J. Virol. 63:798 (1989). A chimeric HBcAg is still often able to form core particles when foreign epitopes are cloned into the immunodominant loop. Thus, for example, amino acids of the measles virus protein fragment may be inserted into the sequence of HBcAg amino acids at various positions, for example, at the N-terminus, from about amino acid 75 to about amino acid 85, from about amino acid 75 to about amino acid 83, from about amino acid 130 to about amino acid 140, or at the C-terminus. Where amino acids of the measles virus protein fragment replace all or part of the native core protein sequence, the inserted measles virus sequence is generally not shorter, but may be longer, than the HBcAg sequence it replaces.

Alternatively, if particle formation is not desired, full-length measles virus coding sequences can be fused to the coding region for the HBcAg. The HBcAg sequences can be fused either at the N- or C-terminus of any of the Measles antigens described herein. Fusions could include flexible protein linkers. These fusion constructs could be codon optimized by any of the methods described.

The chimeric HBcAg can be used in the present invention in conjunction with a polynucleotide comprising a nucleic acid fragment, where each nucleic acid fragment is optionally a fragment of a codon-optimized coding region operably encoding a measles virus polypeptide, or a fragment, variant, or derivative thereof, as a measles vaccine for a vertebrate.

### Methods and Administration

The present invention also provides methods for delivering a measles virus polypeptide or a fragment, variant, or derivative thereof to a human, which comprise administering to a human one or more of the compositions described herein; such that upon administration of compositions such as those described herein, a measles virus polypeptide or a fragment, variant, or derivative thereof is expressed in human cells, in an amount sufficient to generate an immune response to the measles virus or administering the measles virus polypeptide or a fragment, variant, or derivative thereof itself to the human in an amount sufficient to generate an immune response.

The present invention further provides methods for delivering a measles virus polypeptide or a fragment, variant, or derivative thereof to a human, which comprise administering to a vertebrate one or more of the compositions described herein; such that upon administration of compositions such as those described herein, an immune response is generated in the vertebrate.

The term “vertebrate” is intended to encompass a singular “vertebrate” as well as plural “vertebrates” and comprises mammals and birds, as well as fish, reptiles, and amphibians.

The term “mammal” is intended to encompass a singular “mammal” and plural “mammals,” and includes, but is not limited to humans; primates such as apes, monkeys (e.g., owl, squirrel, cebus, rhesus, African green, patas, cynomolgus, and cercopithecus), orangutans, baboons, gibbons, and chimpanzees; canids such as dogs and wolves; felids such as cats, lions, and tigers; equines such as horses, donkeys, and zebras, food animals such as cows, pigs, and sheep; ungulates such as deer and giraffes; ursids such as bears; and others such as rabbits, mice, ferrets, seals, whales. In particular, the mammal can be a human subject, a food animal or a companion animal.

The term “bird” is intended to encompass a singular “bird” and plural “birds,” and includes, but is not limited to, feral water birds such as ducks, geese, terns, shearwaters, and gulls; as well as domestic avian species such as turkeys, chickens, quail, pheasants, geese, and ducks. The term “bird” also encompasses passerine birds such as starlings and budgerigars.

The present invention further provides a method for generating, enhancing or modulating an immune response to a measles virus comprising administering to a vertebrate one or more of the compositions described herein. In this method, the compositions may include one or more isolated polynucleotides comprising at least one nucleic acid fragment where the nucleic acid fragment is optionally a fragment of a codon-optimized coding region encoding a measles virus polypeptide, or a fragment, variant, or derivative thereof. In another embodiment, the compositions may include both a polynucleotide as described above, and also an isolated measles virus polypeptide, or a fragment, variant, or derivative thereof, wherein the protein is provided as a recombinant protein, in particular, a fusion protein, a purified subunit, viral vector expressing the protein, or in the form of an inactivated measles virus vaccine. Thus, the latter compositions include both a polynucleotide encoding a measles virus polypeptide or a fragment, variant, or derivative thereof and an isolated measles virus polypeptide or a fragment, variant, or derivative thereof. The measles virus polypeptide or a fragment, variant, or derivative thereof encoded by the polynucleotide of the compositions need not be the same as the isolated measles virus polypeptide or a fragment, variant, or derivative thereof of the compositions. Compositions to be used according to this method may be univalent, bivalent, trivalent or multivalent.

The polynucleotides of the compositions may comprise a fragment of a human (or other vertebrate) codon-optimized coding region encoding a protein of the measles virus, or a fragment, variant, or derivative thereof. The polynucleotides are incorporated into the cells of the vertebrate in vivo, and an antigenic amount of the measles virus polypeptide, or fragment, variant, or derivative thereof, is produced in vivo. Upon administration of the composition according to this method, the measles virus polypeptide or a fragment, variant, or derivative thereof is expressed in the vertebrate in an amount sufficient to elicit an immune response. Such an immune response might be used, for example, to generate antibodies to the measles virus for use in diagnostic assays or as laboratory reagents, or as therapeutic or preventative vaccines as described herein.

The present invention further provides a method for generating, enhancing, or modulating a protective and/or therapeutic immune response to measles virus in a vertebrate, comprising administering to a vertebrate in need of therapeutic and/or preventative immunity one or more of the compositions described herein. In this method, the compositions include one or more polynucleotides comprising at least one nucleic acid fragment, where the nucleic acid fragment is optionally a fragment of a codon-optimized coding region encoding a measles virus polypeptide, or a fragment, variant, or derivative thereof. In a further embodiment, the composition used in this method includes both an isolated polynucleotide comprising at least one nucleic acid fragment, where the nucleic acid fragment is optionally a fragment of a codon-optimized coding region encoding a measles virus polypeptide, or a fragment, variant, or derivative thereof; and at least one isolated measles virus polypeptide, or a fragment, variant, or derivative thereof. Thus, the latter composition includes both an isolated polynucleotide encoding a measles virus polypeptide or a fragment, variant, or derivative thereof and an isolated measles virus polypeptide or a fragment, variant, or derivative thereof, for example, a recombinant protein, a purified subunit, viral vector expressing the protein, or an inactivated virus vaccine. Upon administration of the composition according to this method, the measles virus polypeptide or a fragment, variant, or derivative thereof is expressed in the human in a therapeutically or prophylactically effective amount.

As used herein, an “immune response” refers to the ability of a vertebrate to elicit an immune reaction to a composition delivered to that vertebrate. Examples of immune responses include an antibody response or a cellular, e.g., cytotoxic T-cell, response. One or more compositions of the present invention may be used to prevent measles infection in vertebrates, e.g., as a prophylactic vaccine, to establish or enhance immunity to measles virus in a healthy individual prior to exposure to measles or contraction of measles disease, thus preventing the disease or reducing the severity of disease symptoms.

As mentioned above, compositions of the present invention can be used both to prevent measles virus infection, and also to therapeutically treat measles virus infection. In individuals already exposed to measles, or already suffering from measles disease, the present invention is used to further stimulate the immune system of the vertebrate, thus reducing or eliminating the symptoms associated with that disease or disorder. As defined herein, “treatment” refers to the use of one or more compositions of the present invention to prevent, cure, retard, or reduce the severity of measles disease symptoms in a vertebrate, and/or result in no worsening of measles disease over a specified period of time in a vertebrate which has already been exposed to measles virus and is thus in need of therapy. The term “prevention” refers to the use of one or more compositions of the present invention to generate immunity in a vertebrate which has not yet been exposed to a particular strain of measles virus, thereby preventing or reducing disease symptoms if the vertebrate is later exposed to the particular strain of measles virus. The methods of the present invention therefore may be referred to as therapeutic vaccination or preventative or prophylactic vaccination. It is not required that any composition of the present invention provide total immunity to measles or totally cure or eliminate all measles disease symptoms. As used herein, a “vertebrate in need of therapeutic and/or preventative immunity” refers to an individual for whom it is desirable to treat, i.e., to prevent, cure, retard, or reduce the severity of measles disease symptoms, and/or result in no worsening of measles disease over a specified period of time.

One or more compositions of the present invention are utilized in a “prime boost” regimen. An example of a “prime boost” regimen may be found in Yang, Z. et al., J. Virol. 77:799-803 (2002). In these embodiments, one or more polynucleotide vaccine compositions of the present invention are delivered to a vertebrate, thereby priming the immune response of the vertebrate to a measles virus, and then a second immunogenic composition is utilized as a boost vaccination. One or more compositions of the present invention are used to prime immunity, and then a second immunogenic composition, e.g., a recombinant viral vaccine or vaccines, a different polynucleotide vaccine, or one or more purified subunit isolated measles virus polypeptides or fragments, variants or derivatives thereof is used to boost the anti-measles virus immune response.

In one embodiment, a priming composition and a boosting composition are combined in a single composition or single formulation. For example, a single composition may comprise an isolated measles virus polypeptide or a fragment, variant, or derivative thereof as the priming component and a polynucleotide encoding a measles protein as the boosting component. In this embodiment, the compositions may be contained in a single vial where the priming component and boosting component are mixed together. In general, because the peak levels of expression of protein from the polynucleotide does not occur until later (e.g., 7-10 days) after administration, the polynucleotide component may provide a boost to the isolated protein component. Compositions comprising both a priming component and a boosting component are referred to herein as “combinatorial vaccine compositions” or “single formulation heterologous prime-boost vaccine compositions.” In addition, the priming composition may be administered before the boosting composition, or even after the boosting composition, if the boosting composition is expected to take longer to act.

In another embodiment, the priming composition may be administered simultaneously with the boosting composition, but in separate formulations where the priming component and the boosting component are separated.

The terms “priming” or “primary” and “boost” or “boosting” as used herein may refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously.

In certain embodiments, one or more compositions of the present invention are delivered to a vertebrate by methods described herein, thereby achieving an effective therapeutic and/or an effective preventative immune response. More specifically, the compositions of the present invention may be administered to any tissue of a vertebrate, including, but not limited to, muscle, skin, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, and connective tissue, e.g., cartilage.

Furthermore, the compositions of the present invention may be administered to any internal cavity of a vertebrate, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland or a liver. When the compositions of the present invention are administered to the lumen of a duct of a salivary gland or liver, the desired polypeptide is expressed in the salivary gland and the liver such that the polypeptide is delivered into the blood stream of the vertebrate from each of the salivary gland or the liver. Certain modes for administration to secretory organs of a gastrointestinal system using the salivary gland, liver and pancreas to release a desired polypeptide into the bloodstream is disclosed in U.S. Pat. Nos. 5,837,693 and 6,004,944, both of which are incorporated herein by reference in their entireties.

In certain embodiments, the compositions are administered to muscle, either skeletal muscle or cardiac muscle, or to lung tissue. Specific, but non-limiting modes for administration to lung tissue are disclosed in Wheeler, C. J., et al., Proc. Natl. Acad. Sci. USA 93:11454-11459 (1996), which is incorporated herein by reference in its entirety.

According to the disclosed methods, compositions of the present invention can be administered by intramuscular (i.m.), interdermal (i.d.), subcutaneous (s.c.), or intrapulmonary routes. Other suitable routes of administration include, but are not limited to intratracheal, transdermal, intraocular, intranasal, inhalation, intracavity, intravenous (i.v.), intraductal (e.g., into the pancreas) and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), inraatrial (i.e., into the heart atrium) and sub arachnoid (i.e., into the sub arachnoid spaces of the brain) administration.

For oral indications, the present invention may be administered in the form of tongue strips wherein the composition is embedded or applied to the strip. The user places the strip on the tongue and the strip melts or dissolves in the mouth thereby releasing the composition.

Any mode of administration can be used so long as the mode results in the expression of the desired peptide or protein, in the desired tissue, in an amount sufficient to generate an immune response to measles virus and/or to generate a prophylactically or therapeutically effective immune response to measles virus in a human in need of such response. Administration means of the present invention include needle injection, catheter infusion, biolistic injectors, particle accelerators (e.g., “gene guns” or pneumatic “needleless” injectors) Med-E-Jet (Vahlsing, H., et al., J. Immunol. Methods 171:11-22 (1994)), Pigjet (Schrijver, R., et al., Vaccine 15: 1908-1916 (1997)), Biojector (Davis, H., et al., Vaccine 12: 1503-1509 (1994); Gramzinski, R., et al., Mol. Med. 4: 109-118 (1998)), AdvantaJet (Linmayer, I., et al., Diabetes Care 9:294-297 (1986)), Medi-jector (Martins, J., and Roedl, E. J. Occup. Med. 21:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65: 2193-2203 (1999)) or topical applications during surgery. Certain modes of administration are intramuscular needle-based injection and pulmonary application via catheter infusion. Energy-assisted plasmid delivery (EAPD) methods may also be employed to administer the compositions of the invention. One such method involves the application of brief electrical pulses to injected tissues, a procedure commonly known as electroporation. See generally Mir, L. M. et al., Proc. Natl. Acad. Sci. USA 96:4262-7 (1999); Hartikka, J. et al., Mol. Ther. 4:407-15 (2001); Mathiesen, I., Gene Ther. 6:508-14 (1999); Rizzuto G. et al., Hum. Gen. Ther. 11:1891-900 (2000).

Determining an effective amount of one or more compositions of the present invention depends upon a number of factors including, for example, the antigen being expressed or administered directly, e.g., HA, or F, or fragments, variants, or derivatives thereof, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian.

Compositions of the present invention may include various salts, excipients, delivery vehicles and/or auxiliary agents as are disclosed, e.g., in U.S. patent application Publication No. 2002/0019358, published Feb. 14, 2002.

Furthermore, compositions of the present invention may include one or more transfection facilitating compounds that facilitate delivery of polynucleotides to the interior of a cell, and/or to a desired location within a cell. As used herein, the terms “transfection facilitating compound,” “transfection facilitating agent,” and “transfection facilitating material” are synonymous, and may be used interchangeably. It should be noted that certain transfection facilitating compounds may also be “adjuvants” as described infra, i.e., in addition to facilitating delivery of polynucleotides to the interior of a cell, the compound acts to alter or increase the immune response to the antigen encoded by that polynucleotide. Examples of the transfection facilitating compounds include, but are not limited to, inorganic materials such as calcium phosphate, alum (aluminum sulfate), and gold particles (e.g., “powder” type delivery vehicles); peptides that are, for example, cationic, intercell targeting (for selective delivery to certain cell types), intracell targeting (for nuclear localization or endosomal escape), and ampipathic (helix forming or pore forming); proteins that are, for example, basic (e.g., positively charged) such as histones, targeting (e.g., asialoprotein), viral (e.g., Sendai virus coat protein), and pore-forming; lipids that are, for example, cationic (e.g., DMRIE, DOSPA, DC-Chol), basic (e.g., steryl amine), neutral (e.g., cholesterol), anionic (e.g., phosphatidyl serine), and zwitterionic (e.g., DOPE, DOPC); and polymers such as dendrimers, star-polymers, “homogenous” poly-amino acids (e.g., poly-lysine, poly-arginine), “heterogeneous” poly-amino acids (e.g., mixtures of lysine & glycine), co-polymers, polyvinylpyrrolidinone (PVP), poloxamers (e.g. CRL 1005) and polyethylene glycol (PEG). A transfection facilitating material can be used alone or in combination with one or more other transfection facilitating materials. Two or more transfection facilitating materials can be combined by chemical bonding (e.g., covalent and ionic such as in lipidated polylysine, PEGylated polylysine) (Toncheva, et al., Biochim. Biophys. Acta 1380(3):354-368 (1988)), mechanical mixing (e.g., free moving materials in liquid or solid phase such as “polylysine+cationic lipids”) (Gao and Huang, Biochemistry 35:1027-1036 (1996); Trubetskoy, et al., Biochem. Biophys. Acta 1131:311-313 (1992)), and aggregation (e.g., co-precipitation, gel forming such as in cationic lipids+poly-lactide, and polylysine+gelatin).

One category of transfection facilitating materials is cationic lipids. Examples of cationic lipids are 5-carboxyspermylglycine dioctadecylamide (DOGS) and dipalmitoyl-phophatidylethanolamine-5-carboxyspermylamide (DPPES). Cationic cholesterol derivatives are also useful, including {3β-[N—N′,N′-dimethylamino)ethane]-carbomoyl}-cholesterol (DC-Chol). Dimethyldioctdecyl-ammonium bromide (DDAB), N-(3-aminopropyl)-N,N-(bis-(2-tetradecyloxyethyl))-N-methyl-ammonium bromide (PA-DEMO), N-(3-aminopropyl)-N,N-(bis-(2-dodecyloxyethyl))-N-methyl-ammonium bromide (PA-DELO), N,N,N-tris-(2-dodecyloxy)ethyl-N-(3-amino)propyl-ammonium bromide (PA-TELO), and N1-(3-aminopropyl)((2-dodecyloxy)ethyl)-N2-(2-dodecyloxy)ethyl-1-piperazinaminium bromide (GA-LOE-BP) can also be employed in the present invention.

Non-diether cationic lipids, such as DL-1,2-dioleoyl-3-dimethylaminopropyl-β-hydroxyethylammonium (DORI diester), 1-O-oleyl-2-oleoyl-3-dimethylaminopropyl-p-hydroxyethylammonium (DORI ester/ether), and their salts promote in vivo gene delivery. In some embodiments, cationic lipids comprise groups attached via a heteroatom attached to the quaternary ammonium moiety in the head group. A glycyl spacer can connect the linker to the hydroxyl group.

Specific, but non-limiting cationic lipids for use in certain embodiments of the present invention include DMRIE ((±)-N-(2-hydroxyethyl)-N,N-dimethyl-2,3-bis(tetradecyloxy)-1-propanam-inium bromide), GAP-DMORIE ((±)—N-(3-aminopropyl)-N,N-dimethyl-2,3-bis(syn-9-tetradeceneyloxy)-1-propanaminium bromide), and GAP-DMRIE ((±)—N-(3-aminopropyl)-N,N-dimethyl-2,3-(bis-dodecyloxy)-1-propaniminium bromide).

Other specific but non-limiting cationic surfactants for use in certain embodiments of the present invention include Bn-DHRIE, DhxRIE, DhxRIE-OAc, DhxRIE-OBz and Pr-DOctRIE-OAc. These lipids are disclosed in copending U.S. patent application Ser. No. 10/725,015. In another aspect of the present invention, the cationic surfactant is Pr-DOctRIE-OAc.

Other cationic lipids include (±)-N,N-dimethyl-N-[2-(sperminecarboxamido)ethyl]-2,3-bis(dioleyloxy)-1-propaniminium pentahydrochloride (DOSPA), (±)-N-(2-aminoethyl)-N,N-dimethyl-2,3-bis(tetradecyloxy)-1-propaniminium bromide (β-aminoethyl-DMRIE or βAE-DMRIE) (Wheeler, et al., Biochim. Biophys. Acta 1280:1-11 (1996), and (±)-N-(3-aminopropyl)-N,N-dimethyl-2,3-bis(dodecyloxy)-1-propaniminium bromide (GAP-DLRIE) (Wheeler, et al., Proc. Natl. Acad. Sci. USA 93:11454-11459 (1996)), which have been developed from DMRIE.

Other examples of DMRIE-derived cationic lipids that are useful for the present invention are (±)-N-(3-aminopropyl)-N,N-dimethyl-2,3-(bis-decyloxy)-1-propanaminium bromide (GAP-DDRIE), (±)-N-(3-aminopropyl)-N,N-dimethyl-2,3-(bis-tetradecyloxy)-1-propanami-nium bromide (GAP-DMRIE), (±)-N-((N″-methyl)-N′-ureyl)propyl-N,N-dimethyl-2,3-bis(tetradecyloxy-)-1-propanaminium bromide (GMU-DMRIE), (±)-N-(2-hydroxyethyl)-N,N-dimethyl-2,3-bis(dodecyloxy)-1-propanaminium bromide (DLRIE), and (±)—N-(2-hydroxyethyl)-N,N-dimethyl-2,3-bis-([Z]-9-octadecenyloxy)propyl-1-propaniminium bromide (HP-DORIE).

In the embodiments where the immunogenic composition comprises a cationic lipid, the cationic lipid may be mixed with one or more co-lipids. For purposes of definition, the term “co-lipid” refers to any hydrophobic material which may be combined with the cationic lipid component and includes amphipathic lipids, such as phospholipids, and neutral lipids, such as cholesterol. Cationic lipids and co-lipids may be mixed or combined in a number of ways to produce a variety of non-covalently bonded macroscopic structures, including, for example, liposomes, multilamellar vesicles, unilamellar vesicles, micelles, and simple films. One non-limiting class of co-lipids are the zwitterionic phospholipids, which include the phosphatidylethanolamines and the phosphatidylcholines. Examples of phosphatidylethanolamines, include DOPE, DMPE and DPyPE. In certain embodiments, the co-lipid is DPyPE which comprises two phytanoyl substituents incorporated into the diacylphosphatidylethanolamine skeleton and the cationinc lipid is GAP-DMORIE, (resulting in Vaxfectin® adjuvant). In other embodiments, the co-lipid is DOPE, the CAS name is 1,2-diolyeoyl-sn-glycero-3-phosphoethanolamine.

When a composition of the present invention comprises a cationic lipid and co-lipid, the cationic lipid:co-lipid molar ratio may be from about 9:1 to about 1:9, from about 4:1 to about 1:4, from about 2:1 to about 1:2, or about 1:1.

In order to maximize homogeneity, the cationic lipid and co-lipid components may be dissolved in a solvent such as chloroform, followed by evaporation of the cationic lipid/co-lipid solution under vacuum to dryness as a film on the inner surface of a glass vessel (e.g., a Rotovap round-bottomed flask). Upon suspension in an aqueous solvent, the amphipathic lipid component molecules self-assemble into homogenous lipid vesicles. These lipid vesicles may subsequently be processed to have a selected mean diameter of uniform size prior to complexing with, for example, a codon-optimized polynucleotide of the present invention, according to methods known to those skilled in the art. For example, the sonication of a lipid solution is described in Felgner et al., Proc. Natl. Acad. Sci. USA 8:7413-7417 (1987) and in U.S. Pat. No. 5,264,618.

In those embodiments where the composition includes a cationic lipid, polynucleotides of the present invention are complexed with lipids by mixing, for example, a plasmid in aqueous solution and a solution of cationic lipid:co-lipid as prepared herein are mixed. The concentration of each of the constituent solutions can be adjusted prior to mixing such that the desired final plasmid/cationic lipid:co-lipid ratio and the desired plasmid final concentration will be obtained upon mixing the two solutions. The cationic lipid:co-lipid mixtures are suitably prepared by hydrating a thin film of the mixed lipid materials in an appropriate volume of aqueous solvent by vortex mixing at ambient temperatures for about 1 minute. The thin films are prepared by admixing chloroform solutions of the individual components to afford a desired molar solute ratio followed by aliquoting the desired volume of the solutions into a suitable container. The solvent is removed by evaporation, first with a stream of dry, inert gas (e.g., argon) followed by high vacuum treatment.

Other hydrophobic and amphiphilic additives, such as, for example, sterols, fatty acids, gangliosides, glycolipids, lipopeptides, liposaccharides, neobees, niosomes, prostaglandins and sphingolipids, may also be included in compositions of the present invention. In such compositions, these additives may be included in an amount between about 0.1 mol % and about 99.9 mol % (relative to total lipid), about 1-50 mol %, or about 2-25 mol %.

Additional embodiments of the present invention are drawn to compositions comprising an auxiliary agent which is administered before, after, or concurrently with the polynucleotide. As used herein, an “auxiliary agent” is a substance included in a composition for its ability to enhance, relative to a composition which is identical except for the inclusion of the auxiliary agent, the entry of polynucleotides into vertebrate cells in vivo, and/or the in vivo expression of polypeptides encoded by such polynucleotides. Certain auxiliary agents may, in addition to enhancing entry of polynucleotides into cells, enhance an immune response to an immunogen encoded by the polynucleotide. Auxiliary agents of the present invention include nonionic, anionic, cationic, or zwitterionic surfactants or detergents, with nonionic surfactants or detergents being preferred, chelators, DNase inhibitors, poloxamers, agents that aggregate or condense nucleic acids, emulsifying or solubilizing agents, wetting agents, gel-forming agents, and buffers.

Auxiliary agents for use in compositions of the present invention include, but are not limited to non-ionic detergents and surfactants IGEPAL CA 6300, NONIDET NP-40, Nonidet® P40, Tween-20™, Tween-80™, Pluronic® F68 (ave. MW: 8400; approx. MW of hydrophobe, 1800; approx. wt. % of hydrophile, 80%), Pluronic F77® (ave. MW: 6600; approx. MW of hydrophobe, 2100; approx. wt. % of hydrophile, 70%), Pluronic P65® (ave. MW: 3400; approx. MW of hydrophobe, 1800; approx. wt. % of hydrophile, 50%), Triton X-100™, and Triton X-114™; the anionic detergent sodium dodecyl sulfate (SDS); the sugar stachyose; the condensing agent DMSO; and the chelator/DNAse inhibitor EDTA, CRL 1005 (12 kDa, 5% POE), and BAK (Benzalkonium chloride 50% solution, available from Ruger Chemical Co. Inc.). In certain specific embodiments, the auxiliary agent is DMSO, Nonidet P40, Pluronic F68® (ave. MW: 8400; approx. MW of hydrophobe, 1800; approx. wt. % of hydrophile, 80%), Pluronic F77® (ave. MW: 6600; approx. MW of hydrophobe, 2100; approx. wt. % of hydrophile, 70%), Pluronic P65® (ave. MW: 3400; approx. MW of hydrophobe, 1800; approx. wt. % of hydrophile, 50%), Pluronic L64® (ave. MW: 2900; approx. MW of hydrophobe, 1800; approx. wt. % of hydrophile, 40%), and Pluronic F108® (ave. MW: 14600; approx. MW of hydrophobe, 3000; approx. wt. % of hydrophile, 80%). See, e.g., U.S. patent application Publication No. 2002/0019358, published Feb. 14, 2002.

Certain compositions of the present invention can further include one or more adjuvants before, after, or concurrently with the polynucleotide. The term “adjuvant” refers to any material having the ability to (1) alter or increase the immune response to a particular antigen or (2) increase or aid an effect of a pharmacological agent. It should be noted, with respect to polynucleotide vaccines, that an “adjuvant,” can be a transfection facilitating material. Similarly, certain “transfection facilitating materials” described supra, may also be an “adjuvant.” An adjuvant may be used with a composition comprising a polynucleotide of the present invention. In a prime-boost regimen, as described herein, an adjuvant may be used with either the priming immunization, the booster immunization, or both. Suitable adjuvants include, but are not limited to, cytokines and growth factors; bacterial components (e.g., endotoxins, in particular superantigens, exotoxins and cell wall components); aluminum-based salts; calcium-based salts; silica; polynucleotides; toxoids; serum proteins, viruses and virally-derived materials, poisons, venoms, imidazoquiniline compounds, poloxamers, and cationic lipids.

A great variety of materials have been shown to have adjuvant activity through a variety of mechanisms. Any compound which may increase the expression, antigenicity or immunogenicity of the polypeptide is a potential adjuvant. The present invention provides an assay to screen for improved immune responses to potential adjuvants. Potential adjuvants which may be screened for their ability to enhance the immune response according to the present invention include, but are not limited to: inert carriers, such as alum, bentonite, latex, and acrylic particles; pluronic block polymers, such as TiterMax® (block copolymer CRL-8941, squalene (a metabolizable oil) and a microparticulate silica stabilizer); depot formers, such as Freunds adjuvant, surface active materials, such as saponin, lysolecithin, retinal, Quil A, liposomes, and pluronic polymer formulations; macrophage stimulators, such as bacterial lipopolysaccharide; alternate pathway complement activators, such as insulin, zymosan, endotoxin, and levamisole; and non-ionic surfactants, such as poloxamers, poly(oxyethylene)-poly(oxypropylene) tri-block copolymers. Also included as adjuvants are transfection-facilitating materials, such as those described above.

Poloxamers which may be screened for their ability to enhance the immune response according to the present invention include, but are not limited to, commercially available poloxamers such as Pluronic® surfactants, which are block copolymers of propylene oxide and ethylene oxide in which the propylene oxide block is sandwiched between two ethylene oxide blocks. Examples of Pluronic® surfactants include Pluronic® L121 (ave. MW: 4400; approx. MW of hydrophobe, 3600; approx. wt. % of hydrophile, 10%), Pluronic® L101 (ave. MW: 3800; approx. MW of hydrophobe, 3000; approx. wt. % of hydrophile, 10%), Pluronic® L81 (ave. MW: 2750; approx. MW of hydrophobe, 2400; approx. wt. % of hydrophile, 10%), Pluronic® L61 (ave. MW: 2000; approx. MW of hydrophobe, 1800; approx. wt. % of hydrophile, 10%), Pluronic® L31 (ave. MW: 1100; approx. MW of hydrophobe, 900; approx. wt. % of hydrophile, 10%), Pluronic® L122 (ave. MW: 5000; approx. MW of hydrophobe, 3600; approx. wt. % of hydrophile, 20%), Pluronic® L92 (ave. MW: 3650; approx. MW of hydrophobe, 2700; approx. wt. % of hydrophile, 20%), Pluronic® L72 (ave. MW: 2750; approx. MW of hydrophobe, 2100; approx. wt. % of hydrophile, 20%), Pluronic® L62 (ave. MW: 2500; approx. MW of hydrophobe, 1800; approx. wt. % of hydrophile, 20%), Pluronic® L42 (ave. MW: 1630; approx. MW of hydrophobe, 1200; approx. wt. % of hydrophile, 20%), Pluronic® L63 (ave. MW: 2650; approx. MW of hydrophobe, 1800; approx. wt. % of hydrophile, 30%), Pluronic® L43 (ave. MW: 1850; approx. MW of hydrophobe, 1200; approx. wt. % of hydrophile, 30%), Pluronic® L64 (ave. MW: 2900; approx. MW of hydrophobe, 1800; approx. wt. % of hydrophile, 40%), Pluronic® L44 (ave. MW: 2200; approx. MW of hydrophobe, 1200; approx. wt. % of hydrophile, 40%), Pluronic® L35 (ave. MW: 1900; approx. MW of hydrophobe, 900; approx. wt. % of hydrophile, 50%), Pluronic® P123 (ave. MW: 5750; approx. MW of hydrophobe, 3600; approx. wt. % of hydrophile, 30%), Pluronic® P103 (ave. MW: 4950; approx. MW of hydrophobe, 3000; approx. wt. % of hydrophile, 30%), Pluronic® P104 (ave. MW: 5900; approx. MW of hydrophobe, 3000; approx. wt. % of hydrophile, 40%), Pluronic® P84 (ave. MW: 4200; approx. MW of hydrophobe, 2400; approx. wt. % of hydrophile, 40%), Pluronic® P105 (ave. MW: 6500; approx. MW of hydrophobe, 3000; approx. wt. % of hydrophile, 50%), Pluronic® P85 (ave. MW: 4600; approx. MW of hydrophobe, 2400; approx. wt. % of hydrophile, 50%), Pluronic® P75 (ave. MW: 4150; approx. MW of hydrophobe, 2100; approx. wt. % of hydrophile, 50%), Pluronic® P65 (ave. MW: 3400; approx. MW of hydrophobe, 1800; approx. wt. % of hydrophile, 50%), Pluronic® F127 (ave. MW: 12600; approx. MW of hydrophobe, 3600; approx. wt. % of hydrophile, 70%), Pluronic® F98 (ave. MW: 13000; approx. MW of hydrophobe, 2700; approx. wt. % of hydrophile, 80%), Pluronic® F87 (ave. MW: 7700; approx. MW of hydrophobe, 2400; approx. wt. % of hydrophile, 70%), Pluronic® F77 (ave. MW: 6600; approx. MW of hydrophobe, 2100; approx. wt. % of hydrophile, 70%), Pluronic® F108 (ave. MW: 14600; approx. MW of hydrophobe, 3000; approx. wt. % of hydrophile, 80%), Pluronic® F98 (ave. MW: 13000; approx. MW of hydrophobe, 2700; approx. wt. % of hydrophile, 80%), Pluronic® F88 (ave. MW: 11400; approx. MW of hydrophobe, 2400; approx. wt. % of hydrophile, 80%), Pluronic® F68 (ave. MW: 8400; approx. MW of hydrophobe, 1800; approx. wt. % of hydrophile, 80%), Pluronic® F38 (ave. MW: 4700; approx. MW of hydrophobe, 900; approx. wt. % of hydrophile, 80%).

Reverse poloxamers which may be screened for their ability to enhance the immune response according to the present invention include, but are not limited to Pluronic® R 31R1 (ave. MW: 3250; approx. MW of hydrophobe, 3100; approx. wt. % of hydrophile, 10%), Pluronic® R 25R1 (ave. MW: 2700; approx. MW of hydrophobe, 2500; approx. wt. % of hydrophile, 10%), Pluronic® R 17R1 (ave. MW: 1900; approx. MW of hydrophobe, 1700; approx. wt. % of hydrophile, 10%), Pluronic® R 31R2 (ave. MW: 3300; approx. MW of hydrophobe, 3100; approx. wt. % of hydrophile, 20%), Pluronic® R 25R2 (ave. MW: 3100; approx. MW of hydrophobe, 2500; approx. wt. % of hydrophile, 20%), Pluronic® R 17R2 (ave. MW: 2150; approx. MW of hydrophobe, 1700; approx. wt. % of hydrophile, 20%), Pluronic® R 12R3 (ave. MW: 1800; approx. MW of hydrophobe, 1200; approx. wt. % of hydrophile, 30%), Pluronic® R 31R4 (ave. MW: 4150; approx. MW of hydrophobe, 3100; approx. wt. % of hydrophile, 40%), Pluronic® R 25R4 (ave. MW: 3600; approx. MW of hydrophobe, 2500; approx. wt. % of hydrophile, 40%), Pluronic® R 22R4 (ave. MW: 3350; approx. MW of hydrophobe, 2200; approx. wt. % of hydrophile, 40%), Pluronic® R 17R4 (ave. MW: 3650; approx. MW of hydrophobe, 1700; approx. wt. % of hydrophile, 40%), Pluronic® R 25R5 (ave. MW: 4320; approx. MW of hydrophobe, 2500; approx. wt. % of hydrophile, 50%), Pluronic® R 10R5 (ave. MW: 1950; approx. MW of hydrophobe, 1000; approx. wt. % of hydrophile, 50%), Pluronic® R 25R8 (ave. MW: 8550; approx. MW of hydrophobe, 2500; approx. wt. % of hydrophile, 80%), Pluronic® R 17R8 (ave. MW: 7000; approx. MW of hydrophobe, 1700; approx. wt. % of hydrophile, 80%), and Pluronic® R 10R8 (ave. MW: 4550; approx. MW of hydrophobe, 1000; approx. wt. % of hydrophile, 80%).

Other commercially available poloxamers which may be screened for their ability to enhance the immune response according to the present invention include compounds that are block copolymer of polyethylene and polypropylene glycol such as Synperonic® L121 (ave. MW: 4400), Synperonic® L122 (ave. MW: 5000), Synperonic® P104 (ave. MW: 5850), Synperonic® P105 (ave. MW: 6500), Synperonic® P123 (ave. MW: 5750), Synperonic® P85 (ave. MW: 4600) and Synperonic® P94 (ave. MW: 4600), in which L indicates that the surfactants are liquids, P that they are pastes, the first digit is a measure of the molecular weight of the polypropylene portion of the surfactant and the last digit of the number, multiplied by 10, gives the percent ethylene oxide content of the surfactant; and compounds that are nonylphenyl polyethylene glycol such as Synperonic® NP10 (nonylphenol ethoxylated surfactant—10% solution), Synperonic® NP30 (condensate of 1 mole of nonylphenol with 30 moles of ethylene oxide) and Synperonic® NP5 (condensate of 1 mole of nonylphenol with 5.5 moles of naphthalene oxide).

Other poloxamers which may be screened for their ability to enhance the immune response according to the present invention include: (a) a polyether block copolymer comprising an A-type segment and a B-type segment, wherein the A-type segment comprises a linear polymeric segment of relatively hydrophilic character, the repeating units of which contribute an average Hansch-Leo fragmental constant of about −0.4 or less and have molecular weight contributions between about 30 and about 500, wherein the B-type segment comprises a linear polymeric segment of relatively hydrophobic character, the repeating units of which contribute an average Hansch-Leo fragmental constant of about −0.4 or more and have molecular weight contributions between about 30 and about 500, wherein at least about 80% of the linkages joining the repeating units for each of the polymeric segments comprise an ether linkage; (b) a block copolymer having a polyether segment and a polycation segment, wherein the polyether segment comprises at least an A-type block, and the polycation segment comprises a plurality of cationic repeating units; and (c) a polyether-polycation copolymer comprising a polymer, a polyether segment and a polycationic segment comprising a plurality of cationic repeating units of formula —NH—R⁰, wherein R⁰ is a straight chain aliphatic group of 2 to 6 carbon atoms, which may be substituted, wherein said polyether segments comprise at least one of an A-type of B-type segment. See U.S. Pat. No. 5,656,611. Other poloxamers of interest include CRL1005 (12 kDa, 5% POE), CRL8300 (11 kDa, 5% POE), CRL2690 (12 kDa, 10% POE), CRL4505 (15 kDa, 5% POE) and CRL1415 (9 kDa, 10% POE).

Other auxiliary agents which may be screened for their ability to enhance the immune response according to the present invention include, but are not limited to, Acacia (gum arabic); the poloxyethylene ether R—O—(C₂H₄O)ₓ—H (BRIJ®), e.g., polyethylene glycol dodecyl ether (BRIJ® 35, x=23), polyethylene glycol dodecyl ether (BRIJ® 30, x=4), polyethylene glycol hexadecyl ether (BRIJ® 52 x=2), polyethylene glycol hexadecyl ether (BRIJ® 56, x=10), polyethylene glycol hexadecyl ether (BRIJ® 58P, x=20), polyethylene glycol octadecyl ether (BRIJ® 72, x=2), polyethylene glycol octadecyl ether (BRIJ® 76, x=10), polyethylene glycol octadecyl ether (BRIJ® 78P, x=20), polyethylene glycol oleyl ether (BRIJ® 92V, x=2), and polyoxyl 10 oleyl ether (BRIJ® 97, x=10); poly-D-glucosamine (chitosan); chlorbutanol; cholesterol; diethanolamine; digitonin; dimethylsulfoxide (DMSO), ethylenediamine tetraacetic acid (EDTA); glyceryl monosterate; lanolin alcohols; mono- and di-glycerides; monoethanolamine; nonylphenol polyoxyethylene ether (NP-40®); octylphenoxypolyethoxyethanol (NONIDET NP-40 from Amresco); ethyl phenol poly (ethylene glycol ether)ⁿ, n=11 (Nonidet® P40 from Roche); octyl phenol ethylene oxide condensate with about 9 ethylene oxide units (nonidet P40); IGEPAL CA 630® ((octyl phenoxy)polyethoxyethanol; structurally same as NONIDET NP-40); oleic acid; oleyl alcohol; polyethylene glycol 8000; polyoxyl 20 cetostearyl ether; polyoxyl 35 castor oil; polyoxyl 40 hydrogenated castor oil; polyoxyl 40 stearate; polyoxyethylene sorbitan monolaurate (polysorbate 20, or TWEEN-20®; polyoxyethylene sorbitan monooleate (polysorbate 80, or TWEEN-80®); propylene glycol diacetate; propylene glycol monstearate; protamine sulfate; proteolytic enzymes; sodium dodecyl sulfate (SDS); sodium monolaurate; sodium stearate; sorbitan derivatives (SPAN®), e.g., sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85); 2,6,10,15,19,23-hexamethyl-2,6,10,14,18,22-tetracosa-hexaene (squalene); stachyose; stearic acid; sucrose; surfactin (lipopeptide antibiotic from Bacillus subtilis); dodecylpoly(ethyleneglycolether)₉ (Thesit®) MW 582.9; octyl phenol ethylene oxide condensate with about 9-10 ethylene oxide units (Triton X-100™); octyl phenol ethylene oxide condensate with about 7-8 ethylene oxide units (Triton X-114™); tris(2-hydroxyethyl)amine (trolamine); and emulsifying wax.

In certain adjuvant compostions, the adjuvant is a cytokine A composition of the present invention can comprise one or more cytokines, chemokines, or compounds that induce the production of cytokines and chemokines, or a polynucleotide encoding one or more cytokines, chemokines, or compounds that induce the production of cytokines and chemokines Examples include, but are not limited to, granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 10 (IL-10), interleukin 12 (IL-12), interleukin 15 (IL-15), interleukin 18 (IL-18), interferon alpha (IFNα), interferon beta (IFNβ), interferon gamma (IFNγ), interferon omega (IFNθ), interferon tau (IFNτ), interferon gamma inducing factor I (IGIF), transforming growth factor beta (TGF-β), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

In certain compositions of the present invention, the polynucleotide construct may be complexed with an adjuvant composition comprising (±)-N-(3-aminopropyl)-N,N-dimethyl-2,3-bis(syn-9-tetradeceneyloxy)-1-propanaminium bromide (GAP-DMORIE). The composition may also comprise one or more co-lipids, e.g., 1,2-dioleoyl-sn-glycero-3-phosphoethanolamine (DOPE),1,2-diphytanoyl-sn-glycero-3-phosphoethanolamine (DPyPE), and/or 1,2-dimyristoyl-glycer-3-phosphoethanolamine (DMPE). An adjuvant composition comprising GAP-DMORIE and DPyPE at a 1:1 molar ratio is referred to herein as Vaxfectin® adjuvant. See, e.g., PCT Publication No. WO 00/57917.

In other embodiments, the polynucleotide itself may function as an adjuvant as is the case when the polynucleotides of the invention are derived, in whole or in part, from bacterial DNA. Bacterial DNA containing motifs of unmethylated CpG-dinucleotides (CpG-DNA) triggers innate immune cells in vertebrates through a pattern recognition receptor (including toll receptors such as TLR 9) and thus possesses potent immunostimulatory effects on macrophages, dendritic cells and B-lymphocytes. See, e.g., Wagner, H., Curr. Opin. Microbiol. 5:62-69 (2002); Jung, J. et al., J. Immunol. 169: 2368-73 (2002); see also Klinman, D. M. et al., Proc. Natl. Acad. Sci. U.S.A. 93:2879-83 (1996). Methods of using unmethylated CpG-dinucleotides as adjuvants are described in, for example, U.S. Pat. Nos. 6,207,646, 6,406,705 and 6,429,199.

The ability of an adjuvant to increase the immune response to an antigen is typically manifested by a significant increase in immune-mediated protection. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th₂ response into a primarily cellular, or Th₁ response.

Nucleic acid molecules and/or polynucleotides of the present invention, e.g., plasmid DNA, mRNA, linear DNA or oligonucleotides, may be solubilized in any of various buffers. Suitable buffers include, for example, phosphate buffered saline (PBS), normal saline, Tris buffer, and sodium phosphate (e.g., 150 mM sodium phosphate). Insoluble polynucleotides may be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art. For aqueous compositions used in vivo, sterile pyrogen-free water can be used. Such formulations will contain an effective amount of a polynucleotide together with a suitable amount of an aqueous solution in order to prepare pharmaceutically acceptable compositions suitable for administration to a human.

Compositions of the present invention can be formulated according to known methods. Suitable preparation methods are described, for example, in Remington's Pharmaceutical Sciences, 16th Edition, (A. Osol, ed., Mack Publishing Co., Easton, Pa. (1980)), and Remington's Pharmaceutical Sciences, 19th Edition, (A. R. Gennaro, ed., Mack Publishing Co., Easton, Pa. (1995)). Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives.

The following examples are included for purposes of illustration only and are not intended to limit the scope of the present invention, which is defined by the appended claims.

### EXAMPLES

### Materials and Methods

The following materials and methods apply generally to all the examples disclosed herein. Specific materials and methods are disclosed in each example, as necessary.

The practice of the present invention will employ, unless otherwise indicated, conventional techniques of cell biology, cell culture, molecular biology (including PCR), vaccinology, microbiology, recombinant DNA, and immunology, which are within the skill of the art. Such techniques are explained fully in the literature. See, for example, Molecular Cloning A Laboratory Manual, 2nd Ed., (Sambrook et al., ed., Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and in Ausubel et al., Current Protocols in Molecular Biology, (John Wiley and Sons, Baltimore, Md. 1989).

Gene Construction

Constructs of the present invention are constructed based on the sequence information provided herein or in the art utilizing standard molecular biology techniques, including, but not limited to, the following. First, a series complementary oligonucleotide pairs of 80-90 nucleotides each in length and spanning the length of the construct are synthesized by standard methods. These oligonucleotide pairs are synthesized such that upon annealing, they form double stranded fragments of 80-90 base pairs, containing cohesive ends. The single-stranded ends of each pair of oligonucleotides are designed to anneal with a single-stranded end of an adjacent oligonucleotide duplex. Several adjacent oligonucleotide pairs prepared in this manner are allowed to anneal, and approximately five to six adjacent oligonucleotide duplex fragments are then allowed to anneal together via the cohesive single stranded ends. This series of annealed oligonucleotide duplex fragments is then ligated together and cloned into a suitable plasmid, such as the TOPO® vector available from Invitrogen Corporation, Carlsbad, Calif. The construct is then sequenced by standard methods. Constructs prepared in this manner, comprising 5 to 6 adjacent 80 to 90 base pair fragments ligated together, i.e., fragments of about 500 base pairs, are prepared, such that the entire desired sequence of the construct is represented in a series of plasmid constructs. The inserts of these plasmids are then cut with appropriate restriction enzymes and ligated together to form the final construct. The final construct is then cloned into a standard bacterial cloning vector, and sequenced. The oligonucleotides and primers referred to herein can easily be designed by a person of skill in the art based on the sequence information provided herein and in the art, and such can be synthesized by any of a number of commercial nucleotide providers, for example Retrogen, San Diego, Calif., and GENEART, Regensburg, Germany.

Plasmid Vectors

Constructs of the present invention can be inserted, for example, into eukaryotic expression vectors VR1012 or VR10551. These vectors are built on a modified pUC18 background (see Yanisch-Perron, C., et al., Gene 33:103-119 (1985)), and contain a kanamycin resistance gene, the human cytomegalovirus immediate early promoter/enhancer and intron A, and the bovine growth hormone transcription termination signal, and a polylinker for inserting foreign genes. See Hartikka, J., et al., Hum. Gene Ther. 7:1205-1217 (1996). However, other standard commercially available eukaryotic expression vectors may be used in the present invention, including, but not limited to: plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.).

An optimized backbone plasmid, termed VR10551, has minor changes from the VR1012 backbone described above. The VR10551 vector is derived from and similar to VR1012 in that it uses the human cytomegalovirus immediate early (hCMV-IE) gene enhancer/promoter and 5′ untranslated region (UTR), including the hCMV-IE Intron A. The changes from the VR1012 to the VR10551 include some modifications to the multiple cloning site, and a modified rabbit β globin 3′ untranslated region/polyadenylation signal sequence/transcriptional terminator has been substituted for the same functional domain derived from the bovine growth hormone gene.

Plasmid DNA Purification

Plasmid DNA may be transformed into competent cells of an appropriate Escherichia coli strain (including but not limited to the DH5α strain) and highly purified covalently closed circular plasmid DNA was isolated by a modified lysis procedure (Horn, N. A., et al., Hum. Gene Ther. 6:565-573 (1995)) followed by standard double CsCl-ethidium bromide gradient ultracentrifugation (Sambrook, J., et al., Molecular Cloning: A Laboratory Manual, 2nd Ed., Cold Spring Harbor Laboratory Press, Plainview, N.Y. (1989)). Alternatively, plasmid DNAs are purified using Giga columns from Qiagen (Valencia, Calif.) according to the kit instructions. All plasmid preparations were free of detectable chromosomal DNA, RNA and protein impurities based on gel analysis and the bicinchoninic protein assay (Pierce Chem. Co., Rockford Ill.). Endotoxin levels were measured using Limulus Amebocyte Lysate assay (LAL, Associates of Cape Cod, Falmouth, Mass.) and were less than 0.6 Endotoxin Units/mg of plasmid DNA. The spectrophotometric A₂₆₀/A₂₈₀ ratios of the DNA solutions were typically above 1.8. Plasmids were ethanol precipitated and resuspended in an appropriate solution, e.g., 150 mM sodium phosphate (for other appropriate excipients and auxiliary agents, see U.S. patent application Publication 2002/0019358, published Feb. 14, 2002). DNA was stored at −20° C. until use. DNA was diluted by mixing it with 300 mM salt solutions and by adding appropriate amount of USP water to obtain 1 mg/ml plasmid DNA in the desired salt at the desired molar concentration.

Plasmid Expression in Mammalian Cell Lines

The expression plasmids are analyzed in vitro by transfecting the plasmids into a well characterized mouse melanoma cell line (VM-92, also known as UM-449). See, e.g., Wheeler, C. J., Sukhu, L., Yang, G., Tsai, Y., Bustamente, C., Felgner, P. Norman, J & Manthorpe, M. “Converting an Alcohol to an Amine in a Cationic Lipid Dramatically Alters the Co-lipid Requirement, Cellular Transfection Activity and the Ultrastructure of DNA-Cytofectin Complexes,” Biochim. Biophys. Acta. 1280:1-11 (1996). Other well-characterized human cell lines can also be used, e.g. MRC-5 cells, ATCC Accession No. CCL-171 or human rhabdomyosarcoma cell line RD (ATCC CCL-136). The transfection is performed using cationic lipid-based transfection procedures well known to those of skill in the art. Other transfection procedures are well known in the art and may be used, for example electroporation and calcium chloride-mediated transfection (Graham F. L. and A. J. van der Eb Virology 52:456-67 (1973)). Following transfection, cell lysates and culture supernatants of transfected cells are evaluated to compare relative levels of expression of measles virus antigen proteins. The samples are assayed by western blots and ELISAs, using available polyclonal and/or monoclonal antibodies (available, e.g., from Research Diagnostics Inc., Flanders N.J.), so as to compare both the quality and the quantity of expressed antigen.

In addition to plasmids encoding single measles virus proteins, single plasmids which contain two or more measles virus coding regions are constructed according to standard methods. For example, a polycistronic construct, where two or more measles virus coding regions are transcribed as a single transcript in eukaryotic cells may be constructed by separating the various coding regions with IRES sequences. Alternatively, two or more coding regions may be inserted into a single plasmid, each with their own promoter sequence.

Codon Optimization Algorithm

The following is an outline of the algorithm used to derive human codon-optimized sequences of measles antigens.

Back Translation

Starting with the amino acid sequence, one can either (a) manually backtranslate using the human codon usage table from http://www.kazusa.or.jp/codon/

Homo sapiens [gbpri]: 55194 CDS's (24298072 codons)

Fields: [triplet] [frequency: per thousand] ([number])

TABLE 6

| | | | | | | | |
|---|---|---|---|---|---|---|---|
| UUU | 17.1(415589) | UCU | 14.7(357770) | UAU | 12.1(294182) | UGU | 10.0(243198) |
| UUC | 20.6(500964) | UCC | 17.6(427664) | UAC | 15.5(377811) | UGC | 12.2(297010) |
| UUA | 7.5(182466) | UCA | 12.0(291788) | UAA | 0.7(17545) | UGA | 1.5(36163) |
| UUG | 12.6(306793) | UCG | 4.4(107809) | UAG | 0.6(13416) | UGG | 12.7(309683) |
| CUU | 13.0(315804) | CCU | 17.3(419521) | CAU | 10.5(255135) | CGU | 4.6(112673) |
| CUC | 19.8(480790) | CCC | 20.1(489224) | CAC | 15.0(364828) | CGC | 10.7(259950) |
| CUA | 7.8(189383) | CCA | 16.7(405320) | CAA | 12.0(292745) | CGA | 6.3(152905) |
| CUG | 39.8(967277) | CCG | 6.9(168542) | CAG | 34.1(827754) | CGG | 11.6(281493) |
| AUU | 16.1(390571) | ACU | 13.0(315736) | AAU | 16.7(404867) | AGU | 11.9(289294) |
| AUC | 21.6(525478) | ACC | 19.4(471273) | AAC | 19.5(473208) | AGC | 19.3(467869) |
| AUA | 7.7(186138) | ACA | 15.1(366753) | AAA | 24.1(585243) | AGA | 11.5(278843) |
| AUG | 22.2(538917) | ACG | 6.1(148277) | AAG | 32.2(781752) | AGG | 11.4(277693) |
| GUU | 11.0(266493) | GCU | 18.6(451517) | GAU | 21.9(533009) | GGU | 10.8(261467) |
| GUC | 14.6(354537) | GCC | 28.4(690382) | GAC | 25.6(621290) | GGC | 22.5(547729) |
| GUA | 7.2(174572) | GCA | 16.1(390964) | GAA | 29.0(703852) | GGA | 16.4(397574) |
| GUG | 28.4(690428) | GCG | 7.5(181803) | GAG | 39.9(970417) | GGG | 16.3(396931) |

* Coding GC 52.45% 1st letter GC 56.04% 2nd letter GC 42.37% 3rd letter GC 58.93%

Or (b) log on to www.syntheticgenes.com and use the backtranslation tool, as follows:

(1) Under Protein tab, paste amino acid sequence;

(2) Under download codon usage tab, highlight homo sapiens and then download CUT.

TABLE 7

| | | | | | | | |
|---|---|---|---|---|---|---|---|
| UUU | 17.1(415589) | UCU | 14.7(357770) | UAU | 12.1(294182) | UGU | 10.0(243198) |
| UUC | 20.6(500964) | UCC | 17.6(427664) | UAC | 15.5(377811) | UGC | 12.2(297010) |
| UUA | 7.5(182466) | UCA | 12.0(291788) | UAA | 0.7(17545) | UGA | 1.5(36163) |
| UUG | 12.6(306793) | UCG | 4.4(107809) | UAG | 0.6(13416) | UGG | 12.7(309683) |
| CUU | 13.0(315804) | CCU | 17.3(419521) | CAU | 10.5(255135) | CGU | 4.6(112673) |
| CUC | 19.8(480790) | CCC | 20.1(489224) | CAC | 15.0(364828) | CGC | 10.7(259950) |
| CUA | 7.8(189383) | CCA | 16.7(405320) | CAA | 12.0(292745) | CGA | 6.3(152905) |
| CUG | 39.8(967277) | CCG | 6.9(168542) | CAG | 34.1(827754) | CGG | 11.6(281493) |
| AUU | 16.1(390571) | ACU | 13.0(315736) | AAU | 16.7(404867) | AGU | 11.9(289294) |
| AUC | 21.6(525478) | ACC | 19.4(471273) | AAC | 19.5(473208) | AGC | 19.3(467869) |
| AUA | 7.7(186138) | ACA | 15.1(366753) | AAA | 24.1(585243) | AGA | 11.5(278843) |
| AUG | 22.2(538917) | ACG | 6.1(148277) | AAG | 32.2(781752) | AGG | 11.4(277693) |
| GUU | 11.0(266493) | GCU | 18.6(451517) | GAU | 21.9(533009) | GGU | 10.8(261467) |
| GUC | 14.6(354537) | GCC | 28.4(690382) | GAC | 25.6(621290) | GGC | 22.5(547729) |
| GUA | 7.2(174572) | GCA | 16.1(390964) | GAA | 29.0(703852) | GGA | 16.4(397574) |
| GUG | 28.4(690428) | GCG | 7.5(181803) | GAG | 39.9(970417) | GGG | 16.3(396931) |

(3) Hit Apply button.

(4) Under Optimize TAB, open General TAB.

(5) Check use only most frequent codon box.

(6) Hit Apply button.

(7) Under Optimize TAB, open Motif TAB.

(8) Load desired cloning restriction sites into bad motifs; load any undesirable sequences, such as Pribnow Box sequences (TATAA), Chi sequences (GCTGGCGG), and restriction sites into bad motifs.

(9) Under Output TAB, click on Start box. Output will include sequence, motif search results (under Report TAB), and codon usage report.

The program did not always use the most frequent codon for amino acids such as cysteine proline, and arginine. To change this, go back to the Edit CUT TAB and manually drag the rainbow colored bar to 100% for the desired codon. Then re-do start under the Output TAB.

The use of CGG for arginine can lead to very high GC content, so AGA can be used for arginine as an alternative. The difference in codon usage is 11.6 per thousand for CGG vs. 11.5 per thousand for AGA.

Splice Donor and Acceptor Site Search

(1) Log on to Berkeley Drosophila Genome Project Website at http://www.fruitfly.org/seg_tools/spice.html\

(2) Check boxes for Human or other and both splice sites.

(3) Select minimum scores for 5′ and 3′ splice sites between 0 and 1.
Used the default setting at 0.4 where:
Default minimum score is 0.4, where:

| | % splice sites | % false |
|---|---|---|
| | recognized | positives |
| Human 5′ Splice sites | 93.2% | 5.2% |
| Human 3′ Splice sites | 83.8% | 3.1% |

(4) Paste in sequence.

(5) Submit.

(6) Based on predicted donors or acceptors, change the individual codons until the sites are no longer predicted.

Add in 5′ and 3′ Sequences.

On the 5′ end of the gene sequence, the restriction enzyme site and Kozak sequence (gccacc) was added before ATG. On 3′ end of the sequence, tca was added following the stop codon (tga on opposite strand) and then a restriction enzyme site. The GC content and Open Reading Frames were then checked in SEC Central.

Preparation of Vaccine Formulations

Plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, are formulated with the poloxamer CRL 1005 and BAK (Benzalkonium chloride 50% solution, available from Ruger Chemical Co. Inc.) by the following methods. Specific final concentrations of each component of the formulae are described in the following methods, but for any of these methods, the concentrations of each component may be varied by basic stoichiometric calculations known by those of ordinary skill in the art to make a final solution having the desired concentrations.

For example, the concentration of CRL 1005 is adjusted depending on, for example, transfection efficiency, expression efficiency, or immunogenicity, to achieve a final concentration of between about 1 mg/ml to about 75 mg/ml, for example, about 1 mg/ml, about 2 mg/ml, about 3 mg/ml, about 4 mg/ml, about 5 mg/ml, about 6.5 mg/ml, about 7 mg/ml, about 7.5 mg/ml, about 8 mg/ml, about 9 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, or about 75 mg/ml of CRL 1005.

Similarly the concentration of DNA is adjusted depending on many factors, including the amount of a formulation to be delivered, the age and weight of the subject, the delivery method and route and the immunogenicity of the antigen being delivered. In general, formulations of the present invention are adjusted to have a final concentration from about 1 ng/ml to about 30 mg/ml of plasmid (or other polynucleotide). For example, a formulation of the present invention may have a final concentration of about 1 ng/ml, about 5 ng/ml, about 10 ng/ml, about 50 ng/ml, about 100 ng/ml, about 500 ng/ml, about 1 μg/ml, about 5 μg/ml, about 10 μg/ml, about 50 μg/ml, about 200 μg/ml, about 400 μg/ml, about 600 μg/ml, about 800 μg/ml, about 1 mg/ml, about 2 mg/ml, about 2.5, about 3 mg/ml, about 3.5, about 4 mg/ml, about 4.5, about 5 mg/ml, about 5.5 mg/ml, about 6 mg/ml, about 7 mg/ml, about 8 mg/ml, about 9 mg/ml, about 10 mg/ml, about 20 mg/ml, or about 30 mg mg/ml of a plasmid.

Certain formulations of the present invention include a cocktail of plasmids of the present invention, e.g., comprising coding regions encoding measles virus proteins HA and F and optionally, plasmids encoding immunity enhancing proteins, e.g., cytokines Various plasmids desired in a cocktail are combined together in PBS or other diluent prior to the addition to the other ingredients. Furthermore, plasmids may be present in a cocktail at equal proportions, or the ratios may be adjusted based on, for example, relative expression levels of the antigens or the relative immunogenicity of the encoded antigens. Thus, various plasmids in the cocktail may be present in equal proportion, or up to twice or three times as much of one plasmid may be included relative to other plasmids in the cocktail.

Additionally, the concentration of BAK may be adjusted depending on, for example, a desired particle size and improved stability. Indeed, in certain embodiments, formulations of the present invention include CRL 1005 and DNA, but are free of BAK. In general BAK-containing formulations of the present invention are adjusted to have a final concentration of BAK from about 0.05 mM to about 0.5 mM. For example, a formulation of the present invention may have a final BAK concentration of about 0.05 mM, 0.1 mM, 0.2 mM, 0.3 mM, 0.4 mM or 0.5 mM.

The total volume of the formulations produced by the methods below may be scaled up or down, by choosing apparatus of proportional size. Finally, in carrying out any of the methods described below, the three components of the formulation, BAK, CRL 1005, and plasmid DNA, may be added in any order. In each of these methods described below the term “cloud point” refers to the point in a temperature shift, or other titration, at which a clear solution becomes cloudy, i.e., when a component dissolved in a solution begins to precipitate out of solution.

Thermal Cycling of a Pre-Mixed Formulation

This example describes the preparation of a formulation comprising 0.3 mM BAK, 7.5 mg/ml CRL 1005, and 5 mg/ml of DNA in a total volume of 3.6 ml. The ingredients are combined together at a temperature below the cloud point and then the formulation is thermally cycled to room temperature (above the cloud point) several times.

A 1.28 mM solution of BAK is prepared in PBS, 846 μl of the solution is placed into a 15 ml round bottom flask fitted with a magnetic stirring bar, and the solution is stirred with moderate speed, in an ice bath on top of a stirrer/hotplate (hotplate off) for 10 minutes. CRL 1005 (27 μl) is then added using a 100 μl positive displacement pipette and the solution is stirred for a further 60 minutes on ice. Plasmids comprising codon-optimized coding regions encoding, for example, NP, M1, and M2 as described herein, and optionally, additional plasmids comprising codon-optimized or non-codon-optimized coding regions encoding, e.g., additional measles virus proteins, and or other proteins, e.g., cytokines, are mixed together at desired proportions in PBS to achieve 6.4 mg/ml total DNA. This plasmid cocktail is added drop wise, slowly, to the stirring solution over 1 min using a 5 ml pipette. The solution at this point (on ice) is clear since it is below the cloud point of the poloxamer and is further stirred on ice for 15 min. The ice bath is then removed, and the solution is stirred at ambient temperature for 15 minutes to produce a cloudy solution as the poloxamer passes through the cloud point.

The flask is then placed back into the ice bath and stirred for a further 15 minutes to produce a clear solution as the mixture is cooled below the poloxamer cloud point. The ice bath is again removed and the solution stirred at ambient temperature for a further 15 minutes. Stirring for 15 minutes above and below the cloud point (total of 30 minutes), is defined as one thermal cycle. The mixture is cycled six more times. The resulting formulation may be used immediately, or may be placed in a glass vial, cooled below the cloud point, and frozen at −80° C. for use at a later time.

Animal Immunizations

The immunogenicity of the various measles virus expression products encoded by the codon-optimized polynucleotides described herein are initially evaluated based on each plasmid's ability to mount an immune response in vivo. Plasmids are tested individually and in combinations by injecting single constructs as well as multiple constructs. Immunizations are initially carried out in animals, such as mice, rabbits, goats, sheep, non-human primates, or other suitable animal, by intramuscular (IM) or intradermal (ID) injections. Blood is collected from immunized animals, and the antigen specific antibody response is quantified by ELISA assay using purified immobilized antigen proteins in a protein—immunized subject antibody—anti-species antibody type assay, according to standard protocols. The tests of immunogenicity further include measuring antibody titer, neutralizing antibody titer, T-cell proliferation, T-cell secretion of cytokines, cytolytic T cell responses, and by direct enumeration of antigen specific CD4+ and CD8+ T-cells. Correlation to protective levels of the immune responses in humans are made according to methods well known by those of ordinary skill in the art.

A. DNA Formulations

Plasmid DNA is formulated with a poloxamer. Alternatively, plasmid DNA is prepared and dissolved at a concentration of about 0.1 mg/ml to about 10 mg/ml, preferably about 1 mg/ml, in PBS with or without transfection-facilitating cationic lipids, e.g., DMRIE/DOPE at a 4:1 DNA:lipid mass ratio. Alternative DNA formulations include 150 mM sodium phosphate instead of PBS, adjuvants, e.g., Vaxfectin® at a 4:1 DNA:Vaxfectin® mass ratio, mono-phosphoryl lipid A (detoxified endotoxin) from S. minnesota (MPL) and trehalosedicorynomycolateAF (TDM), in 2% oil (squalene)-Tween 80-water (MPL+TDM, available from Sigma/Aldrich, St. Louis, Mo., (catalog # M6536)), a solubilized mono-phosphoryl lipid A formulation (AF, available from Corixa), or (±)-N-(3-Acetoxypropyl)-N,N-dimethyl-2,3-bis(octyloxy)-1-propanaminium chloride (compound # VC1240) (see Shriver, J. W. et al., Nature 415:331-335 (2002), and P.C.T. Publication No. WO 02/00844 A2.

B. Animal Immunizations

Plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, are injected into BALB/c mice as single plasmids or as cocktails of two or more plasmids, as either DNA in PBS or formulated with the poloxamer-based delivery system: 2 mg/ml DNA, 3 mg/ml CRL 1005, and 0.1 mM BAK. Groups of 10 mice are immunized three times, at biweekly intervals, and serum is obtained to determine antibody titers to each of the antigens. Groups are also included in which mice are immunized with a trivalent preparation, containing each of the three plasmid constructs in equal mass.

The immunization schedule is as follows:

| | |
|---|---|
| Day 3 | Pre-bleed |
| Day 0 | Plasmid injections, intramuscular, bilateral |
| | in rectus femoris, 5-50 μg/leg |
| Day 21 | Plasmid injections, intramuscular, bilateral |
| | in rectus femoris, 5-50 μg/leg |
| Day 49 | Plasmid injections, intramuscular, bilateral |
| | in rectus femoris, 5-50 μg/leg |
| Day 59 | Serum collection |

Serum antibody titers are determined by ELISA with recombinant proteins, peptides or transfection supernatants and lysates from transfected VM-92 cells live, inactivated, or lysed virus.

C. Immunization of Mice with Vaccine Formulations Using a Vaxfectin® Adjuvant

Vaxfectin® adjuvant (a 1:1 molar ratio of the cationic lipid VC1052 and the neutral co-lipid DPyPE) is a synthetic cationic lipid formulation which has shown promise for its ability to enhance antibody titers against when administered with DNA intramuscularly to mice.

In mice, intramuscular injection of Vaxfectin® formulated with measles virus DNA increased antibody titers up to 20-fold to levels that could not be reached with DNA alone. In rabbits, complexing DNA with Vaxfectin® enhanced antibody titers up to 50-fold.

Vaxfectin® mixtures are prepared by mixing chloroform solutions of VC1052 cationic lipid with chloroform solutions of DPyPE neutral co-lipid. Dried films are prepared in 2 ml sterile glass vials by evaporating the chloroform under a stream of nitrogen, and placing the vials under vacuum overnight to remove solvent traces. Each vial contains 1.5 μmole each of VC1052 and DPyPE. Liposomes are prepared by adding sterile water followed by vortexing. The resulting liposome solution is mixed with DNA at a phosphate mole:cationic lipid mole ratio of 4:1.

Plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, are mixed together at desired proportions in PBS to achieve a final concentration of 1.0 mg/ml. The plasmid cocktail, as well as the controls, are formulated with Vaxfectin®. Groups of 5 BALB/c female mice are injected bilaterally in the rectus femoris muscle with 50 μl of DNA solution (100 μl total/mouse), on days 1 and 21 and 49 with each formulation. Mice are bled for serum on days 0 (prebleed), 20 (bleed 1), and 41 (bleed 2), and 62 (bleed 3), and up to 40 weeks post-injection. Antibody titers to the various measles virus proteins encoded by the plasmid DNAs are measured by ELISA.

Cytolytic T-cell responses are measured as described in Hartikka et al. “Vaxfectin Enhances the Humoral Response to Plasmid DNA-encoded Antigens,” Vaccine 19:1911-1923 (2001). Standard ELISPOT technology is used for the CD4+ and CD8+ T-cell assays.

D. Production of HA or F Antisera in Animals

Plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, are prepared according to the immunization scheme described above and injected into a suitable animal for generating polyclonal antibodies. Serum is collected and the antibody titered as above.

Monoclonal antibodies are also produced using hybridoma technology (Kohler, et al., Nature 256:495 (1975); Kohler, et al., Eur. J. Immunol. 6:511 (1976); Kohler, et al., Eur. J. Immunol. 6:292 (1976); Hammerling, et al., in Monoclonal Antibodies and T-Cell Hybridomas, Elsevier, N.Y., (1981), pp. 563-681. In general, such procedures involve immunizing an animal (preferably a mouse) as described above. The splenocytes of such mice are extracted and fused with a suitable myeloma cell line. Any suitable myeloma cell line may be employed in accordance with the present invention; however, it is preferable to employ the parent myeloma cell line (SP2O), available from the American Type Culture Collection, Rockville, Md. After fusion, the resulting hybridoma cells are selectively maintained in HAT medium, and then cloned by limiting dilution as described by Wands et al., Gastroenterology 80:225-232 (1981), incorporated herein by reference in its entirety. The hybridoma cells obtained through such a selection are then assayed to identify clones which secrete antibodies capable of binding the various measles virus proteins.

Alternatively, additional antibodies capable of binding to measles virus proteins described herein may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, various measles virus-specific antibodies are used to immunize an animal, preferably a mouse. The splenocytes of such an animal are then used to produce hybridoma cells, and the hybridoma cells are screened to identify clones which produce an antibody whose ability to bind to the measles virus protein-specific antibody can be blocked by the cognate measles virus protein. Such antibodies comprise anti-idiotypic antibodies to the measles virus protein-specific antibody and can be used to immunize an animal to induce formation of further measles virus-specific antibodies.

It will be appreciated that Fab and F(ab′)₂ and other fragments of the antibodies of the present invention may be used. Such fragments are typically produced by proteolytic cleavage, using enzymes such as papain (to produce Fab fragments) or pepsin (to produce F(ab′)₂ fragments). Alternatively, HA or F binding fragments can be produced through the application of recombinant DNA technology or through synthetic chemistry.

It may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi, et al., BioTechniques 4:214 (1986); Cabilly, et al., U.S. Pat. No. 4,816,567; Taniguchi, et al., EP 171496; Morrison, et al., EP 173494; Neuberger, et al., WO 8601533; Robinson, et al., WO 8702671; Boulianne, et al., Nature 312:643 (1984); Neuberger, et al., Nature 314:268 (1985).

These antibodies are used, for example, in diagnostic assays, as a research reagent, or to further immunize animals to generate measles virus-specific anti-idiotypic antibodies. Non-limiting examples of uses for anti-measles virus antibodies include use in Western blots, ELISA (competitive, sandwich, and direct), immunofluorescence, immunoelectron microscopy, radioimmunoassay, immunoprecipitation, agglutination assays, neutralization assays, immunodiffusion, immunoelectrophoresis, and epitope mapping (Weir, D. Ed. Handbook of Experimental Immunology, 4^{th} ed. Vols. I and II, Blackwell Scientific Publications (1986)).

Mucosal Vaccination and Electrically Assisted Plasmid Delivery

A. Mucosal DNA Vaccination

Plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, (100 μg/50 μl total DNA) are delivered to BALB/c mice at 0, 2 and 4 weeks via i.m., intranasal (i.n.), intravenous (i.v.), intravaginal (i.vag.), intrarectal (i.r.) or oral routes. The DNA is delivered unformulated or formulated with the cationic lipids DMRIE/DOPE (DD) or GAP-DLRIE/DOPE (GD). As endpoints, serum IgG titers against the various measles virus antigens are measured by ELISA and splenic T-cell responses are measured by antigen-specific production of IFN-gamma and IL-4 in ELISPOT assays. Standard chromium release assays are used to measure specific cytotoxic T lymphocyte (CTL) activity against the various measles virus antigens. Tetramer assays are used to detect and quantify antigen specific T-cells, with quantification being confirmed and phenotypic characterization accomplished by intracellular cytokine staining. In addition, IgG and IgA responses against the various measles virus antigens are analyzed by ELISA of vaginal washes.

B. Electrically-Assisted Plasmid Delivery

In vivo gene delivery may be enhanced through the application of brief electrical pulses to injected tissues, a procedure referred to herein as electrically-assisted plasmid delivery (EAPD). See, e.g., Aihara, H. & Miyazaki, J. Nat. Biotechnol. 16:867-70 (1998); Mir, L. M. et al., Proc. Natl. Acad. Sci. USA 96:4262-67 (1999); Hartikka, J. et al., Mol. Ther. 4:407-15 (2001); and Mir, L. M. and Rizzuto, G. et al., Hum Gene Ther 11:1891-900 (2000); Widera, G. et al, J. of Immuno. 164: 4635-4640 (2000). The use of electrical pulses for cell electropermeabilization has been used to introduce foreign DNA into prokaryotic and eukaryotic cells in vitro. Cell permeabilization can also be achieved locally, in vivo, using electrodes and optimal electrical parameters that are compatible with cell survival.

The electroporation procedure can be performed with various electroporation devices. These devices include external plate type electrodes or invasive needle/rod electrodes and can possess two electrodes or multiple electrodes placed in an array. Distances between the plate or needle electrodes can vary depending upon the number of electrodes, size of target area and treatment subject.

The TriGrid needle array, used in examples described herein, is a three electrode array comprising three elongate electrodes in the approximate shape of a geometric triangle. Needle arrays may include single, double, three, four, five, six or more needles arranged in various array formations. The electrodes are connected through conductive cables to a high voltage switching device that is connected to a power supply.

The electrode array is placed into the muscle tissue, around the site of nucleic acid injection, to a depth of approximately 3 mm to 3 cm. The depth of insertion varies depending upon the target tissue and size of patient receiving electroporation. After injection of foreign nucleic acid, such as plasmid DNA, and a period of time sufficient for distribution of the nucleic acid, square wave electrical pulses are applied to the tissue. The amplitude of each pulse ranges from about 100 volts to about 1500 volts, e.g., about 100 volts, about 200 volts, about 300 volts, about 400 volts, about 500 volts, about 600 volts, about 700 volts, about 800 volts, about 900 volts, about 1000 volts, about 1100 volts, about 1200 volts, about 1300 volts, about 1400 volts, or about 1500 volts or about 1-1.5 kV/cm, based on the spacing between electrodes. Each pulse has a duration of about 1 μs to about 1000 μs, e.g., about 1 μs, about 10 μs, about 50 μs, about 100 μs, about 200 μs, about 300 μs, about 400 μs, about 500 μs, about 600 μs, about 700 μs, about 800 μs, about 900 μs, or about 1000 μs, and a pulse frequency on the order of about 1-10 Hz. The polarity of the pulses may be reversed during the electroporation procedure by switching the connectors to the pulse generator. Pulses are repeated multiple times. The electroporation parameters (e.g. voltage amplitude, duration of pulse, number of pulses, depth of electrode insertion and frequency) will vary based on target tissue type, number of electrodes used and distance of electrode spacing, as would be understood by one of ordinary skill in the art.

Immediately after completion of the pulse regimen, subjects receiving electroporation can be optionally treated with membrane stabilizing agents to prolong cell membrane permeability as a result of the electroporation. Examples of membrane stabilizing agents include, but are not limited to, steroids (e.g. dexamethasone, methylprednisone and progesterone), angiotensin II and vitamin E. A single dose of dexamethasone, approximately 0.1 mg per kilogram of body weight, should be sufficient to achieve a beneficial affect.

EAPD techniques such as electroporation can also be used for plasmids contained in liposome formulations. The liposome-plasmid suspension is administered to the animal or patient and the site of injection is treated with a safe but effective electrical field generated, for example, by a TriGrid needle array. The electroporation may aid in plasmid delivery to the cell by destabilizing the liposome bilayer so that membrane fusion between the liposome and the target cellular structure occurs. Electroporation may also aid in plasmid delivery to the cell by triggering the release of the plasmid, in high concentrations, from the liposome at the surface of the target cell so that the plasmid is driven across the cell membrane by a concentration gradient via the pores created in the cell membrane as a result of the electroporation.

To test the effect of electroporation on therapeutic protein expression in non-human primates, male or female rhesus monkeys are given either 2 or 6 i.m. injections of plasmid constructs comprising codon-optimized and non-codon-optimized coding regions encoding HA or F; or alternatively coding regions (either codon-optimized or non-codon optimized) encoding various measles virus proteins or fragments, variants or derivatives either alone or as fusions with a carrier protein, e.g., HBcAg, as well as various controls, e.g., empty vector, (0.1 to 10 mg DNA total per animal). Target muscle groups include, but are not limited to, bilateral rectus fermoris, cranial tibialis, biceps, gastrocenemius or deltoid muscles. The target area is shaved and a needle array, comprising between 4 and 10 electrodes, spaced between 0.5-1.5 cm apart, is implanted into the target muscle. Once injections are complete, a sequence of brief electrical pulses are applied to the electrodes implanted in the target muscle using an Ichor TGP-2 pulse generator. The pulses have an amplitude of approximately 120-200V. The pulse sequence is completed within one second. During this time, the target muscle may make brief contractions or twitches. The injection and electroporation may be repeated.

Sera are collected from vaccinated monkeys at various time points. As endpoints, serum IgG titers against the various measles virus antigens are measured by ELISA and PBMC T-cell responses are measured by antigen-specific production of IFN-gamma and IL-4 in ELISPOT assays or by tetramer assays to detect and quantify antigen specific T-cells, with quantification being confirmed and phenotypic characterization accomplished by intracellular cytokine staining Standard chromium release assays are used to measure specific cytotoxic T lymphocyte (CTL) activity against the various MV antigens.

Vaxfectin®-formulated measles DNA vaccine encoding the hemagglutinin and fusion proteins completely protects juvenile and infant rhesus macaques from measles

### Materials and Methods

Animals. Six-week-old female BALB/c mice were purchased from Charles River Breeding Laboratories (Wilmington, Mass.). Twelve 2-year-old juvenile and four 1-month-old infant rhesus macaques (Macaca mulatta) born to measles naïve mothers were obtained from the Johns Hopkins Primate Breeding Facility. Monkeys were chemically restrained with ketamine (10-15 mg/kg) during procedures. All animals were maintained within the guidelines and studies were performed in accordance with experimental protocols approved by the Animal Care and Use Committee for Johns Hopkins University.

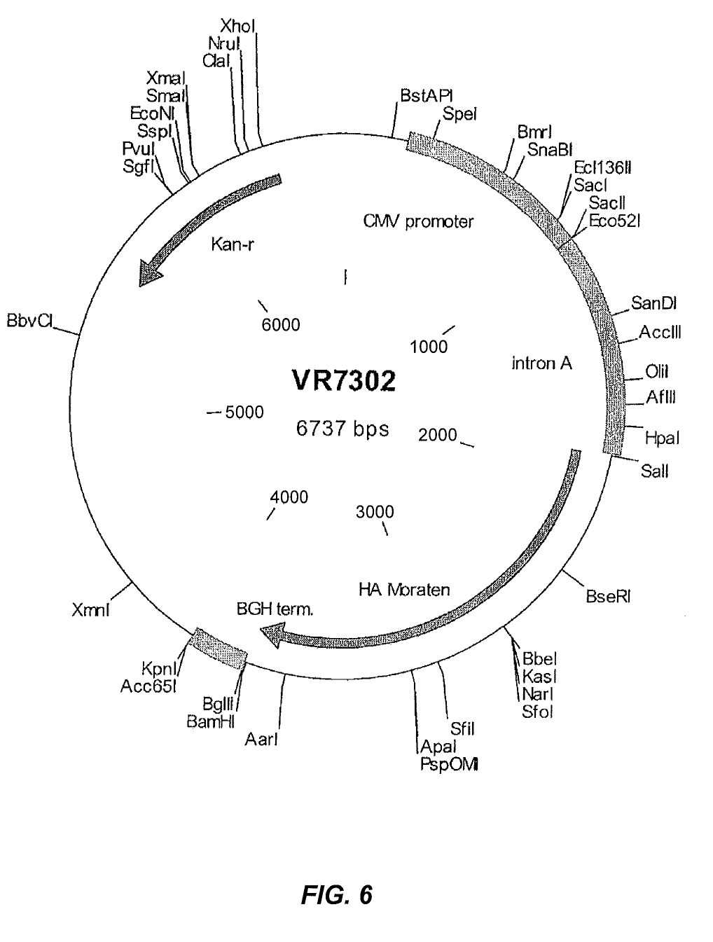

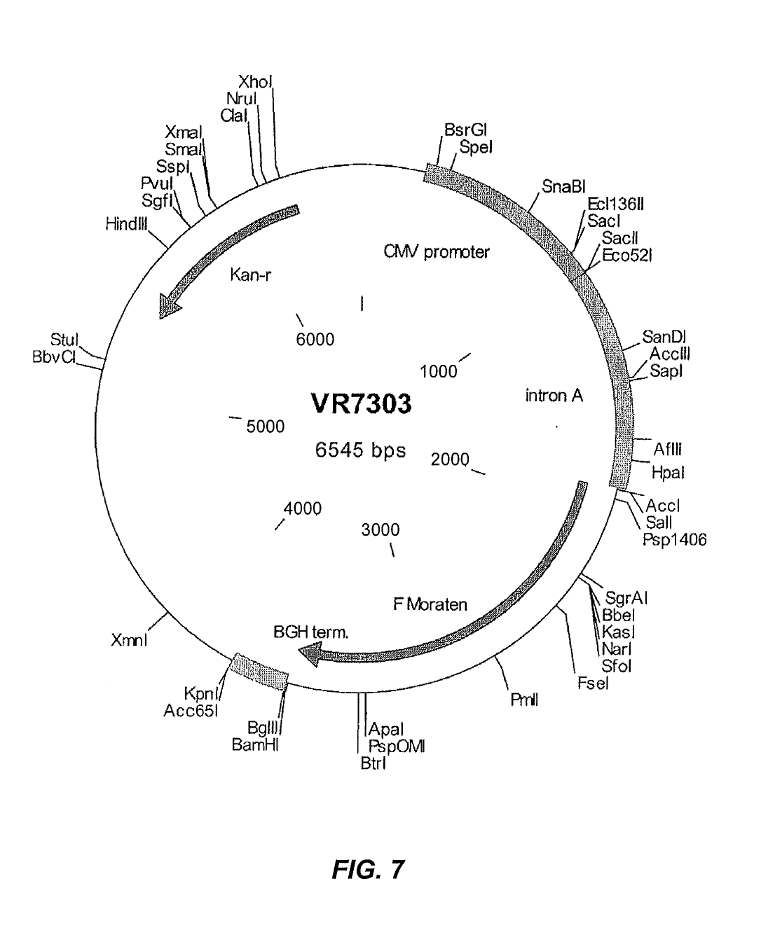

Vaccine. Coding nucleotide sequences for the HA and F antigens of the Moraten strain of MV were codon-optimized for expression in humans. Resulting DNA sequences were synthesized by GeneArt (Regensburg, Germany) and subcloned into expression plasmid VR1012 to create VR-HA (FIG. 6) and VR-F (FIG. 7). Coding nucleotide sequences for the HA and F antigens of the Edmonston strain of MV, from which Moraten was derived, were cloned into expression plasmid pGAT (from J. Peranen, Institute of Biotechnology, University of Helsinki, Finland) and into VR1012. Expression from VR-HA, pGAT-HA, VR-F and pGAT-F was confirmed by transient transfection of mouse VM92 cells followed by Western blot analysis. VR-HA and VR-F plasmids were formulated with Vaxfectin® as first described by Hartikka and co-workers Hartikka, J., et al., Vaccine, 19:1911-1923 (2001). Briefly, both GAP-DMORIE and DPyPE were resuspended in chloroform, mixed in 1:1 molar ratio, aliquoted into vials, and dried to create Vaxfectin® reagent dry lipid film. On the day of injection, the lipid film vials were resuspended in 1 mL 0.9% saline and diluted, if necessary. Plasmid DNA was prepared in 0.9% saline, 20 mM sodium phosphate, pH 7.2. Plasmid DNA was formulated with Vaxfectin® by gently streaming the lipid into pDNA of equal volume. All the required doses were prepared by formulating at 0.2 to 0.5 mg/mL range and diluting down to lower concentration as required. The final DNA: cationic lipid molar ratio was 4:1.

Immunization of mice. Groups of 6 mice received 1, 3, 10, 30 or 100 μg of Vaxfectin®-formulated VR-HA and VR-F with the codon-optimized Moraten sequences, 30 μg VR-HA and VR-F with the Edmonston sequences, 15 μg VR-HA, 15 μg VR-HA, 100 μg empty VR1012 plasmid or 100 μg pGAT-HA and pGAT-F without Vaxfectin® intramuscularly (IM). A second dose was delivered 4 weeks later. Mice were bled at 2 week intervals for measurement of antibody and spleens were collected at 4 weeks for assessment of the cellular immune response.

Vaccination and challenge of monkeys. Five juvenile rhesus macaques were immunized with Vaxfectin®-formulated VR-HA (1 mg) and VR-F (1 mg) IM. Five juvenile and 4 infant rhesus macaques were immunized with Vaxfectin®-formulated VR-HA (500 μg) and VR-F (500 μg) intradermally (ID, five sites for each plasmid). All monkeys were boosted 4 weeks later. One infant monkey died 10 weeks after immunization of unrelated causes. Juvenile monkeys were bled at 2 to 4 week intervals and infant monkeys were bled at monthly intervals after vaccination. Peripheral blood mononuclear cells (PBMCs) were separated from heparinized blood by Ficoll-Paque (Amersham Pharmacia) gradient centrifugation. Plasma was collected and stored at −20° C.

All monkeys were challenged intratracheally with 10⁴ tissue culture 50% infectious doses (TCID₅₀) of the wild type Bilthoven strain of MV (A. Osterhaus, Erasmas University, Rotterdam). Juvenile monkeys were challenged 15 months and infant macaques 12 months after first vaccination, along with two naïve juvenile monkeys. All monkeys were bled at regular intervals to monitor viremia and immune responses after challenge.

Virus assays. Viremia was assessed by cocultivation in triplicate of serial dilutions of PBMCs with B95-8 marmoset B cells in Dulbecco's modified Eagle's medium supplemented with 10% FBS, penicillin, and streptomycin. Wells were scored at 96 h for MV-positive syncytia. Data are reported as numbers of syncytia/10⁶ PBMC.

Antibody assays. Neutralizing antibodies were measured by the ability of serially diluted plasma to reduce plaque formation by the Chicago-1 strain of MV on Vero cells (i.e. plaque-reduction neutralization test, PRNT). The international standard serum 66/202 was included in all assays and data were normalized to that standard to calculate international units (IU) of neutralizing antibody per mL.

For enzyme immunoassays (EIAs), MV-infected Vero cell lysate (Advanced Biotechnologies, Columbia, Md.) was used (1.16 μg protein/well) to coat 96-well Maxisorp plates (Nunc, Rochester, N.Y.) and then incubated with serially diluted plasma overnight at 4° C. For mice, a horseradish peroxidase (HRP)-conjugated sheep antibody to mouse IgG (Amersham) was the secondary antibody and TMB (R&D Systems) was the substrate. A laboratory standard serum was included in each plate and data are presented as EIA units (EU) per mL. For monkeys, plasma was diluted 1:400 (IgG) or 1:100-200 (IgM) and an alkaline phosphatase-conjugated rabbit antibody to monkey IgG (Biomakor; Accurate Chemicals, Westbury, N.J.) or HRP-conjugated goat antibody to monkey IgM (Nordic, Capistrano Beach, Calif.) was used as the secondary antibody. Data are presented as optical density (OD) values.

To measure the avidity of MV-specific IgG, 50 μL of increasing concentrations (0-3.5 M) of ammonium thiocyanate (NH₄SCN) were added to EIA plates after incubation with plasma (1:100). Plates were washed and the rabbit anti-monkey IgG added as above. The avidity index was calculated as the concentration of NH₄SCN at which 50% of the bound antibody was eluted Nair, N., et al., J Infect Dis., 196:1339-1345 (2007)).

ELISPOT assays. For mice, spleen cells were harvested, incubated with RBC lysis buffer (Sigma), washed and suspended in RPMI supplemented with 10% FBS, 2 mM L-glutamine, penicillin and streptomycin. Multiscreen ELISPOT plates (Millipore) were coated with antibody to mouse IFN-γ or IL-4 (5 μg/mL, BD Pharmingen, San Diego, Calif.). Plates were washed, blocked with culture medium and 1-5×10⁵ splenocytes were added along with 1 μg/mL pooled MV HA or F peptides (20mers overlapping by 11 amino acids) Ota, M. O., et al., J. Infect. Dis., 195:1799-1807 (2007), Pan, C. H., et al., Proc. Natl. Acad. Sci. USA, 102:11581-11588 (2005), 5 μg/mL of concanavalin A (Con A; Sigma, St. Louis, Mo.) or medium. After 40 h incubation, plates were washed and incubated with 2 μg/mL biotinylated antibody to mouse IFN-γ or IL-4 for 2 h at 37° C. For monkeys, 1-5×10⁵ PBMCs were added to plates coated with antibody to human IFN-γ (2 μg/mL) or IL-4 (5 μg/mL) (BD Pharmingen) along with 1 μg/mL pooled MV HA or F peptides, 5 μg/mL of Con A, or medium. After 40 h at 37° C., plates were washed and incubated with 1 μg/mL biotinylated antibody to IFN-γ (Mabtech) or 2 μg/mL biotinylated antibody to IL-4 (PharMingen) at room temperature for 2 h. After washing, 50 μL of HRP-conjugated avidin (Research Laboratory Inc) were added into each well and incubated 1 h at 37° C. The assays were developed with 50 μL of stable diaminobenzidine solution (Invitrogen, Carlsbad, Calif.) for 10 min. Wells were scanned in an ImmunoSpot™ reader and analyzed using ImmunoSpot 2.0.5 software (C.T.L., Cleveland, Ohio). Data are presented as spot-forming cells (SFCs) per 10⁶ splenocytes or 10⁶ PBMCs after subtraction of the media control.

Statistical analysis. Student's unpaired t test or one-way ANOVA was used for comparison of responses between groups of monkeys using prism 4 software.

### Results

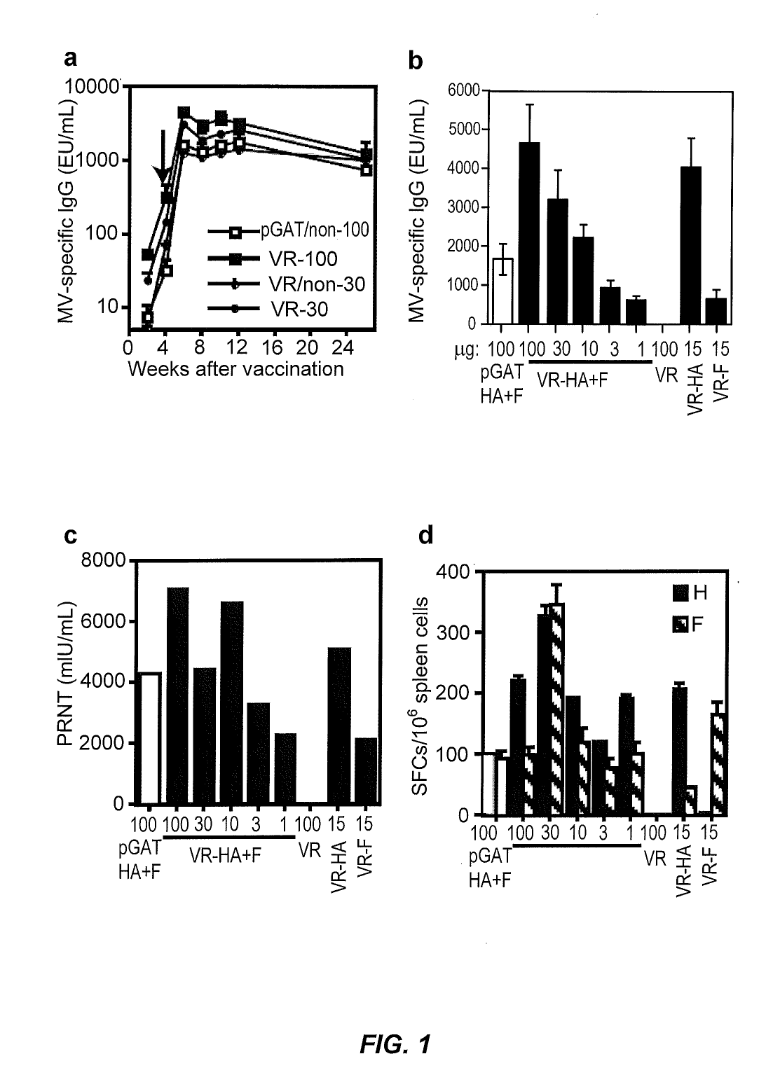

Immune responses in mice. To determine whether codon-optimization of the DNA HA and F sequences or Vaxfectin®-formulation improves immunogenicity of an MV DNA vaccine, mice were immunized with non-optimized or codon-optimized HA and F either formulated with (VR-HA and/or VR-F) or without (pGAT) Vaxfectin® (FIG. 1). MV-specific IgG was induced in all MV-immunized groups, reached a peak soon after the boost at 4 weeks, and was sustained at a high level through 26 weeks (FIG. 1A). The peak IgG titer was higher for 100 μg VR-HA+F (4646±413 EU/mL) than for 100 μg pGAT-HA+F (1660±392 EU/mL, p<0.05) and for 30 μg codon-optimized VR-HA+F (3182±807) than for 30 μg non-optimized VR-HA+F (1269±164). The antibody response to Vaxfectin®-formulated DNA was mostly dose-dependent for both EIA (FIG. 1B) and PRNT (FIG. 1C). VR-HA (15 μg) elicited a higher IgG and neutralizing antibody response than 15 μg VR-F or 30 μg VR-HA+F (FIG. 1B,C).

Spleen cell HA- and F-specific IFN-γ responses were assessed by ELISPOT assay. The highest response was in the group receiving 30 μg VR-HA+F (HA: 327±18; F: 347±31) (FIG. 1D) that was also higher than the response to 30 μg of non-optimized VR-HA+F (HA: 112±5; F: 75±19). MV-specific IL-4 production was not detected (data not shown). Based on these results in mice, subsequent studies in rhesus macaques used Vaxfectin®-formulated, codon-optimized VR-HA+F for vaccination.

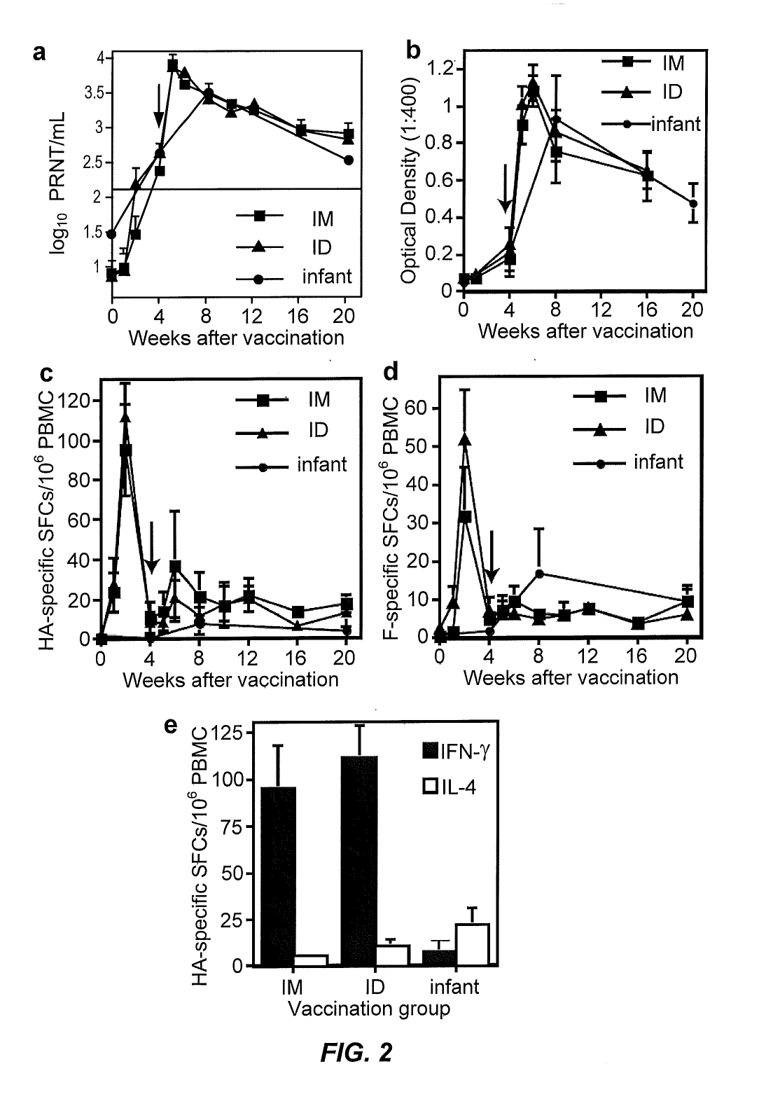

Immune responses in rhesus macaques. To evaluate route of administration, immunogenicity and protection from measles in nonhuman primates, groups of 5 juvenile rhesus macaques were immunized with 500 μg VR-HA+F ID or 1 mg VR-HA+F IM. Four 1 month-old infant monkeys received 500 μg VR-HA+F ID. Four weeks later, all monkeys were boosted with the same doses by the same routes. After the first dose, all juvenile and 3 of 4 infant monkeys had levels of MV-specific neutralizing antibodies above the generally recognized protective level (120 mIU/mL) (FIG. 2A). The maximum PRNT titers were achieved in juvenile macaques 2 weeks after the 4-week boost and were sustained above the protective level for over one year. Infant macaques could be assessed less frequently, but showed a similar pattern. The geometric mean peaks of neutralizing antibody for juvenile monkeys were 8710±2123 mIU/mL after IM administration and 7943+1425 mIU/mL after ID administration. For infant monkeys, the mean peak was 3561+1400 mIU/mL. There were no significant differences between IM and ID groups or juvenile and infant monkeys. MV-specific IgG EIA responses were induced in all VR-HA+F-immunized monkeys with a time course similar to the development of neutralizing antibody (FIG. 2B).

PBMC HA-specific (FIG. 2C) and F-specific (FIG. 2D) T cell responses were assessed using IFN-γ and IL-4 ELISPOT assays. All juvenile monkeys developed high IFN-γ and low IL-4 production (FIG. 2E). IFN-γ responses showed a peak in SFCs 2 weeks after vaccination, a slight increase after the boost and were detectable for over one year. Responses to HA were higher than to F in all juvenile monkeys. Peak HA-specific SFCs/10⁶ PBMC were 95±23 for IM and 112±17 for ID groups, while F-specific SFCs/10⁶ PBMC were 32±13 for IM (P=0.044) and 52±13 for ID (P=0.035) groups. For infant monkeys the IFN-γ responses to HA (15±7) and F (17±12) were similar. These young monkeys also developed IL-4 SFCs (HA: 22±9; F: 15±10), comparable to the IFN-γ SFC response (FIG. 2E).

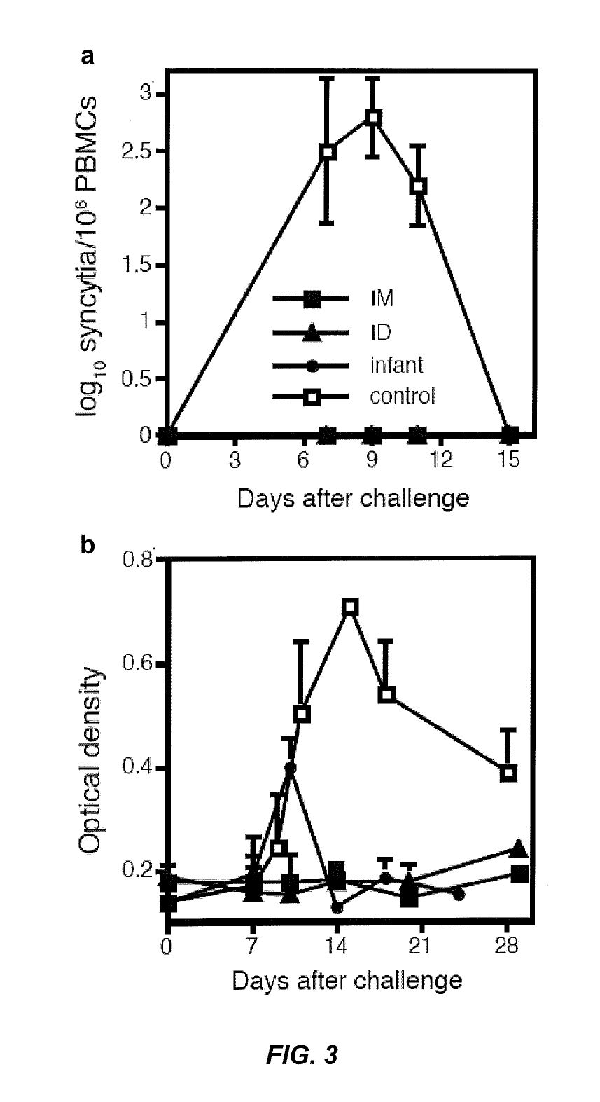

Protection of immunized monkeys from wild-type MV challenge. Twelve to 15 months after immunization, all vaccinated monkeys, plus two naïve monkeys, were challenged with wild-type MV. At the time of challenge, neutralizing antibody titers for all vaccinated juvenile monkeys (geometric mean=589±113 mIU/mL for IM; 527±105 mIU/mL for ID) and 2 of 3 infant monkeys (610, 203 and 57 mIU/mL) were predicted to be protective. Between 9 and 11 days after challenge, both naïve animals developed rashes on the face and trunk, while none of the vaccinated monkeys developed rashes. Naïve monkeys developed viremias with a mean peak of 10²⁵ TCID₅₀/10⁶ PBMC while none of the vaccinated juvenile or infant monkeys developed viremia detectable by cocultivation (FIG. 3A).

Antibody responses after challenge. Naïve monkeys showed a high MV-specific IgM response with peak OD values (0.71±0.02) at day 15 while juvenile monkeys immunized either IM or ID showed no change in IgM from baseline (OD=0.18±0.02) (FIG. 3B). Previously vaccinated infant monkeys had a transient IgM increase at day 10 (OD=0.4+0.06).

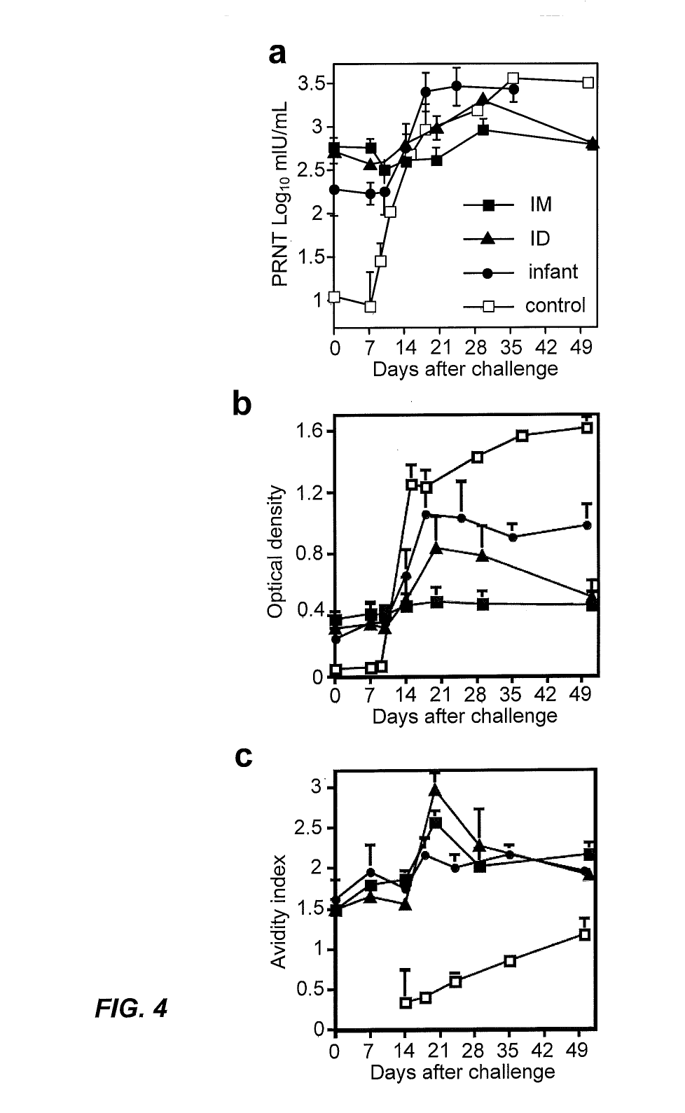

Neutralizing antibody responses in unvaccinated control animals appeared 10 days after challenge and continued to increase for months while titers increased only slightly in juvenile monkeys vaccinated either IM or ID (FIG. 4A). Neutralizing antibodies increased 10-fold in infant monkeys. All vaccinated monkeys had detectable MV-specific IgG measured by EIA before challenge with mean ODs of 0.377±0.05 for the juvenile IM group, 0.316±0.03 for the juvenile ID group and 0.25±0.07 for the infant ID group (FIG. 4B). After challenge, IgG levels increased minimally (0.492±0.09, day 20) for juvenile monkeys immunized IM, while they increased to 0.835±0.21 (day 20) in the ID group and to 1.057±0.15 (day 18) for infant monkeys.

All vaccinated monkeys showed a high avidity index for MV-specific IgG before challenge with a mean of 1.5±0.14 for juvenile monkeys immunized IM, 1.5±0.03 for juvenile monkeys immunized ID and 1.6±0.24 for infant monkeys immunized ID (FIG. 4C). After challenge, IgG avidity increased in all vaccinated monkeys and reached a peak 18-20 days after challenge and then decreased and plateaued above the prechallenge values (2.2±0.14 for IM; 1.9±0.1 for ID; 2.0±0.04 for infants). The unvaccinated control monkeys showed a slow rise in avidity that was 1.2±0.2 at day 50.

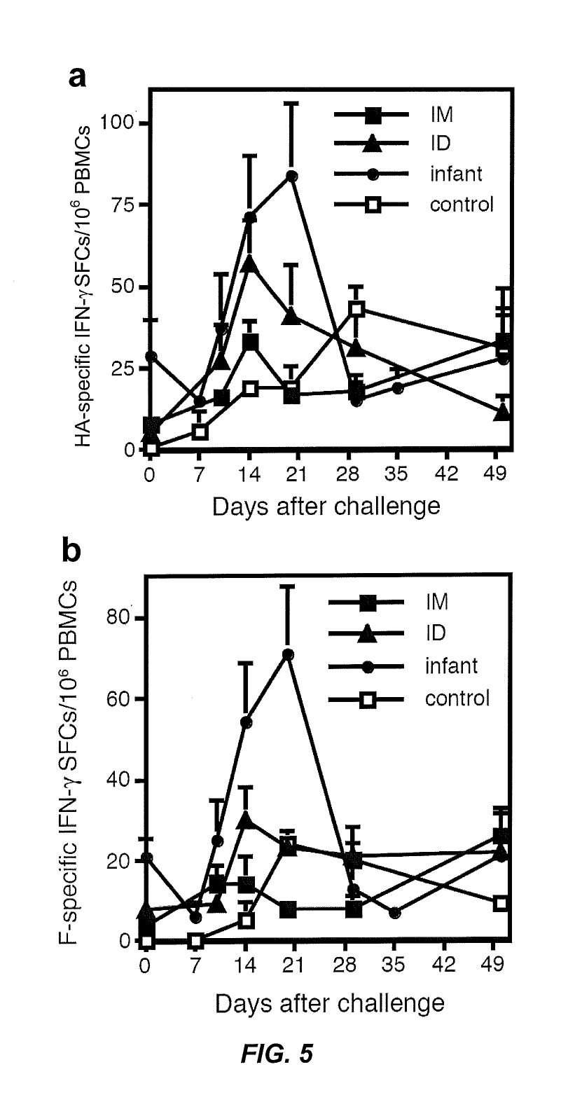

Cellular immune responses after challenge. ELISPOT assays of PBMC production of IFN-γ were used to monitor the HA and F-specific T cell responses to viral challenge. All vaccinated monkeys showed a rapid rise in production of IFN-γ in response to HA or F peptide stimulation that peaked at day 14-20 after challenge, then retracted to a stable level above the pre-challenge baseline. Infant monkeys had the highest IFN-γ production. The development of MV-specific IFN-γ-producing cells was slower for unvaccinated control monkeys with a peak at day 25 indicating an anamnestic response in immunized monkeys (FIGS. 5A and 5B). The peak HA-specific IFN-γ spot number was 33±6 for IM, 57±13 for ID, 84±22 for infant and 43±7 SFC/10⁶ PBMC for control monkeys. The F-specific IFN-γ response was lower than the HA response with mean peak spot numbers of 14±7 for IM, 30±8 for ID, 71±17 for infant, and 24±3 SFC/10⁶ PBMC for control monkeys.

### Discussion

Immunization with Vaxfectin®-formulated, codon-optimized DNAs expressing the MV HA and F proteins elicited strong antibody and T cell responses in mice and rhesus macaques and provided complete protection against rash and viremia after challenge with wild type MV in both juvenile and infant macaques. Two doses of vaccine delivered either intradermally or intramuscularly to juvenile or intradermally to infant rhesus macaques induced MV-specific antibody responses that were durable, neutralizing and of high avidity, as well as MV-specific IFN-γ-producing memory T cells. This is the first DNA-based MV vaccine that has successfully immunized infant macaques and the first to provide complete long-term protection from measles for both infant and juvenile macaques. Therefore, a Vaxfectin®-formulated measles DNA vaccine may be useful as a new measles vaccine for young infants.

In mice, Vaxfectin® formulation of a variety of experimental DNA vaccines improves antibody production up to 100 fold over naked DNA, particularly at low doses, and leads to a more durable response (Hahn, U. K., et al., Vaccine, 24:4595-4597 (2006); Margalith, M., et al., Genet. Vaccines. Ther., 4:2 (2006); Nukuzuma, C., et al., Viral Immunol., 16:183-189 (2003); and Reyes, L., et al., Vaccine, 19:3778-3786 (2001)). In the current example, these advantages were confirmed for DNA expressing MV HA and F. Similar improvements have also been observed in studies of immune responses in rabbits and sheep (Hartikka, J., et al., Vaccine, 19:1911-1923 (2001); Hermanson, G., et al., Proc Natl. Acad Sci U.S.A., 101:13601-13606 (2004); and Sedegah, M., et al., Vaccine, 24:1921-1927 (2006)). Improvement varies with the antigen (Reyes, L., et al., Vaccine, 19:3778-3786 (2001)) and in the current study antibody responses against the MV HA protein were 2-10 times higher for DNA formulated with Vaxfectin® than with PBS. Responses were dose-dependent and had not plateaued at 100 μg of DNA. HA induced 10 times higher IgG titers than F at the same dose and this reflects differences in immunogenicity of the proteins or in the levels of protein expression.

Induction of protective immune responses in nonhuman primates by measles DNA vaccines has been challenging. Previous studies of an unformulated HA+F measles DNA vaccine delivered ID or by gene gun to juvenile macaques showed good T cell responses, antibody responses that were sustained and protection in monkeys with PRNT values >200 mIU/mL at the time of challenge (Polack, F., et al., Nat Med., 6:776-781 (2000)). Formulation of an alphavirus-based DNA vaccine with poly-lactide coglycolide microspheres did not improve immune responses and at low ID doses primed for enhanced disease after challenge (Pan, C. H., et al., Clin Vaccine Immunol, 2008. In Press). Formulation with Vaxfectin® substantially improved the predictability, magnitude and kinetics of the antibody and T cell responses in juvenile macaques. Within one month all juvenile monkeys immunized either ID or IM developed protective levels of neutralizing antibody that were similar to those previously reported in rhesus macaques after immunization with the current live measles vaccine (average of 4943 mIU/mL) (Polack, F., et al., Nat Med., 6:776-781 (2000)). The MV-specific IFN-γ response was also rapid with a peak two weeks after vaccination. A second dose at 4 weeks may be optional based on the showing that the antibody response was still rising at the time of the boost.

Immaturity of the immune system is a barrier to early immunization for measles, as well as other infectious diseases (Bot, A., et al., Microbes. Infect., 4:511-520 (2002)). Previous studies in infant monkeys have shown priming of the immune response by naked DNA, but limited protection from challenge unless boosted with the live virus vaccine (Pasetti, M. F., et al., Clin. Pharmacol. Ther., 82:672-685 (2007); and Stittelaar, K. J., et al., Vaccine, 20:2022-2026 (2002)). Studies of neonatal immunization have been performed in mice. Some studies have suggested that DNA vaccines are tolerizing in neonatal mice (Mor, G., et al., J Clin Invest., 98:2700-2705 (1996)), but most have demonstrated good antibody and T cell responses even in the face of maternal antibody (Bot, A., et al., Microbes. Infect., 4:511-520 (2002); Manickan, E., et al., J Clin Invest., 100:2371-2375 (1997); and Zhang, J., et al., J. Virol., 76:11911-11919 (2002)). In general, the T cell responses in young animals tend to be more skewed toward Th2 cytokines compared to the responses of older animals Manickan, E., et al., J Clin Invest., 100:2371-2375 (1997)). In a previous report, immunization of 1-2 week old macaques with 2 doses of vaccinia virus-vectored MV HA+F induced variable levels of neutralizing antibodies, cytotoxic T cell responses and protection from challenge 12 weeks after boosting (Zhu, Y., et al., Virology, 276:202-213 (2000)). In the current study, antibody responses were similar to juvenile monkeys, but T cell responses were not. Juvenile monkeys developed predominantly IFN-γ-producing T cells with better responses to HA than F, while infant monkeys developed equal numbers of IFN-γ and IL-4-producing T cells and had similar responses to HA and F.

Both infant and juvenile monkeys developed sustained neutralizing antibody titers higher than 120 mIU/mL and were completely protected from measles as determined by development of rash and detection of viremia. However, infant monkeys did develop a transient IgM response and a 10-fold increase in neutralizing antibodies after challenge indicating some virus replication, at least locally in the lung and draining lymph nodes. Although juvenile monkeys showed no increase in IgM, neutralizing antibody or EIA antibodies, there was an increase in the avidity of IgG after challenge, also observed after measles in monkeys previously vaccinated with the current live virus vaccine (Polack, F. P., et al., Nat. Med., 9:1209-1213 (2003)), again indicating the stimulatory effects on immunologic memory of limited virus replication upon re-exposure. Infant monkeys also showed an increase in IFN-γ, but not IL-4 responses after challenge, potentially reflecting maturation of the immune system during the year after vaccination.

The present application provides the first candidate measles DNA vaccine that can elicit rapid and sustained protective responses to measles in infant monkeys as well as juvenile monkeys. A Vaxfectin®-formulated DNA vaccine is a promising approach for development of a new measles vaccine for young children.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All patent documents and references cited herein are incorporated by reference as if fully set forth.

Wild type F Protein (from LOCUS AF266287
RNA linear
Measles virus strain Edmonston (Moraten
vaccine), complete genome
SEQ ID NO: 1
ATGTCCATCATGGGTCTCAAGGTGAACGTCTCTGCCATATTCATGGCAGT
ACTGTTAACTCTCCAAACACCCACCGGTCAAATCCATTGGGGCAATCTCT
CTAAGATAGGGGTGGTAGGAATAGGAAGTGCAAGCTACAAAGTTATGACT
CGTTCCAGCCATCAATCATTAGTCATAAAATTAATGCCCAATATAACTCT
CCTCAATAACTGCACGAGGGTAGAGATTGCAGAATACAGGAGACTACTGA
GAACAGTTTTGGAACCAATTAGAGATGCACTTAATGCAATGACCCAGAAT
ATAAGACCGGTTCAGAGTGTAGCTTCAAGTAGGAGACACAAGAGATTTGC
GGGAGTAGTCCTGGCAGGTGCGGCCCTAGGCGTTGCCACAGCTGCTCAGA
TAACAGCCGGCATTGCACTTCACCAGTCCATGCTGAACTCTCAAGCCATC
GACAATCTGAGAGCGAGCCTGGAAACTACTAATCAGGCAATTGAGACAAT
CAGACAAGCAGGGCAGGAGATGATATTGGCTGTTCAGGGTGTCCAAGACT
ACATCAATAATGAGCTGATACCGTCTATGAACCAACTATCTTGTGATTTA
ATCGGCCAGAAGCTCGGGCTCAAATTGCTCAGATACTATACAGAAATCCT
GTCATTATTTGGCCCCAGTTTACGGGACCCCATATCTGCGGAGATATCTA
TCCAGGCTTTGAGCTATGCGCTTGGAGGAGACATCAATAAGGTGTTAGAA
AAGCTCGGATACAGTGGAGGTGATTTACTGGGCATCTTAGAGAGCGGAGG
AATAAAGGCCCGGATAACTCACGTCGACACAGAGTCCTACTTCATTGTCC
TCAGTATAGCCTATCCGACGCTGTCCGAGATTAAGGGGGTGATTGTCCAC
CGGCTAGAGGGGGTCTCGTACAACATAGGCTCTCAAGAGTGGTATACCAC
TGTGCCCAAGTATGTTGCAACCCAAGGGTACCTTATCTCGAATTTTGATG
AGTCATCGTGTACTTTCATGCCAGAGGGGACTGTGTGCAGCCAAAATGCC
TTGTACCCGATGAGTCCTCTGCTCCAAGAATGCCTCCGGGGGTACACCAA
GTCCTGTGCTCGTACACTCGTATCCGGGTCTTTTGGGAACCGGTTCATTT
TATCACAAGGGAACCTAATAGCCAATTGTGCATCAATCCTTTGCAAGTGT
TACACAACAGGAACGATCATTAATCAAGACCCTGACAAGATCCTAACATA
CATTGCTGCCGATCACTGCCCGGTAGTCGAGGTGAACGGCGTGACCATCC
AAGTCGGGAGCAGGAGGTATCCAGACGCTGTGTACTTGCACAGAATTGAC
CTCGGTCCTCCCATATCATTGGAGAGGTTGGACGTAGGGACAAATCTGGG
GAATGCAATTGCTAAGTTGGAGGATGCCAAGGAATTGTTGGAGTCATCGG
ACCAGATATTGAGGAGTATGAAAGGTTTATCGAGCACTAGCATAGTCTAC
ATCCTGATTGCAGTGTGTCTTGGAGGGTTGATAGGGATCCCCGCTTTAAT
ATGTTGCTGCAGGGGGCGTTGTAACAAAAAGGGAGAACAAGTTGGTATGT
CAAGACCAGGCCTAAAGCCTGATCTTACGGGAACATCAAAATCCTATGTA
AGGTCGCTCTGA
Wild type HA Protein (from LOCUS AF266287
RNA linear
Measles virus strain Edmonston (Moraten
vaccine), complete genome
SEQ ID NO: 2
ATGTCACCACAACGAGACCGGATAAATGCCTTCTACAAAGATAACCCCCA
TCCCAAGGGAAGTAGGATAGTCATTAACAGAGAACATCTTATGATTGATA
GACCTTATGTTTTGCTGGCTGTTCTGTTTGTCATGTTTCTGAGCTTGATC
GGGTTGCTAGCCATTGCAGGCATTAGACTTCATCGGGCAGCCATCTACAC
CGCAGAGATCCATAAAAGCCTCAGCACCAATCTAGATGTAACTAACTCAA
TCGAGCATCAGGTCAAGGACGTGCTGACACCACTCTTCAAAATCATCGGT
GATGAAGTGGGCCTGAGGACACCTCAGAGATTCACTGACCTAGTGAAATT
AATCTCTGACAAGATTAAATTCCTTAATCCGGATAGGGAGTACGACTTCA
GAGATCTCACTTGGTGTATCAACCCGCCAGAGAGAATCAAATTGGATTAT
GATCAATACTGTGCAGATGTGGCTGCTGAAGAGCTCATGAATGCATTGGT
GAACTCAACTCTACTGGAGACCAGAACAACCAATCAGTTCCTAGCTGTCT
CAAAGGGAAACTGCTCAGGGCCCACTACAATCAGAGGTCAATTCTCAAAC
ATGTCGCTGTCCCTGTTAGACTTGTATTTAGGTCGAGGTTACAATGTGTC
ATCTATAGTCACTATGACATCCCAGGGAATGTATGGGGGAACTTACCTAG
TGGAAAAGCCTAATCTGAGCAGCAAAAGGTCAGAGTTGTCACAACTGAGC
ATGTACCGAGTGTTTGAAGTAGGTGTTATCAGAAATCCGGGTTTGGGGGC
TCCGGTGTTCCATATGACAAACTATCTTGAGCAACCAGTCAGTAATGATC
TCAGCAACTGTATGGTGGCTTTGGGGGAGCTCAAACTCGCAGCCCTTTGT
CACGGGGAAGATTCTATCACAATTCCCTATCAGGGATCAGGGAAAGGTGT
CAGCTTCCAGCTCGTCAAGCTAGGTGTCTGGAAATCCCCAACCGACATGC
AATCCTGGGTCCCCTTATCAACGGATGATCCAGTGATAGACAGGCTTTAC
CTCTCATCTCACAGAGGTGTTATCGCTGACAATCAAGCAAAATGGGCTGT
CCCGACAACACGAACAGATGACAAGTTGCGAATGGAGACATGCTTCCAAC
AGGCGTGTAAGGGTAAAATCCAAGCACTCTGCGAGAATCCCGAGTGGGCA
CCATTGAAGGATAACAGGATTCCTTCATACGGGGTCTTGTCTGTTGATCT
GAGTCTGACAGTTGAGCTTAAAATCAAAATTGCTTCGGGATTCGGGCCAT
TGATCACACACGGTTCAGGGATGGACCTATACAAATCCAACCACAACAAT
GTGTATTGGCTGACTATCCCGCCAATGAAGAACCTAGCCTTAGGTGTAAT
CAACACATTGGAGTGGATACCGAGATTCAAGGTTAGTCCCTACCTCTTCA
CTGTCCCAATTAAGGAAGCAGGCGAAGACTGCCATGCCCCAACATACCTA
CCTGCGGAGGTGGATGGTGATGTCAAACTCAGTTCCAATCTGGTGATTCT
ACCTGGTCAAGATCTCCAATATGTTTTGGCAACCTACGATACTTCCAGGG
TTGAACATGCTGTGGTTTATTACGTTTACAGCCCAAGCCGCTCATTTTCT
TACTTTTATCCTTTTAGGTTGCCTATAAAGGGGGTCCCCATCGAATTACA
AGTGGAATGCTTCACATGGGACCAAAAACTCTGGTGCCGTCACTTCTGTG
TGCTTGCGGACTCAGAATCTGGTGGACATATCACTCACTCTGGGATGGTG
GGCATGGGAGTCAGCTGCACAGTCACCCGGGAAGATGGAACCAATCGCAG
ATAG
Codon-optimized Measles F sequence (VR7303)
SEQ ID NO: 3
ATGAGCATCATGGGCCTGAAGGTCAACGTTAGCGCCATCTTCATGGCCGT
GCTGCTGACCCTGCAGACCCCCACCGGCCAGATCCACTGGGGCAACCTGA
GCAAGATCGGCGTGGTGGGCATCGGCAGCGCCAGCTACAAGGTCATGACC
AGAAGTAGCCACCAGAGCCTGGTGATCAAGCTGATGCCCAATATCACCCT
GCTGAACAACTGCACCAGAGTGGAGATCGCCGAGTACAGGAGACTGCTGA
GAACCGTGCTGGAGCCTATTAGGGACGCCCTGAACGCTATGACCCAGAAT
ATCAGACCCGTGCAGAGCGTGGCCAGTAGCAGGAGACACAAGAGATTCGC
CGGCGTGGTGCTGGCCGGCGCCGCCCTGGGCGTGGCCACCGCCGCCCAGA
TCACCGCCGGAATCGCCCTGCACCAGAGTATGCTGAATAGCCAGGCTATC
GACAACCTGAGAGCCAGCCTGGAGACCACCAACCAGGCTATCGAGACCAT
CAGACAGGCCGGCCAGGAGATGATCCTGGCCGTGCAGGGCGTGCAGGACT
ACATCAACAACGAGCTGATCCCTAGCATGAACCAGCTGAGCTGCGACCTG
ATCGGCCAGAAGCTGGGCCTGAAGCTGCTGAGATACTACACCGAGATCCT
GAGCCTGTTCGGCCCCAGCCTGAGAGACCCCATCAGCGCCGAGATTAGCA
TCCAGGCCCTGAGCTACGCCCTGGGCGGCGACATCAACAAGGTCCTGGAG
AAGCTGGGCTACAGCGGCGGCGACCTGCTGGGCATCCTGGAGAGCGGCGG
CATCAAGGCTAGAATCACCCACGTGGACACCGAGAGCTACTTCATCGTGC
TGAGCATCGCCTACCCCACCCTGAGCGAGATCAAGGGCGTGATCGTGCAC
AGACTGGAGGGCGTGAGCTACAACATCGGTAGCCAGGAGTGGTACACCAC
CGTGCCCAAATACGTGGCCACCCAGGGCTACCTGATCAGCAACTTCGACG
AGAGTAGCTGCACCTTCATGCCCGAGGGCACCGTGTGCAGCCAGAACGCC
CTGTACCCCATGAGCCCCCTGCTGCAAGAGTGCCTGAGAGGCTACACCAA
GAGCTGCGCCAGAACCCTGGTCAGCGGCAGCTTCGGCAATAGATTTATCC
TGAGCCAGGGCAACCTGATCGCCAACTGCGCCAGTATCCTGTGCAAGTGC
TACACCACCGGCACCATTATCAACCAGGACCCTGACAAGATCCTGACCTA
TATCGCCGCCGACCACTGCCCCGTGGTGGAGGTGAACGGCGTGACAATCC
AGGTCGGCAGCAGAAGATACCCCGACGCCGTGTACCTGCACAGAATAGAC
CTGGGCCCCCCTATTAGCCTGGAGAGACTGGACGTGGGCACCAACCTGGG
CAACGCTATCGCCAAGCTGGAGGACGCCAAGGAGCTGCTGGAGAGCAGCG
ACCAGATCCTGAGAAGTATGAAGGGCCTGAGTAGCACCAGTATCGTGTAT
ATCCTGATCGCCGTGTGCCTGGGCGGCCTGATCGGAATCCCCGCCCTGAT
CTGCTGCTGCCGGGGCAGATGCAACAAGAAGGGCGAGCAGGTCGGAATGA
GCAGACCCGGCCTGAAGCCTGACCTGACCGGCACCAGCAAGAGCTACGTC
AGAAGCCTGTGA
Codon-Optimized Measles HA sequence (VR7302)
SEQ ID NO: 4
ATGAGCCCCCAGAGAGACAGAATCAACGCCTTCTACAAGGATAACCCCCA
CCCCAAGGGCAGCAGAATCGTGATCAACAGAGAGCACCTGATGATCGACA
GACCCTACGTGCTGCTGGCCGTGCTGTTCGTGATGTTCCTGAGCCTGATC
GGCCTGCTGGCCATCGCCGGCATTAGACTGCACAGAGCCGCCATCTACAC
CGCCGAGATCCACAAGAGCCTGAGCACCAACCTGGACGTGACCAACAGCA
TCGAGCACCAGGTCAAGGACGTCCTGACCCCCCTGTTCAAGATCATCGGT
GACGAGGTGGGCCTGAGAACCCCCCAGAGATTCACCGACCTGGTGAAGCT
GATCAGCGACAAGATCAAGTTCCTGAACCCCGACAGAGAGTACGACTTCA
GAGACCTGACCTGGTGTATCAACCCCCCCGAGAGAATCAAGCTGGACTAT
GACCAGTACTGCGCCGACGTGGCCGCCGAGGAGCTGATGAACGCCCTGGT
GAACAGCACCCTGCTGGAGACCAGAACCACCAACCAGTTCCTGGCCGTGA
GCAAGGGCAACTGCAGCGGCCCCACCACCATCAGAGGCCAGTTTAGCAAT
ATGAGCCTGAGCCTGCTGGACCTGTACCTGGGCAGAGGCTACAACGTCAG
CAGCATCGTGACCATGACCAGCCAGGGCATGTACGGCGGCACCTACCTGG
TGGAGAAGCCCAACCTGAGTAGCAAGAGAAGCGAGCTGAGCCAGCTGAGC
ATGTACAGAGTGTTCGAGGTCGGCGTGATCAGAAACCCCGGCCTGGGCGC
CCCCGTGTTCCACATGACCAACTACCTGGAGCAGCCCGTGAGCAATGACC
TGAGCAACTGCATGGTGGCCCTGGGCGAGCTGAAGCTGGCCGCCCTGTGC
CACGGCGAGGACAGCATCACCATCCCCTACCAAGGCAGCGGCAAGGGCGT
GAGCTTCCAGCTGGTGAAGCTGGGCGTGTGGAAGAGCCCCACTGACATGC
AGAGCTGGGTGCCCCTGAGCACCGACGACCCCGTGATCGACAGACTGTAC
CTGAGCAGCCACAGAGGCGTGATCGCCGACAACCAGGCCAAGTGGGCCGT
GCCCACCACTAGAACCGACGACAAGCTGAGAATGGAGACCTGCTTCCAGC
AGGCCTGCAAGGGCAAGATCCAGGCCCTGTGCGAGAACCCCGAGTGGGCC
CCCCTGAAGGACAACAGAATCCCTAGCTACGGCGTGCTGAGCGTGGACCT
GAGCCTGACCGTGGAGCTGAAGATCAAGATCGCCAGCGGCTTCGGCCCCC
TGATCACCCACGGTAGCGGCATGGACCTGTACAAGAGCAACCACAACAAC
GTGTACTGGCTGACCATCCCCCCCATGAAGAACCTGGCCCTGGGCGTGAT
CAACACCCTGGAGTGGATTCCCAGATTCAAAGTTAGCCCCTACCTGTTCA
CCGTGCCCATCAAGGAGGCCGGCGAGGACTGCCACGCCCCCACCTACCTG
CCCGCCGAGGTGGACGGCGACGTGAAGCTGAGTAGCAACCTGGTGATCCT
GCCCGGCCAGGACCTGCAGTATGTTCTGGCCACCTACGACACCAGCAGAG
TGGAGCACGCCGTGGTGTACTACGTGTATAGCCCCAGCAGAAGCTTCAGC
TACTTCTACCCCTTCCGGCTGCCCATAAAGGGCGTGCCCATCGAGCTGCA
GGTGGAGTGCTTCACCTGGGACCAGAAGCTGTGGTGTAGACACTTCTGCG
TGCTGGCCGACAGCGAGAGCGGCGGCCACATCACCCACAGCGGCATGGTG
GGCATGGGCGTGAGCTGCACCGTGACCAGAGAGGACGGCACCAACAGAAG
ATGA

What is claimed is:

1. A method for immunizing an infant mammal against a target measles virus antigen, comprising inoculating the mammal, while an infant, with an effective amount of a recombinant nucleic acid molecule encoding a peptide comprising one or more relevant epitopes of the target antigen in a cationic lipid adjuvant and pharmaceutical carrier, such that a therapeutically effective amount of the relevant peptide is expressed in the infant mammal, wherein said infant is immunized.

2. The method of claim 1, wherein maternal antibodies are present in detectable amounts in the infant mammal.

3. The method of claim 1, wherein the mammal is a human having an age extending from birth to the age of twelve months.

4. The method of claim 1, wherein the mammal is a human having an age extending from birth to the age of one month.

5. The method of claim 1, wherein the infant mammal is a neonate.

6. The method of claim 1, wherein said target antigen is selected from Hemagglutinin (HA) protein.

7. A method for immunizing an infant mammal against a target measles virus antigen, comprising inoculating the mammal with a therapeutically effective amount of a recombinant nucleic acid molecule encoding a peptide comprising one or more relevant viral epitopes of the target antigen in a cationic lipid adjuvant and a pharmaceutical acceptable carrier, wherein; (i) the therapeutical effective amount of nucleic acid is introduced by a plurality of inoculations all administered while the mammal is an infant; and (ii) immunization results in an enhanced immunity to measles virus infection.

8. The method of claim 7, wherein the mammal is a human.

9. The method of claim 7, wherein the mammal is a human and the first of the plurality of injections is administered at an age extending from birth to about six months.

10. The method of claim 7, wherein the mammal is a human and the first of the plurality of injections is administered at an age extending from birth to about one month.

11. The method of claim 7, wherein the mammal is a human and the first of the plurality of injections is administered at an age extending from birth to about one week.

12. The method of claim 7, wherein the target measles virus antigen is HA protein.

13. The method of claim 1, wherein the adjuvant comprises (±)-N-(3-aminopropyl)-N,N-dimethyl-2,3-bis(syn-9-tetradeceneyloxy)-1-propanaminium bromide (GAP-DMORIE) cationic lipid; and wherein said adjuvant further comprises one or more co-lipids selected from the group consisting of: a neutral lipid; a cytokine; mono-phosphoryl lipid A and trehalosedicorynomycolate AF (MPL+TDM); a solubilized mono-phosphoryl lipid A formulation; and 1,2-diphytanoyl-sn-glycero-3-phosphoethanolamine (DPyPE).

14. The method of claim 13 wherein the adjuvant comprises a GAP-DMORIE cationic lipid and a (DPyPE) co-lipid.


SEQUENCE LISTING (4 sequences)

SEQ ID NO: 1 - DNA, 1662 bp, Measles virus
atgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctga

SEQ ID NO: 2 - DNA, 1854 bp, Measles virus
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatag

SEQ ID NO: 3 - DNA, 1662 bp, Measles virus
atgagcatcatgggcctgaaggtcaacgttagcgccatcttcatggccgtgctgctgaccctgcagacccccaccggccagatccactggggcaacctgagcaagatcggcgtggtgggcatcggcagcgccagctacaaggtcatgaccagaagtagccaccagagcctggtgatcaagctgatgcccaatatcaccctgctgaacaactgcaccagagtggagatcgccgagtacaggagactgctgagaaccgtgctggagcctattagggacgccctgaacgctatgacccagaatatcagacccgtgcagagcgtggccagtagcaggagacacaagagattcgccggcgtggtgctggccggcgccgccctgggcgtggccaccgccgcccagatcaccgccggaatcgccctgcaccagagtatgctgaatagccaggctatcgacaacctgagagccagcctggagaccaccaaccaggctatcgagaccatcagacaggccggccaggagatgatcctggccgtgcagggcgtgcaggactacatcaacaacgagctgatccctagcatgaaccagctgagctgcgacctgatcggccagaagctgggcctgaagctgctgagatactacaccgagatcctgagcctgttcggccccagcctgagagaccccatcagcgccgagattagcatccaggccctgagctacgccctgggcggcgacatcaacaaggtcctggagaagctgggctacagcggcggcgacctgctgggcatcctggagagcggcggcatcaaggctagaatcacccacgtggacaccgagagctacttcatcgtgctgagcatcgcctaccccaccctgagcgagatcaagggcgtgatcgtgcacagactggagggcgtgagctacaacatcggtagccaggagtggtacaccaccgtgcccaaatacgtggccacccagggctacctgatcagcaacttcgacgagagtagctgcaccttcatgcccgagggcaccgtgtgcagccagaacgccctgtaccccatgagccccctgctgcaagagtgcctgagaggctacaccaagagctgcgccagaaccctggtcagcggcagcttcggcaatagatttatcctgagccagggcaacctgatcgccaactgcgccagtatcctgtgcaagtgctacaccaccggcaccattatcaaccaggaccctgacaagatcctgacctatatcgccgccgaccactgccccgtggtggaggtgaacggcgtgacaatccaggtcggcagcagaagataccccgacgccgtgtacctgcacagaatagacctgggcccccctattagcctggagagactggacgtgggcaccaacctgggcaacgctatcgccaagctggaggacgccaaggagctgctggagagcagcgaccagatcctgagaagtatgaagggcctgagtagcaccagtatcgtgtatatcctgatcgccgtgtgcctgggcggcctgatcggaatccccgccctgatctgctgctgccggggcagatgcaacaagaagggcgagcaggtcggaatgagcagacccggcctgaagcctgacctgaccggcaccagcaagagctacgtcagaagcctgtga

SEQ ID NO: 4 - DNA, 1854 bp, Measles virus
atgagcccccagagagacagaatcaacgccttctacaaggataacccccaccccaagggcagcagaatcgtgatcaacagagagcacctgatgatcgacagaccctacgtgctgctggccgtgctgttcgtgatgttcctgagcctgatcggcctgctggccatcgccggcattagactgcacagagccgccatctacaccgccgagatccacaagagcctgagcaccaacctggacgtgaccaacagcatcgagcaccaggtcaaggacgtcctgacccccctgttcaagatcatcggtgacgaggtgggcctgagaaccccccagagattcaccgacctggtgaagctgatcagcgacaagatcaagttcctgaaccccgacagagagtacgacttcagagacctgacctggtgtatcaacccccccgagagaatcaagctggactatgaccagtactgcgccgacgtggccgccgaggagctgatgaacgccctggtgaacagcaccctgctggagaccagaaccaccaaccagttcctggccgtgagcaagggcaactgcagcggccccaccaccatcagaggccagtttagcaatatgagcctgagcctgctggacctgtacctgggcagaggctacaacgtcagcagcatcgtgaccatgaccagccagggcatgtacggcggcacctacctggtggagaagcccaacctgagtagcaagagaagcgagctgagccagctgagcatgtacagagtgttcgaggtcggcgtgatcagaaaccccggcctgggcgcccccgtgttccacatgaccaactacctggagcagcccgtgagcaatgacctgagcaactgcatggtggccctgggcgagctgaagctggccgccctgtgccacggcgaggacagcatcaccatcccctaccaaggcagcggcaagggcgtgagcttccagctggtgaagctgggcgtgtggaagagccccactgacatgcagagctgggtgcccctgagcaccgacgaccccgtgatcgacagactgtacctgagcagccacagaggcgtgatcgccgacaaccaggccaagtgggccgtgcccaccactagaaccgacgacaagctgagaatggagacctgcttccagcaggcctgcaagggcaagatccaggccctgtgcgagaaccccgagtgggcccccctgaaggacaacagaatccctagctacggcgtgctgagcgtggacctgagcctgaccgtggagctgaagatcaagatcgccagcggcttcggccccctgatcacccacggtagcggcatggacctgtacaagagcaaccacaacaacgtgtactggctgaccatcccccccatgaagaacctggccctgggcgtgatcaacaccctggagtggattcccagattcaaagttagcccctacctgttcaccgtgcccatcaaggaggccggcgaggactgccacgcccccacctacctgcccgccgaggtggacggcgacgtgaagctgagtagcaacctggtgatcctgcccggccaggacctgcagtatgttctggccacctacgacaccagcagagtggagcacgccgtggtgtactacgtgtatagccccagcagaagcttcagctacttctaccccttccggctgcccataaagggcgtgcccatcgagctgcaggtggagtgcttcacctgggaccagaagctgtggtgtagacacttctgcgtgctggccgacagcgagagcggcggccacatcacccacagcggcatggtgggcatgggcgtgagctgcaccgtgaccagagaggacggcaccaacagaagatga